SEQUENCE LISTING (73 sequences)

SEQ ID NO: 1 - protein, 654 aa, Homo sapiens
MYSSSCETTRNTTGIEESTDGMILGPEDLSYQIYDVSGESNSAVSTEDLKECLKKQLEFCFSRENLSKDLYLISQMDSDQFIPIWTVANMEEIKKLTTDPDLILEVLRSSPMVQVDEKGEKVRPSHKRCIVILREIPETTPIEEVKGLFKSENCPKVISCEFAHNSNWYITFQSDTDAQQAFKYLREEVKTFQGKPIMARIKAINTFFAKNGYRLMDSSIYSHPIQTQAQYASPVFMQPVYNPHQQYSVYSIVPQSWSPNPTPYFETPLAPFPNGSFVNGFNSPGSYKTNAAAMNMGRPFQKNRVKPQFRSSGGSEHSTEGSVSLGDGQLNRYSSRNFPAERHNPTVTGHQEQTYLQKETSTLQVEQNGDYGRGRRTLFRGRRRREDDRISRPHPSTAESKAPTPKFDLLASNFPPLPGSSSRMPGELVLENRMSDVVKGVYKEKDNEELTISCPVPADEQTECTSAQQLNMSTSSPCAAELTALSTTQQEKDLIEDSSVQKDGLNQTTIPVSPPSTTKPSRASTASPCNNNINAATAVALQEPRKLSYAEVCQKPPKEPSSVLVQPLRELRSNVVSPTKNEDNGAPENSVEKPHEKPEARASKDYSGFRGNIIPRGAAGKIREQRRQFSHRAIPQGVTRRNGKEQYVPPRSPK

SEQ ID NO: 2 - protein, 72 aa, Homo sapiens
MGVELMMELEPLQGNEETRALFMPREDTARPQSASQEESSREPDHAGTLIVDISTSRTVIQNAYVSLEETLK

SEQ ID NO: 3 - protein, 367 aa, Homo sapiens
MLPPRRLQTLLRQAVELQRDRCLYHNTKLDNNLDSVSLLIDHVCSRRQFPCYTQQILTEHCNEVWFCKFSNDGTKLATGSKDTTVIIWQVDPDTHLLKLLKTLEGHAYGVSYIAWSPDDNYLVACGPDDCSELWLWNVQTGELRTKMSQSHEDSLTSVAWNPDGKRFVTGGQRGQFYQCDLDGNLLDSWEGVRVQCLWCLSDGKTVLASDTHQRIRGYNFEDLTDRNIVQEDHPIMSFTISKNGRLALLNVATQGVHLWDLQDRVLVRKYQGVTQGFYTIHSCFGGHNEDFIASGSEDHKVYIWHKRSELPIAELTGHTRTVNCVSWNPQIPSMMASASDDGTVRIWGPAPFIDHQNIEEECSSMDS

SEQ ID NO: 4 - protein, 188 aa, Homo sapiens
MDVNIAPLRAWDDFFPGSDRFARPDFRDISKWNNRVVSNLLYYQTNYLVVAAMMISIVGFLSPFNMILGGIVVVLVFTGFVWAAHNKDVLRRMKKRYPTTFVMVVMLASYFLISMFGGVMVFVFGITFPLLLMFIHASLRLRNLKNKLENKMEGIGLKRTPMGIVLDALEQQEEGINRLTDYISKVKE

SEQ ID NO: 5 - protein, 398 aa, Homo sapiens
MMLKGITRLISRIHKLDPGRFLHMGTQARQSIAAHLDNQVPVESPRAISRTNENDPAKHGDQHEGQHYNISPQDLETVFPHGLPPRFVMQVKTFSEACLMVRKPALELLHYLKNTSFAYPAIRYLLYGEKGTGKTLSLCHVIHFCAKQDWLILHIPDAHLWVKNCRDLLQSSYNKQRFDQPLEASTWLKNFKTTNERFLNQIKVQEKYVWNKRESTEKGSPLGEVVEQGITRVRNATDAVGIVLKELKRQSSLGMFHLLVAVDGINALWGRTTLKREDKSPIAPEELALVHNLRKMMKNDWHGGAIVSALSQTGSLFKPRKAYLPQELLGKEGFDALDPFIPILVSNYNPKEFESCIQYYLENNWLQHEKAPTEEGKKELLFLSNANPSLLERHCAYL

SEQ ID NO: 6 - protein, 261 aa, Homo sapiens
MAGPELLLDSNIRLWVVLPIVIITFFVGMIRHYVSILLQSDKKLTQEQVSDSQVLIRSRVLRENGKYIPKQSFLTRKYYFNNPEDGFFKKTKRKVVPPSPMTDPTMLTDMMKGNVTNVLPMILIGGWINMTFSGFVTTKVPFPLTLRFKPMLQQGIELLTLDASWVSSASWYFLNVFGLRSIYSLILGQDNAADQSRMMQEQMTGAAMAMPADTNKAFKTEWEALELTDHQWALDDVEEELMAKDLHFEGMFKKELQTSIF

SEQ ID NO: 7 - protein, 403 aa, Homo sapiens
MSGRVGDLSPRQKEALAKFRENVQDVLPALPNPDDYFLLRWLRARSFDLQKSEAMLRKHVEFRKQKDIDNIISWQPPEVIQQYLSGGMCGYDLDGCPVWYDIIGPLDAKGLLFSASKQDLLRTKMRECELLLQECAHQTTKLGRKVETITIIYDCEGLGLKHLWKPAVEAYGEFLCMFEENYPETLKRLFVVKAPKLFPVAYNLIKPFLSEDTRKKIMVLGANWKEVLLKHISPDQVPVEYGGTMTDPDGNPKCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

SEQ ID NO: 8 - protein, 251 aa, Homo sapiens
MDMKRRIHLELRNRTPAAVRELVLDNCKSNDGKIEGLTAEFVNLEFLSLINVGLISVSNLPKLPKLKKLELSENRIFGGLDMLAEKLPNLTHLNLSGNKLKDISTLEPLKKLECLKSLDLFNCEVTNLNDYRESVFKLLPQLTYLDGYDREDQEAPDSDAEVDGVDEEEEDEEGEDEEDEDDEDGEEEEFDEEDDEDEDVEGDEDDDEVSEEEEEFGLDEEDEDEDEDEEEEEGGKGEKRKRETDDEGEDD

SEQ ID NO: 9 - protein, 151 aa, Homo sapiens
MPRGSRSRTSRMAPPASRAPQMRAAPRPAPVAQPPAAAPPSAVGSSAAAPRQPGLMAQMATTAAGVAVGSAVGHTLGHAITGGFSGGSNAEPARPDITYQEPQGTQPAQQQQPCLYEIKQFLECAQNQGDIKLCEGFNEVLKQCRLANGLA

SEQ ID NO: 10 - DNA, 6497 bp, Homo sapiens
ccgggtggaggggcaaggcgagtgtgtgtccttatcctagcaattggggcgcgggcctgtgagccagttggagttgcggcggcgggaacgattgggctgagcagaggacgacatgttgcttttcgtggagcatttatggggttaagtggcatgggatttctgtttctgatagtaaatagcaggtagcatctaaaggaactggtttaaatcctaatgccaaagtatggcaagaaattgctcctggaaatactgatgccaccccagtaactcatggaactgaaagctcttggcatgaaatagcagctacatcaggtgctcatcctgagggtaatgcagagctctcagaagatatatgtaaagaatatgaagtaatgtattcttcatcttgtgaaaccacaagaaatactacaggcattgaagaatcaactgatgggatgattttaggaccagaagatctgagttaccaaatatatgatgtttccggagaaagcaattcagcagtttctacagaagacctaaaagaatgtctgaagaaacaattagaattctgtttttcacgagaaaatttgtcaaaggatctttacttgatatctcaaatggatagtgatcagttcatcccaatttggacagttgccaacatggaagaaataaaaaagttgactacagaccctgatctaattcttgaagtgttaagatcttctcccatggtacaagttgatgagaagggtgagaaagtgagaccaagtcataagcgttgtattgtaattcttagagagattcctgaaacaacaccaatagaggaagtgaaaggtttgttcaaaagtgaaaactgccccaaagtgataagctgtgagtttgcacacaatagcaactggtatatcactttccagtcagacacagatgcacaacaggcttttaaatacttaagagaagaagttaaaacatttcagggcaagccaattatggcaaggataaaagccatcaatacattttttgctaagaatggttatcgattaatggattctagtatctatagtcaccccattcaaactcaagcacagtatgcctccccagtctttatgcagcctgtatataatcctcaccaacagtactcggtctatagtattgtgcctcagtcttggtctccaaatcctacaccttactttgaaacaccactggctccctttcccaatggtagttttgtgaatggctttaattcgccaggatcttataaaacaaatgctgctgctatgaatatgggtcgaccattccaaaaaaatcgtgtgaagcctcagtttaggtcatctggtggttcagaacactcaacagagggctctgtatccttgggggatggacagttgaacagatatagttcaagaaactttccagctgaacggcataaccccacagtaactgggcatcaggagcaaacttaccttcagaaggagacttccactttgcaggtggaacagaatggggactatggtaggggcaggagaactctcttcagaggtcgaagacgacgagaagatgacaggatctcaagacctcatccttcaacagctgaatcaaaggctccaacaccaaagtttgacttattagcctcaaattttccacctttacctggaagttcatcaagaatgccaggtgaactcgttttggagaataggatgtctgatgttgttaaaggtgtctacaaagaaaaggataatgaagagttgacaattagttgcccagtgcctgcagatgagcagacagaatgcacttctgcccagcaactcaatatgagtaccagttctccatgtgctgctgagcttactgcattaagcacaactcagcaagaaaaggatctaatagaagattcctctgttcagaaggatggtctcaatcagacaactataccagtttctcctccaagtactacaaagccatcgagggcaagtactgcttcaccatgtaataataacataaatgcagctacagctgtggctctacaggaaccccgaaagttaagttatgctgaagtgtgccagaagccccctaaagagccatcttcagttcttgtgcagccactacgggaacttcgctccaatgtggtgtctcccaccaaaaatgaagacaatggagctcctgagaactccgttgagaaaccacatgagaagccagaagcaagggctagtaaggattattctggcttccgaggcaatataatccccaggggagcagcaggaaaaatcagggaacagagacgccagtttagccatagggctatacctcagggagtgactcgacgtaatggcaaagagcaatatgtgccacccagatcaccaaagtaaaaaacaacaaaactattcaaaaacttcactctcttcccattaaacttgaactgtggctatattgaactgttttggaggggagggggtagccaggaaggaaacaagagaaagtacgtccatttcattatggattttggagttgtgagtgataggatcccaaaattcatctctaatgtggtttttaaatgctggaggattccaatcaatataaatatatatatatatatacacacacatatataaaaagtataatttttctatttttgtttttggttttaatttgcagagatttgctgccaggaatcaattttgagggttcagatttagcttggaagaaaaaaaagaaacatacatccttcagtataggagatgagggaatgagagaaaatattttttgaagaagcatttctgtaaaattagaaattactttttttaatctatttaaagtttggcttgaagaatgccatctctgactatatggccttgtattgcaaagcagatcagtggctggggtgcctgttgtgggtgtgagtgtgtacaagagcgattgaagccaaatctgttgtcatgttagtaaatgatttgaaaactgaatgtaatacttgagtagatttttttttctagtttgaaatttagtctgtctttttgaccttactaatatttcatttaacaagttgtaaaactctgattgtacttagagatgtgactaccaatcagtttgatactcaaggaaagggggttattcaagaaattgaaaatttcatcttggacctcagtgcatcggtcaaatggatttcagaggtttaaacttccctgtgattccccctgaatacccccaaaatgagaaacaaaattttttttcttactccatttgttactctctgttctttgactgcccacccacagaaaagcaaaataaccaactacctactcaattgtgtgtttgtaattgctttgagcagtctagtcaaatcatataaattgttctaaatttcagaattgaacattgaagtattaactcttctgttcacacatttagaattttagctcccaagatggtagggcagactgaccgtacagtaatttatttgtcgttagtgttaaagattaagcatagtaactgactcttaagtgttaaataatgtagaagtaaaaaaattttttttaaaggcttaatttgggaggggggacttatttctgtttacagtgtattaccttccttccctcctcttctccccccacacccaacaaaatacagtttggaattcactgaaacagtaccagcaagtcatgagattttttagtaaagatgagaaagatggttgaagaaaattagtgcataatttctcagtgaataaagttgtagctctcatatactaaatagacaagtttacatgctgttatttagaaaatgactaaaatattaaaaaccgtgttgtgttaatctgttttaagtcataccatgttcagagttctatgtaaggtgggttttatttttcttttaagggatagtttgtaatagtaagaactgtcccatatgttagtaaattacatatgtacaaattgaaactgtaaattgtgaacactggaaagcaccattgtgacatagagtaaacatcttagtaatatattaaagtgaatgtaaatggtggttaaaattacattactgtgaaattcatcttccaactctaagttaagctttggagatacatgttagtggttaactgttaagagctttgaaaacactgcacatatctgtacaagccagaattactatttctttgacttattattagcttggcagttgcttttgatttgattgttttatgacatggtatactactatatttactcagtttgaaactattcatttctacacactatttttaaaaattgcctactaggtgaaacataacaataaaactacctgtgctgaaatttgggggaagtttaggtcctttaaaaaaacatattaatcattgactacatctatgataaaagtgcttattttggtttactaagataatgcagttggtggaaatgataaacgttttaagtgttaacatcctttgaatgcgttggatttcagagaataaacattttgtaaaaatcacttggtaaggattataaacttaattactgcacttaaaatgaaacattactttttttaaacaatgtgtcacaaatgtaggtctgtattacttgtatgcttgtgtgacttactgttagtccagctctaaaaatttaaaggttgtaattgaaatacaagaaaagagccttcttttagaagaaagcaagtatatttttgcttttacttcaaatgttatttaaagtagaaatttaatttgtagatataacctttaaaaattttctcattaagacaatgtttttaatttaatttgcctcattacatctaatagttcccatttgatggcatgtatagggaagagtgagagagtgtgtgtgtgtgtatgtgtgtgtaatatttatatatattcacagtatgtatttagcatttattttattacagcagatttaaagtttgtatctaaataatgcctatgagttgtgtgaagctcttggctttttttccaacgttactttgtaactaatgagggtggatgttcattgtagtttatttatttggttctttagatggaggaatttaaaaaatcaaatttttctcttcacctttatgacttgacatttccttgatctgttggaggctaaaagtaggtataaatgatattgaatgttgggtatagtgatactctgccatagttcttactgcatgaagagaacaagagtcacacaagttcaccactttgcacttcatagagaaggtacatagagacattgcaaaacctgtctccatttgctatcctgataattaaggttttcataatacctagggcctgtctctgagtaattttaattttgccaaatacactgacatttaaaatagtgatccatctaaatttttttcagctgggttttgaggaatataagagctttcaatgataaaggtttgttgtagttgtcttatgtgctgaatttgcagatgatcagatgctgtgcagaattctgatttatttttgtttcctaaaattaagatagcttgaatattatttcacattcctttttcttttttaaataaacaggtttgctttggaaaggcttaatgatggaatgttagcatcttcactagggtaaagaagaacaaaaagaatgttgctggaacgtaaaatagtatttaaaagttaatgaacacttctctagttttcttagttatggccttaataattagtctcttggcttaaatgtccactggttttactttgacacagttgaacaacactggggttaagtctctggtatttaggctggcaatatatatattaaccatattttaaaagtaccaattttgtttttacagaaaagataaaactcaaaagagaacagtgtattccttctgaggggcttttataaattattaactataatatatgatggattttttcctaattttttatatttccttacaattttggtggccattaatttaactttaggcttttgggcatatgctagtctgagcttccgaaaagatacatatatgtttcccttttcattagctgaatgaggatattttaagaagttgaaagagaatttattttcaagttgtgagtaaatcctcctttgaaattcacctgattattagataacttaaagtttatttttaaaagctgacaactttttatgaatcttcgagttgacagttcctaaaagcgtaactcagatattaatgggctgtgtattaaatggttttattttcagttttgcagcacagaacactgttgaaatatccatatcaacttgatttttttaacctaattcaggtgtcctttgcatctcttaaatgttgggggtgggggtcagagccagttatccggcttctgttttgtcgattgcttagatttgttcctgttgtcaaaactgttacccccaaaattggtgtgacacatgctcatgcataaaatgttaaaatgagtacatccttgtatttgtatttgttttcaacatcgccaaggtgctatgggaaattaacaaaattagaaaaaaaataaaattattaaaaagcaaaaaaaaaaaaaaaaaaaa

SEQ ID NO: 11 - DNA, 484 bp, Homo sapiens
atgggggtggaactcatgatggaattggagcctttacaagggaatgaagagacaagagctctctttatgccacgtgaggatacagcaaggccccaatctgcaagccaggaagagtcgtcacgagaaccagaccatgcaggaactctgatcgtggacatttcaacctccagaactgtgatccaaaatgcatatgtatctttggaagaaactctgaagtaaaggccggaatattctttgtttaaaacattaaaaacaaaacagaccaaagcatcaagcaagaagtttcctggcaataaactaagcacagcattattttttaaggaacacaaattaagtgttcaacctgtggcaaatttgtactttctccctgaattatgttgttatcaaagaaaaaaattgggaagcatggcaaaatatcatcaaaactgaaactagaattaaactaaattaaaataaaaaaaaaaaaaaaaaaaaaaaaaaaaaa

SEQ ID NO: 12 - DNA, 1904 bp, Homo sapiens
ctacgtgcaaaagcagaatgggaaggctaagggacagcttcccgatctaaactattggataaacttcagacctatttaccaccatcagtgatgcttcccccacggcgtttacagactctcctgcggcaggcggtggaactacaaagggatcggtgcctatatcacaataccaaacttgataataatctagattctgtgtctctgcttatagaccatgtttgtagtaggaggcagttcccatgttatacgcagcagatacttacggagcattgtaatgaagtgtggttctgtaaattctctaatgatggcactaaactagcaacaggatcaaaagatacaacagttatcatatggcaagttgatccggatacacacctgctaaaactgcttaaaacattagaaggacatgcttatggcgtttcttatattgcatggagtccagatgacaactatcttgttgcttgtggcccagatgactgctctgagctttggctttggaatgtacaaacaggagaactaaggacaaaaatgagccagtctcatgaagacagtttgacaagtgtggcttggaatccagatgggaagcgctttgtgactggaggtcagcgtgggcagttctatcagtgtgacttagatggtaatctccttgactcctgggaaggggtaagagtgcaatgcctttggtgcttgagtgatggaaagactgttctggcatcagatacacaccagcgaattcggggctataacttcgaggaccttacagataggaacatagtacaagaagatcatcctattatgtcttttactatttcaaaaaatggccgattagctttgttaaatgtagcaactcagggagttcatttatgggacttgcaagacagagttttagtaagaaagtatcaaggtgttacacaagggttttatacaattcattcatgttttggaggccataatgaagacttcatcgctagtggcagtgaagatcacaaggtttacatctggcacaaacgtagtgaactgccaattgcggagctgacagggcacacacgtacagtaaactgtgtgagctggaacccacagattccatccatgatggccagcgcctcagatgatggcactgttagaatatggggaccagcaccttttatagaccaccagaatattgaagaggaatgcagtagcatggatagttgatggtgaatttggagcagacgacctctgtttaacttaaaattagtcgtattttaatggcttgggatttggtgcaaacaaacatgattgatagctggacagacatgctcgtcatgaaaaaagaaccatttctgaagcccgattggggccaaacatttacaccttgcttcatagtaaccagttgagatgaagcacgtcgttagaacgttgttggacaccatgttgaattattcccccatcggttgtgaagaactgtgctacattcaggcttacccattgaactcagtatatatatttttttccttcctgtcttttgtctggcaggataccattcttgttgctcttctgtgtaatgaagtttaaatgcttgtttggaaaactttatttaacagtttagaaggcttgatagaaagagtgcattagtctgaagagtatacattggataggaaagaatttccttcttttgtttctccaaatctttccgccttatttagcttgagatctttgcagcttggttcatggattctagccttgcccgttgcgcagtatatactgatccagatgataaaccagtgaactatgtcaaaagcactctcaatattacatttgacaaaaagttttgtacttttcacatagcttgttgccccgtaaaagggttaacagcacaattttttaaaaataaattaagaagtatttaaaaaaaaaaaaaaaaa

SEQ ID NO: 13 - DNA, 2088 bp, Homo sapiens
cgctgtcaactctccaactcagctcagctgatcggttgccgccgccgccgccgccagattctggaggcgaagaacgcaaagctgagaacatggacgttaatatcgccccactccgcgcctgggacgatttcttcccgggttccgatcgctttgcccggccggacttcagggacatttccaaatggaacaaccgcgtagtgagcaacctgctctattaccagaccaactacctggtggtggctgccatgatgatttccattgtggggtttctgagtcccttcaacatgatcctgggaggaatcgtggtggtgctggtgttcacagggtttgtgtgggcagcccacaataaagacgtccttcgccggatgaagaagcgctaccccacgacgttcgttatggtggtcatgttggcgagctatttccttatctccatgtttggaggagtcatggtctttgtgtttggcattacttttcctttgctgttgatgtttatccatgcatcgttgagacttcggaacctcaagaacaaactggagaataaaatggaaggaataggtttgaagaggacaccgatgggcattgtcctggatgccctagaacagcaggaagaaggcatcaacagactcactgactatatcagcaaagtgaaggaataaacataacttacctgagctagggttgcagcagaaattgagttgcagcttgcccttgtccagacctatgttctgcttgcgtttttgaaacaggaggtgcacgtaccacccaattatctatggcagcatgcatgtataggccgaactattatcagctctgatgtttcagagagaagacctcagaaaccgaaagaaaaccaccaccctcctattgtgtctgaagtttcacgtgtgtttatgaaatctaatgggaaatggatcacacgatttctttaagggaattaaaaaaaataaaagaattacggcttttacagcaacaatacgattatcttataggaaaaaaaaaaatcattgtaaagtatcaagacaatacgagtaaatgaaaaggctgttaaagtagatgacatcatgtgttagcctgttcctaaatccctagaattgtaatgtgtgggatataaattagtttttattattctcttaaaaatcaaagatgatctctatcactttgccacctgtttgatgtgcagtggaaactggttaagccagttgttcatacttcctttacaaatataaagatagctgtttaggatattttgttacatttttgtaaatttttgaaatgctagtaatgtgttttcaccagcaagtatttgttgcaaacttaatgtcattttccttaagatggttacagctatgtaacctgtattattctggacggacttattaaaatacaaacagacaaaaaataaaacaaaacttgagttctatttaccttgcacattttttgttgttacagtgaaaaaaatggtccaagaaaatgtttgccatttttgcattgtttcgtttttaactggaacatttagaaagaaggaaatgaatgtgcattttattaattccttaggggcacaaggaggacaataatagctgatcttttgaaatttgaaaaacgtctttagatgaccaagcaaaaagactttaaaaaatggtaatgaaaatggaatgcagctactgcagctaataaaaaattttagatagcaattgttacaaccatatgcctttatagctagacattagaattatgatagcatgagtttatacattctattatttttcctccctttctcatgtttttataaataggtaataaaaaatgttttgcctgccaattgaatgatttcgtagctgaagtagaaacatttaggtttctgtagcattaaattgtgaagacaactggagtggtacttactgaagaaactctctgtatgtcctagaataagaagcaatgatgtgctgcttctgatttttcttgcattttaaattctcagccaacctacagccatgatctttagcacagtgatatcaccatgacttcacagacatggtctagaatctgtacccttacccacatatgaagaataaaattgattaaaggtta

SEQ ID NO: 14 - DNA, 1650 bp, Homo sapiens
gccttttttgcagtctcaggacgggcgctttggagccggccccaggcagcgtgtgtcggtcgcctagtctggagaactagtcctcgactcacggtgagggaatggaccgacacgggtattgtaccgctgagggaaaggagcgggactccggacctccaggagtgcaaggatgatgctgaaaggaataacaaggcttatctctaggatccataagttggaccctgggcgttttttacacatggggacccaggctcgccaaagcattgctgctcacctagataaccaggttccagttgagagtccgagagctatttcccgcaccaatgagaatgacccggccaagcatggggatcagcacgagggtcagcactacaacatctccccccaggatttggagactgtatttccccatggccttcctcctcgctttgtgatgcaggtgaagacattcagtgaagcttgcctgatggtaaggaaaccagccctagaacttctgcattacctgaaaaacaccagttttgcttatccagctatacgatatcttctgtatggagagaagggaacaggaaaaaccctaagtctttgccatgttattcatttctgtgcaaaacaggactggctgatactacatattccagatgctcatctttgggtgaaaaattgtcgggatcttctgcagtccagctacaacaaacagcgctttgatcaacctttagaggcttcaacctggctgaagaatttcaaaactacaaatgagcgcttcctgaaccagataaaagttcaagagaagtatgtctggaataagagagaaagcactgagaaagggagtcctctgggagaagtggttgaacagggcataacacgggtgaggaacgccacagatgcagttggaattgtgctgaaagagctaaagaggcaaagttctttgggtatgtttcacctcctagtggccgtggatggaatcaatgctctttggggaagaaccactctgaaaagagaagataaaagcccgattgcccccgaggaattagcacttgttcacaacttgaggaaaatgatgaaaaatgattggcatggaggcgccattgtgtcggctttgagccagactgggtctctctttaagccccggaaagcctatctgccccaggagttgctgggaaaggaaggatttgatgccctggatccctttattcccatcctggtttccaactataacccaaaggaatttgaaagttgtattcagtattatttggaaaacaattggcttcaacatgagaaagctcctacagaagaagggaaaaaagagctgctgttcctaagtaacgcgaacccctcgctgctggagcggcactgtgcctacctctaagccaagatcacagcatgtgaggaagacagtggacatctgctttatgctggacccagtaagatgaggaagtcgggcagtacacaggaagaggagccaggcccttgtacctatgggattggacaggactgcagttggctctggacctgcattaaaatgggtttcactgtgaatgcgtgacaataagatattcccttgttcctaaaactttatatcagtttattggatgtggtttttcacatttaagataattatggctcttttcctaaaaaataaaatatctttct

SEQ ID NO: 15 - DNA, 1109 bp, Homo sapiens
actggaagaccaggcagcccagctgaaggcagtaagctcggctcacagtcgcaggagagttctggggtacacgggcaaaggggcttgagaaggcccggaggcgaagccgaagagaagcaactgtgccccggagaagagaagctcgcccattccagactgggaaccagctttcagtgaagatggcagggccagaactgttgctcgactccaacatccgcctctgggtggtcctacccatcgttatcatcactttcttcgtaggcatgatccgccactacgtgtccatcctgctgcagagcgacaagaagctcacccaggaacaagtatctgacagtcaagtcctaattcgaagcagagtcctcagggaaaatggaaaatacattcccaaacagtctttcttgacacgaaaatattatttcaacaacccagaggatggatttttcaaaaaaactaaacggaaggtagtgccaccttctcctatgactgatcctactatgttgacagacatgatgaaagggaatgtaacaaatgtcctccctatgattcttattggtggatggatcaacatgacattctcaggctttgtcacaaccaaggtcccatttccactgaccctccgttttaagcctatgttacagcaaggaatcgagctactcacattagatgcatcctgggtgagttctgcatcctggtacttcctcaatgtatttgggcttcggagcatttactctctgattctgggccaagataatgccgctgaccaatcacgaatgatgcaggagcagatgacgggagcagccatggccatgcccgcagacacaaacaaagctttcaagacagagtgggaagctttggagctgacggatcaccagtgggcactagatgatgtcgaagaagagctcatggccaaagacctccacttcgaaggcatgttcaaaaaggaattacagacctctattttttgaagaccgagcagggattagctgtgtcaggaacttggagttgcacttaaccttgtaactttgtttggagctggcacctcttgaaataaaaaggaggatgcacgagctggcaggcatgcaaaaaaaaaaaaaaaaaaaaaaaaaaaa

SEQ ID NO: 16 - DNA, 2818 bp, Homo sapiens
ccctactccgcctctcgggatcctttaagaggcggggcttggctgccagctccgcggcccgggcaaaaggctgggactttactccgggtggcggcgaggacgagtctgtgctccatcagctgccgcacccgccgcctcccgcccccaaaccccatccccgcggttgagccacgatgagcggcagagtcggcgatctgagccccaggcagaaggaggcattggccaagtttcgggagaatgtccaggatgtgctgccggccctgccgaatccagatgactattttctcctgcgttggctccgagccagaagcttcgacctgcagaagtcggaggccatgctccggaagcatgtggagttccgaaagcaaaaggacattgacaacatcattagctggcagcctccagaggtgatccaacagtatctgtcagggggtatgtgtggctatgacctggatggctgcccagtctggtacgacataattggacctctggatgccaagggtctgctgttctcagcctccaaacaggacctgctgaggaccaagatgcgggagtgtgagctgcttctgcaagagtgtgcccaccagaccacaaagttggggaggaaggtggagaccatcaccataatttatgactgcgaggggcttggcctcaagcatctctggaagcctgctgtggaggcctatggagagtttctctgcatgtttgaggaaaattatcccgaaacactgaagcgtctttttgttgttaaagcccccaaactgtttcctgtggcctataacctcatcaaacccttcctgagtgaggacactcgtaagaagatcatggtcctgggagcaaattggaaggaggttttactgaaacatatcagccctgaccaggtgcctgtggagtatgggggcaccatgactgaccctgatggaaaccccaagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataacaccttctcctatagcaggcctggccccctcagtgtctccctgtcaatttctaccccttgtagcagtcattttcgcacaaccctgaagcccaaagaaactgggctggaggacagacctcaggagctttcatttcagttaggcagaggaagagcgactgcagtgggtctccgtgtctatcaaatacctaaggagtccccaggagctggctggccatcgtgataggatctgtctgtcctgtaaactgtgccaacttcacctgtccagggacagcgaagctgggggtggcggggggcatgtaccacagggtggcagcagggaaaaaaattagaaaagggtgaaagattgggacttaacacttcagggaagtcagctgccggggagaaacttgctcctaaatgaacacataagtttagatcgcaatgaggagtagcagggtagctggttgctagagttacggtggggatcagaaactcttccaaacattttagcactgaggctggggtagcttttggcttttcccaggtctcaggaggtggcctgagtcagcacacatcttcccactcggtagacaggctggcctctccctcactttgagactttggcaactcctgggccacacggcctgcctctttgattactaatgattgtcagtgactcagagcttcctgggacttcgggtacccacccgctgttctccatgcaaacaaagcgccagggaaatgacccacagggatcgcagctgcagggagggccagggaggttgggggtgggagtgaatgctaaaagcagatcgtccagtgcccttttcagtgctaccggcctctcaccaagcagtcctccatgtgagcaaccccgagacaaaaatgctaagtgggatcaagagagcagcactcggagagggtgtttgccagtctgagtgtcccgcggtgcccgccaacccgcttcctgactgacctgagcaaggtcttactaagcagtcccatctctgtgggaggcatgcaacgcgtgcagggagttcaggtgccggtcggcgtagccaggcctggaggccccccaggcaggaggccgcccaaaggcggggccggcgtctcgcagactaggggctgggggcggccacagacggcctcgaaaccacagcccttaccccaatcccacgagccccgccaacgaaccacaggtgctgggctttagagaacatgggaaggcggccccagacctggcgggaacgcctttccctcagagccaggccccggccccgtctgggaagctcatcttgcgaagctgagggagctcagggcaaaggccaggctagcgcggaccggaaggggccgaggctgcacgggcctctgccagaacgctcaggacatcccggcctgggtttacaacgctgttaggaaaattaaccaatgaataaagcaacgttcagtgcgca

SEQ ID NO: 17 - DNA, 1475 bp, Homo sapiens
gtcgacgcggccgcgctccgctcccgtgagtaacttggctccgggggctccgctcgcctgcccgcacgccgcccgccacccaggaccgcgccgccggcctccgccgctagcaaacccttccgacggccctcgctgcgcaagccgggacgcctctcccccctccgcccccgccgcggaaagttaagtttgaagaggggggaagaggggaacatggacatgaagaggaggatccacctggagctgaggaaccggaccccggcagctgttcgagaacttgtcttggacaattgcaaatcaaatgatggaaaaattgagggcttaacagctgaatttgtgaacttagagttcctcagtttaataaatgtaggcttgatctcagtttcaaatctccccaagctgcctaaattgaaaaagcttgaactcagtgaaaatagaatctttggaggtctggacatgttagctgaaaaacttccaaatctcacacatctaaacttaagtggaaataaactgaaagatatcagcaccttggaacctttgaaaaagttagaatgtctgaaaagcctggacctctttaactgtgaggttaccaacctgaatgactaccgagagagtgtcttcaagctcctgccccagcttacctacttggatggctatgaccgagaggaccaggaagcacctgactcagatgccgaggtggatggtgtggatgaagaggaggaggacgaagaaggagaagatgaggaagacgaggacgatgaggatggtgaagaagaggagtttgatgaagaagatgatgaagatgaagatgtagaaggggatgaggacgacgatgaagtcagtgaggaggaagaagaatttggacttgatgaagaagatgaagatgaggatgaggatgaagaggaggaagaaggtgggaaaggtgaaaagaggaagagagaaacagatgatgaaggagaagatgattaagaccccagatgacctgcagaaacagaactgttcagtattggttggactgctcatggattttgtagctgtttaaaaaaaaaaaaaaggtagctgtgatacaaaccccaggacacccacccacccaaagagccaaagaatagttcctgtgacattccgccttccttccatgtagtccctcttggtaatctaccaccaagcttgtggacttcaccccaacaaaattgtaagcgttgttaggtttttgtgtaagattcttgctgtagcgtggatagctgtgattggtgagtcaaccgtctgtggctaccagttacactgagattgtaacagcatttttactttctgtacaacaaaaaagctttgtaaataaaatcttaacattttgggtctgttttttcatgctttgctttttaattattattattattttttttacattaggacattttatgtgacaactgccaaaaaagtatttttaagaatttaagcgaaataaacagttactctttggc

SEQ ID NO: 18 - DNA, 841 bp, Homo sapiens
gcaaccactgcagctgggccaagtcgcttagctcttcggtggttgtcacacgtccggaggcctagccgtcgcgtacctaggatgccgcgtggaagccgaagccgcacctcccgcatggcccctccggccagccgggcccctcagatgagagctgcacccaggccagcaccagtcgctcagccaccagcagcggcacccccatctgcagttggctcttctgctgctgcgccccggcagccaggtctgatggcccagatggcaaccactgcagctggcgtggctgtgggctctgctgtggggcacacattgggtcacgccattactgggggcttcagtggaggaagtaatgctgagcctgcgaggcctgacatcacttaccaggagcctcagggaacccagccggcacagcagcagcagccttgcctctatgagatcaaacagtttctggagtgtgcccagaaccagggtgacatcaagctctgtgagggtttcaatgaggtgctgaaacagtgccgacttgcaaacggattggcctaatgaagaagttcaacctggagagatggaaaatcagctctcataactaagttaatttagtataaaaatagaattgatagtgagggtataaagtgtaaccatcagttaaacctctcctgtcattcctagcttccttgcttcagaattgaaatggaagtgggggtgtccctactctgtagaatctgggactgggcaaatgtttgtgtggcctccttaaactagctgttatgttatgattttattctttgtgagttaattagaataaagtcattttcttacaaaaaaaaaaaaaaaaaaaaaaaaaaaa

SEQ ID NO: 19 - DNA, 1486 bp, Homo sapiens
gggctcgtcagatatattaattttacacttcagttttgattggtgagaaagtacccattctcttcaaataatcaaagataattattattttgttttgtttttggaatcaacagggaggcgcaaagtataaagttgctgctaacatatatacatatacatccatattttataagggtgtctatgtatatatagacagtgtgtccacacaaaaaatagatacagttatcagtcagtcagttcttccatgatttagtttttttaaacgtagaaaagctattgtaaacgtctctttccatttattcttaattttttgacatattggtatttctttaaagggaaatgaggaatgcacatcagtgattgattgtcaaacctcaccccctgatttcctacctaatctacccccacctaaccaatcaatcacatccacaaattgttttgtttgtttgttagtcaggcttccaacagagttcaatatttctaacactctagtgcaataaaaattattattaaatagctaagaggtgtgcatgtgggaaaggtcagtgcatatccctttaggaggggagaatgttgtaatatatcagctatcgagttgtttaaaaaaagtgtattcaatcgtatattgtctatagtatgtgctatgaaatttgcatttatgatatgtaacaggggcaaagccaaattcatgttactctgttcagtcagaaacattttgtggcatacagcattcctgggaagtgctgtactttgtttcgttttggttttagttttgcatttagagtgccttataattgatgcctattttaatagcatttctttttagcttttggttcgtatttccattcactgttcgtatctgttactttctattaaagcattatctgtttaccacatgtacaaaaactctttgaataatatgcattcctagttttcagccaagacggggatgttagtgattgtaccagcccaaagcacttggataatcagggcccttcttccttttataatcaatcatcaacatcagaaaaagctacttgttttatttatattcccttccaaatccgctctggaacatgcagtaactgcaccaaacttattttagtaacaaatatcattggcaactttggaatatatttgatattccattaggatttttctaaaaggggaaataaactatatatatatatgtatcttacccccaattcttccaacagaatttctataggaagccatggatgatggcataagtttgccacatattacatgattttaaataatcctcaaaatacccaaggaactcttaaagagttttggtatgagtatactactttggtttaattttagcttcatggatgttctgcatggaaggatttttgttttccacattttcccattgctagcagagtgaaatccaagagaccaaacatttgcaagcattgtatttgagcacttttgtaaaaaacaaaaaaaaaaaaaaaaaaaa

SEQ ID NO: 20 - DNA, 16 bp, Bacteriophage M13mp18
gtaaaacgacggccag

SEQ ID NO: 21 - DNA, 17 bp, Bacteriophage M13mp18
caggaaacagctatgac

SEQ ID NO: 22 - DNA, 30 bp, Homo sapiens
caggtgaatttcaaaggaggatttactcac

SEQ ID NO: 23 - DNA, 30 bp, Homo sapiens
gtgagtaaatcctcctttgaaattcacctg

SEQ ID NO: 24 - DNA, 23 bp, Homo sapiens
gcaagccaggaagagtcgtcacg

SEQ ID NO: 25 - DNA, 25 bp, Homo sapiens
tgccaggaaacttcttgcttgatgc

SEQ ID NO: 26 - DNA, 26 bp, Homo sapiens
agtaaccagttgagatgaagcacgtc

SEQ ID NO: 27 - DNA, 28 bp, Homo sapiens
cagaagagcaacaagaatggtatcctgc

SEQ ID NO: 28 - DNA, 25 bp, Homo sapiens
aacttgagttctatttaccttgcac

SEQ ID NO: 29 - DNA, 21 bp, Homo sapiens
ttgcttgggtcatctaaagac

SEQ ID NO: 30 - DNA, 20 bp, Homo sapiens
actcacgtgcaaggatgatg

SEQ ID NO: 31 - DNA, 20 bp, Homo sapiens
agctctcggactctcaactg

SEQ ID NO: 32 - DNA, 26 bp, Homo sapiens
cttctcctatgactgatcctactatg

SEQ ID NO: 33 - DNA, 21 bp, Homo sapiens
caggatgcagaactcaccctg

SEQ ID NO: 34 - DNA, 21 bp, Homo sapiens
gcagatttcccgtggctcctc

SEQ ID NO: 35 - DNA, 22 bp, Homo sapiens
gttgggcagcacctctgtcatc

SEQ ID NO: 36 - DNA, 22 bp, Homo sapiens
ctgtgacattccgccttccttc

SEQ ID NO: 37 - DNA, 23 bp, Homo sapiens
ccacgctactgcaagaatcttac

SEQ ID NO: 38 - DNA, 23 bp, Homo sapiens
agaagttcaacctggagagatgg

SEQ ID NO: 39 - DNA, 24 bp, Homo sapiens
caaggaagctaggaatgacaggag

SEQ ID NO: 40 - DNA, 24 bp, Homo sapiens
gcaaagccaaattcatgttactct

SEQ ID NO: 41 - DNA, 27 bp, Homo sapiens
cagatacgaacagtgaatggaaatacg

SEQ ID NO: 42 - DNA, 24 bp, Homo sapiens
gccacaggttgaacacttaatttg

SEQ ID NO: 43 - DNA, 22 bp, Homo sapiens
aggaagagtcgtcacgagaacc

SEQ ID NO: 44 - DNA, 25 bp, Homo sapiens
ataatgctgtgcttagtttattgcc

SEQ ID NO: 45 - DNA, 21 bp, Homo sapiens
gatcgtggacatttcaacctc

SEQ ID NO: 46 - DNA, 20 bp, Homo sapiens
tcttgcttgatgctttggtc

SEQ ID NO: 47 - protein, 1254 aa, Mus musculus
MPGGSVNITCVAVGSPMPYVKWMLGAEDLTPEDDMPIGRNVLELNDVRQSANYTCVAMSTLGVIEAIAQITVKALPKPPGTPVVTESTATSITLTWDSGNPEPVSYYIIQHKPKNSEEPYKEIDGIATTRYSVAGLSPYSDYEFRVVAVNNIGRGPASEPVLTQTSEQAPSSAPRDVQARMLSSTTILVQWKEPEEPNGQIQGYRVYYTMDPTQHVNNWMKHNVADSQITTIGNLVPQKTYSVKVLAFTSIGDGPLSSDIQVITQTGVPGQPLNFKAEPESETSILLSWTPPRSDTIASYELVYRDGDQGEEQRITIEPGTSYRLQGLKPNSLYYFRLSARSPQGLGASTAEISARTMQSMFAKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPDVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMDHAESKMYATSPYSDPVVSMDLDPQPITDEEEGLIWVVGPVLAVVFIICIVIAILLYKRKRAESESRKSSLPNSKEVPSHHPTDPVELRRLNFQTPGMASHPPIPILELADHIERLKANDNLKFSQEYESIDPGQQFTWEHSNLEVNKPKNRYANVIAYDHSRVLLSAIEGIPGSDYVNANYIDGYRKQNAYIATQGSLPETFGDFWRMIWEQRSATVVMMTKLEERSRVKCDQYWPSRGTETHGLVQVTLLDTVELATYCVRTFALYKNGSSEKREVRQFQFTAWPDHGVPEHPTPFLAFLRRVKTCNPPDAGPMVVHCSAGVGRTGCFIVIDAMLERIKHEKTVDIYGHVTLMRAQRNYMVQTEDQYIFIHDALLEAVTCGNTEVPARNLYAYIQKLTQIETGENVTGMELEFKRLASSKAHTSRFISANLPCNKFKNRLVNIMPYESTRVCLQPIRGVEGSDYINASFLDGYRQQKAYIATQGPLAETTEDFWRMLWEHNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDQYQFCYRAALEYLGSFDHYAT

SEQ ID NO: 48 - DNA, 21 bp, homo sapiens
aatctgcaagccaggaagagt

SEQ ID NO: 49 - DNA, 27 bp, homo sapiens
tctagtttcagttttgatgatattttg

SEQ ID NO: 50 - RNA, 19 nt, homo sapiens
ucugcaagccaggaagagu

SEQ ID NO: 51 - RNA, 19 nt, homo sapiens
acucuuccuggcuugcaga

SEQ ID NO: 52 - RNA, 19 nt, homo sapiens
ccuccagaacugugaucca

SEQ ID NO: 53 - RNA, 19 nt, homo sapiens
uggaucacaguucuggagg

SEQ ID NO: 54 - RNA, 19 nt, Homo sapiens
cuacaaaugagcgcuuccu

SEQ ID NO: 55 - RNA, 19 nt, Homo sapiens
aggaagcgcucauuuguag

SEQ ID NO: 56 - DNA, 20 bp, Homo sapiens
ccacatcgctcagacaccat

SEQ ID NO: 57 - DNA, 17 bp, Homo sapiens
accaggcgcccaatacg

SEQ ID NO: 58 - DNA, 28 bp, Homo sapiens
caaatccgttgactccgaccttcacctt

SEQ ID NO: 59 - DNA, 20 bp, Homo sapiens
aaggccaaccgcgagaagat

SEQ ID NO: 60 - DNA, 20 bp, Homo sapiens
gtcaccggagtccatcacga

SEQ ID NO: 61 - DNA, 32 bp, Homo sapiens
ccatgtacgttgctatccaggctgtgctatcc

SEQ ID NO: 62 - DNA, 25 bp, Homo sapiens
caactgggacgacatggagaaaatc

SEQ ID NO: 63 - DNA, 22 bp, Homo sapiens
catggctggggtgttgaaggtc

SEQ ID NO: 64 - DNA, 23 bp, Homo sapiens
actctcacctcccatgttgctca

SEQ ID NO: 65 - DNA, 23 bp, Homo sapiens
gctatccgtgcactcctgttctg

SEQ ID NO: 66 - DNA, 21 bp, Homo sapiens
atctgcaagccaggaagagtc

SEQ ID NO: 67 - DNA, 22 bp, Homo sapiens
cttgcttgatgctttggtctgt

SEQ ID NO: 68 - DNA, 30 bp, Homo sapiens
ccagaccatgcaggaactctgatcgtggac

SEQ ID NO: 69 - DNA, 21 bp, Homo sapiens
atgccctggatccctttattc

SEQ ID NO: 70 - DNA, 23 bp, Homo sapiens
tcatcccgacttcctcatcttac

SEQ ID NO: 71 - DNA, 24 bp, Homo sapiens
aactcccttattacactatccatt

SEQ ID NO: 72 - DNA, 24 bp, Homo sapiens
gtgttatgaggaaaagattaggga

SEQ ID NO: 73 - DNA, 30 bp, Homo sapiens
tgcagccaggagaagcaagagaacagaaat

(19) United States
(12) Patent Application Publication
(10) Pub. No.: US 2005/0262577 A1
(43) Pub. Date: Nov. 24, 2005

(54) Polypeptides and nucleic acids encoding these and their use for the prevention, diagnosis or treatment of liver disorders and epithelial cancer

(76) Inventors: Christian Guelly, Graz (AT); Charles Buck, West Lafayette, IN (US); Kurt Zatloukal, Graz (AT)

Correspondence Address: WENDEROTH, LIND & PONACK, L.L.P., 2033 K STREET N. W., SUITE 800, WASHINGTON, DC, 20006-1021, US

(21) Appl. No.: 10/528,031

(22) PCT Filed: Sep. 23, 2003
(86) PCT No.: PCT/EP03/10564

§ 371 Date: Mar. 16, 2005

(30) Foreign Application Priority Data
Sep. 27, 2002 (EP) 02021696.6
Oct. 3, 2002 (US) 60415913

(51) Int. Cl. A01K 67/00; C12Q 1/68; C07H 21/04; C12N 15/09; C07K 14/82; C07K 16/30

(52) U.S. Cl. 800/8; 435/6; 435/69.1; 435/320.1; 435/325; 530/350; 530/388.8; 536/23.5

(57) ABSTRACT

The invention relates to polypeptides and nucleic acids encoding these and to their use for the diagnosis, prevention and/or treatment of liver disorders and neoplastic disorders, especially cancer of the liver and other epithelial tissues, benign liver neoplasms such as adenoma and other proliferative liver disorders such as focal nodular hyperplasia (FNH) and cirrhosis. The invention further relates to methods of diagnosing and treating these disorders.

### TECHNICAL FIELD

The invention relates to polypeptides and nucleic acids encoding these and to their use for the diagnosis, prevention and/or treatment of liver disorders and neoplastic disorders, especially cancer of the liver and other epithelial tissues, benign liver neoplasms such as adenoma and other proliferative liver disorders such as focal nodular hyperplasia (FNH) and cirrhosis. The invention further relates to methods of diagnosing and treating these disorders.

### BACKGROUND ART

The development of cancer in general is characterized by genetic mutations that alter activity of important cellular pathways including, for example, proliferation, apoptosis (cell death), response to stress and epithelial/stroma interactions. It is increasingly recognized that identification of nucleic acids that are deregulated in cancer can provide important new insight into the mechanisms of neoplastic transformation. Identification of deregulated nucleic acid expression in precancerous stages, such as macro regenerative nodules and the “large” and “small” cell change in liver cancer, provide understanding of early events in malignant transformation. Similarly, identification of deregulated gene expression in disorders characterized by tissue proliferation and remodeling, such as FNH and cirrhosis in the liver may distinguish nucleic acids involved in proliferation and malignant transformation. Together such deregulated nucleic acids and the encoded gene products have potential as new diagnostic markers for cancer. Moreover, the products of these deregulated nucleic acids per se are targets for therapeutic intervention in the prevention and/or treatment of these disorders in human patients.

The liver plays a vital role in the metabolism of proteins, lipids, carbohydrates, nucleic acids and vitamins. There are numerous disorders effecting the liver that cannot be diagnosed, prevented or treated effectively, such as hepatocellular carcinoma (HCC). Examination of HCC is particularly well suited for the identification of deregulated gene expression in cancer. This is because tissue samples of HCC can be obtained from surgically resected tumors and the tumors are well circumscribed solid structures with little stromal tissue.

Furthermore, as indicated above, there is the possibility for comparative analyses of benign and malignant tumors as well as cirrhosis, a non-neoplastic condition. If the limitations in the art of identifying differentially expressed genes associated with liver disorders could be overcome, this comparative approach may enable identification of deregulated nucleic acids specifically involved in the processes of cellular proliferation and tissue remodeling in a mature organ (e.g., in cirrhosis) as well as the identification and discrimination of gene expression alterations associated with hyperplasia (such as FNH) and with benign and malignant neoplasms (e.g., adenoma and HCC). In HCC there is an urgent need for new and better diagnostic and therapeutic capabilities. Deregulated genes in liver cancer may also be highly relevant to other cancers of the gastrointestinal tract and indeed with other carcinomas (epithelial derived cancers) as these tissues share a common embryological origin.

On a global basis, hepatocellular carcinoma (HCC) belongs to the most common malignant tumors accounting for about 1 million deaths/year (Ishak et al, 1999. Atlas of Tumor Pathology. Fascicle 31. Armed Forces Institute of Pathology, Washington, D.C.).

Definitive diagnosis of neoplastic liver disorders such as HCC and many other tumors relies upon histopathological evaluation of biopsy specimens. This invasive surgical procedure is generally not undertaken until symptoms appear and the disease is then most often in advanced stages, thereby limiting therapeutic intervention options. Thus there is a need to improve diagnostics and methods of diagnosis. In addition, early diagnosis is crucial but hampered by late onset or even a lack of specific clinical symptoms. At diagnosis most HCC tumors are no longer amenable to surgical resection (except encapsulated tumors or the fibrolamellar variants) (Chen and Jeng, 1997, J. Gastroenterol. Hepatol. 12: 329-34); moreover, they are highly resistant to cytostatic therapy (Kawata et al., 2001 Br. J. Cancer 84:886-91). Overall, death usually occurs within 1 year after diagnosis. Thus, markers for early detection, prognostic indicators, and effective prevention and/or treatment regimens for HCC are highly desirable in this field.

In contrast, unlike the well-studied situation in colorectal cancer, liver adenoma may not represent a precursor lesion of HCC. Similarly, although cirrhosis and hepatitis viral infections are clearly risk factors for HCC, these conditions are not prerequisite for the development of HCC. Certain liver lesions may represent HCC prestages such as macro regenerative nodular hyperplasia, but this is not yet confirmed (Shortell and Schwartz, 1991, Surg Gynecol Obstet. 173:426-31; Anthony, P. in MacSween et al, eds. Pathology of the Liver. 2001, Churchill Livingstone, Edinburgh). Although these disorders are diagnosed by histopathological investigation of liver resections and liver biopsies, no efficient method exists for earlier or non-invasive detection of these conditions. Again, there is immediate need for diagnostic and prognostic markers for these neoplasms and for non-invasive detection of these disorders.

Within the past decade, several technologies have made it possible to monitor the expression level of a large number of transcripts within a cell at any one time (see, e.g., Schena et al., 1995, Science 270:467-470; Lockhart et al., 1996, Nature Biotechnology 14:1675-1680; Blanchard et al., 1996, Nature Biotechnology 14, 1649; 1996, U.S. Pat. No. 5,569,588). Transcript array technology has been utilized for the identification of genes that are up regulated or down regulated in various disordered states. Several recent studies have utilized this technology to examine changes in gene expression in HCC. These studies have variously revealed deregulation (i.e., over- and underexpression) of genes encoding liver specific proteins in HCC cell lines and HCC tissues relative to controls. Moreover the studies revealed genes essential for cell cycle control, stress response, apoptosis, lipid metabolism, cell-cell-interaction, DNA repair and cytokine and growth factor production (Graveel et al, 2001, Oncogene 20:2704-12; Kawai et al, 2001, Hepatology 33:676-91; Lau et al, 2000, Oncol. Res. 12:59-69; Nagai et al, 1998, Cancer 82:454-61; Okabe et al, 2001, Cancer Res 61:2129-37; Salvucci et al, 1999, Oncogene 18:181-187; Shirota et al, 2001, Hepatology 33:832-40; Tackels-Home et al, 2001, Cancer 92: 395-405; Wu et al, 2001, Oncogene 20:2674-3682; Xu et al, 2001, Cancer Res. 61:3176-81). However, there is little concordance in the gene expression patterns reported in these studies that may be due to differences in experimental design and/or to the heterogeneity of HCC tissue per se. Moreover, the etiologies of these HCCs are an important factor. Chronic hepatitis B and C virus infections are the major causes of HCC but damage from alcohol and chronic liver metabolic disorders are also recognized to result in HCC and the mechanisms responsible for development of a tumor from these different etiologies are likely to differ. Taken together, until now no satisfactory diagnostics and methods of diagnosing have been developed in order to be able to intervene in liver disorders.

The same applies to the therapy of liver disorders, and epithelial cancers. For HCC for instance, there is no effective therapeutic option except resection and transplantation but these approaches are only applicable in early stages of HCC, limited by the access to donor livers, and associataed with severe risks for the patient. In addition, these approaches are extremely expensive. These cancers respond very poorly to chemotherapeutics, most likely due the normal liver function in detoxification and export of harmful compounds. Several other therapeutic options, such as chemoembolization, cryotherapy and ethanol injection are still in an experimental phase and the efficacy of these is not established. Surgical intervention remains the best treatment option but it is not possible to define with precision the extent of the tumor. This invasive procedure therefore, is suboptimal from the perspective of treatment. Furthermore, the lack of early diagnostics for specific liver dysfunctions leads most often to advanced progression of the disease that further confounds therapeutic options and dramatically increases patient mortality from these diseases (Jansen P. L., 1999, Neth. J. Med. 55:287-292). Thus until now no satisfactory therapies have been developed in order to be able to intervene in liver disorders, and other epithelial cancers. Furthermore, in the state of the art, recognition of the different subtypes of liver disorders such as HCC precursor lesions, benign liver neoplasms, and metabolic liver diseases such as alcoholic liver disease and cirrhosis, as revealed by differential gene expression, have not been disclosed. A summary of the key disease features of some of the disorders evaluated in the invention is provided in Table 1.

TABLE 1
Diseases Features

| | | Tissue | | | Transformation/ |
|---|---|---|---|---|---|
| DIS- | Cellular | remod- | Clonal cell | Neo- | Malignant |
| ORDER | proliferation | eling | expansion | plasia | potential |
| Cirrhosis | + | + | | | |
| FNH | + | + | +/− | | |
| Adenoma | + | + | + | + | |
| HCC | + | + | + | + | + |

### SUMMARY OF THE INVENTION

The invention relates to polypeptides and nucleic acids encoding these and their use for the diagnosis, prevention and/or treatment of liver disorders, especially of hepatocellular carcinoma (HCC), and epithelial cancers, pre-cancerous liver lesions, benign neoplasms such as adenoma, and other proliferative liver disorders such as focal nodular hyperplasia (FNH) and cirrhosis that overcome the limitations present in the art. The invention also relates to vectors and cells comprising such nucleic acids, and to antibodies or antibody fragments directed against said polypeptides and nucleic acids.

The invention further relates to methods of diagnosing and treating these disorders. The evaluation of multiple disorders with overlapping but distinct morphological and clinical features provides new information for identification and discrimination and ultimately new therapeutic strategies for these disorders according to invention.

### DETAILED DESCRIPTION

A unique approach employed in this invention utilizes discrete, pathologist-confirmed liver cancer pathologies for production of disease specific cDNA libraries enriched in genes specifically up- and down-regulated in HCC compared with a pool of non-neoplastic human livers. The library is a genome-wide representation of deregulated gene expression in HCC and therefore includes all potential HCC deregulated genes. Repetitive hybridization to these library clones with labeled expressed nucleic acids from many additional discrete, pathologist-confirmed liver cancer samples (HCCs) and non-malignant liver lesions indicated nucleic acids highly deregulated in HCC. The surprising finding is that this approach provides deregulated nucleic acids that had not previously been identified as well as many deregulated nucleic acids that were not before associated with HCC, the elevated expression of which can also be associated with other neoplasms. These HCC deregulated genes and proteins are the subject of this invention.

The screening and verification strategy is already inventive per se owing to the elaborate and defined choice of parameters. Identification of differentially expressed genes according to the invention relies upon histopathologically distinguished liver disease tissue for comparison of gene expression changes in disorders of the human liver. Non-diseased reference liver samples for the experiments are also diagnostically confirmed.

The object of the invention is solved by a method of diagnosis of a liver disorder, liver cancer and/or epithelial cancer, wherein at least one compound selected from the group consisting of a polypeptide according to the sequence of SEQ ID 1 to SEQ ID 9 and/or SEQ ID 47 (Table 2), a functional variant thereof, a nucleic acid encoding one of the aforementioned polypeptides, a variant of one of the aforementioned nucleic acids, an antibody or a fragment of the antibody directed against one of the aforementioned polypeptides, or variants thereof, is identified in the sample of a patient and compared with at least one compound of a reference library or of a reference sample.

The object of the invention is also solved by a method of treating a patient suffering from a liver disorder or an epithelial cancer, wherein at least one component selected from the group consisting of a polypeptide according SEQ ID 1 to 9 and/or SEQ ID 47, a functional variant of one of the aforementioned polypeptides, a nucleic acid encoding one of the aforementioned polypeptides, or a functional variant thereof, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of one of the aforementioned antibodies directed against one of the aforementioned polypeptides or against a functional variant thereof, a vector comprising a nucleic acid coding for one of the aforementioned antibodies, a vector comprising a nucleic acid coding for one of the aforementioned antibody fragments, a cell comprising the vector comprising a nucleic acid coding for one of the aforementioned antibodies, and a cell comprising the vector comprising a nucleic acid coding for one of the aforementioned antibody fragments, is administered to the patient in need of a the treatment in a therapeutically effective amount.

In another aspect of the invention it is provided a pharmaceutical composition comprising at least one compound selected from the group consisting of a polypeptide according to the invention, a functional variant thereof, a nucleic acid encoding the polypeptide, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody directed against one of the aforementioned polypeptides, an antibody directed against a functional variant of one of the aforementioned polypeptides, a fragment of one of the aforementioned antibodies, a vector comprising a nucleic acid coding for one of the aforementioned antibodies, a vector comprising a nucleic acid coding for one of the aforementioned antibody fragments, a cell comprising the vector comprising a nucleic acid coding for one of the aforementioned antibodies, and a cell comprising the vector comprising a nucleic acid coding for one of the aforementioned antibody fragments, is administered to the patient in need of a the treatment in a therapeutically effective amount.

The accession numbers of the polypeptides according to the invention and their cDNAs are shown in Table 2.

TABLE 2
Nucleic acids and polypeptides with their respective SEQ ID numbers
and accession numbers from the GenBank database.

| | Polypeptide | Accession | DNA | |
|---|---|---|---|---|
| Molecule | (SEQ ID) | number | (SEQ ID) | Accession number |
| OBcl1 | 1 | NP_443111 | 10 | AL833272 |
| OBcl5 | 2 | Novel | 11 | Novel |
| IK2 | 3 | NP_079436 | 12 | NM_025160 |
| IK5 | 4 | NP_006398 | 13 | NM_006407 |
| DAP3 | 5 | NP_387506 | 14 | NM_033657 |
| LOC5 | 6 | NP_060917 | 15 | NM_018447 |
| SEC14L2 | 7 | NP_036561 | 16 | NM_012429 |
| SSP29 | 8 | NP_006392 | 17 | NM_006401 |
| HS16 | 9 | NP_057223 | 18 | NM_016139 |
| IK3 | 47 | XM_131462 | 19 | AL049338 |

A subset of these nucleic acids and polypeptides according to the invention have been shown by RT-PCR analysis to be specifically expressed or deregulated in other cancers of epithelial origin and preferably not in corresponding normal human tissue(s). These nucleic acids preferably include SEQ ID Nos. 11 to 16 and 19 (provided in Table 6 and FIG. 3). Deregulated nucleic acids in liver cancer may preferably be highly relevant to other cancers of the gastrointestinal tract as these tissues share a common embryological origin. Consequently, these nucleic acids and the encoded polypeptides may preferably be similarly utilized for diagnostics methods of diagnosis, pharmaceutical compositions and methods of prevention and/or treatment of these epithelial cancers.

The polypeptides and nucleic acids according to the invention have in common that they are differentially expressed in a sample isolated from a patient suffering from a disorder according to the invention compared to a reference sample. The regulation of the polypeptides and nucleic acids according to the invention is essential for the pathologic process and which are thus in a direct or indirect relationship with diagnosis, prevention and/or treatment of disorders according to the invention. The polypeptides and the nucleic acids according to the invention do not belong to the targets known until now such that suprising and completely novel approaches for diagnosis and therapy result from this invention.

Generally, the analysis of differentially expressed genes in tissues is less likely to result in errors in the form of artifactual false-positive clones than the analysis of cell culture systems. In addition to the fact that existing cell culture systems cannot adequately simulate the complexity of pathological processes in the tissue, the variations in cell behavior in the culture environment lead to nucleic acid and polypeptide expression patterns with questionable relation to the actual pathologic state. These problems may be less pronounced by an approach that utilizes gene expression in normal and diseased human tissue but again multiple variables confound clear identification of differential gene expression that is directly relevant to disease. For example, differentially expressed nucleic acids may result from inter-individual differences, metabolic state and/or clinical treatment paradigm. Further, large scale gene expression studies using cDNA microarrays do not indicate the cellular source of variation in gene expression. In addition, a differential gene expression study including all or most genes produces a very large volume of data that confounds identification of key disease-associated gene expression changes. Consequently, an approach that includes large scale profiling of gene expression from tissue from liver disorders that are defined only generally (as for example, ‘liver tumors’) is unlikely to illuminate key genes involved in the disease process and it is these key genes that represent best targets for diagnostics and therapeutic intervention.

On account of these difficulties, the success of the screening is significantly dependent on the choice of the experimental parameters. While the methods used are based on established procedures, the screening and verification strategy is already inventive per se owing to the elaborate and defined choice of parameters. A unique approach employed in this invention utilizes discrete, pathologist-confirmed liver cancer pathologies for production of disease specific cDNA libraries enriched in nucleic acids specifically up- and down-regulated in HCC compared with a pool of non-neoplastic human livers. Non-diseased reference liver samples for the experiments are also diagnostically confirmed and pooled from 3 independent samples to reduce detection of false positives resulting from inter-individual variations. Nucleic acids commonly expressed at similar levels in the reference liver pool and in diseased liver (i.e., HCC) are removed by the generation of subtractive suppressive hybridization (SSH) cDNA libraries (Diatchenko et al., 1996, Proc. Natl. Acad. Sci. USA 93:6025-6030). These cDNAs are highly enriched for nucleic acids both up- and down-regulated in HCC but do not represent those that are not differentially expressed. Each of several thousand SSH clones were amplified by the polymerase chain reaction (PCR) and affixed to glass slides in custom cDNA microarrays. RNA from additional pathologist-confirmed liver disorders is converted to fluorescently-labeled cDNA for competitive hybridization with the pooled non-diseased liver RNA on the microarrays. The resulting ratio of hybridization intensity reveals nucleic acids specifically deregulated in liver disorders. In addition to providing a pool of candidate cDNAs highly enriched for differentially expressed genes, the SSH library represents on a genome-wide scale most if not all differentially expressed genes with far fewer clones than in standard cDNA libraries. This feature thereby focuses on nucleic acids specifically deregulated in disease. The SSH libraries generated in this invention include cDNA clones from nucleic acids that are essentially not expressed in normal liver and thereby not represented in conventional cDNA libraries or on genome-scale cDNA microarrays.

Over expression of the sequences according to the invention in liver disorder tissue compared to normal liver is confirmed by independent analysis of RNA levels with sequence-specific quantitative RT-PCR (Q-PCR) (FIG. 2). In these verification experiments, PCR product corresponding to the cellular RNA levels of the sequences according to the invention are monitored by fluorescent detection of the specific PCR product. The fluorescent signal is provided either by a sequence specific hydrolysis probe oligonucleotide (primer) in the TaqMan procedure or by a fluorscent double stranded DNA binding dye such as SYBR green. Levels of PCR products corresponding to the sequences according to the invention are normalized for experimental variability by comparison with the levels of ‘housekeeping’ genes including glyceraldehyde dehydrogenase (GAPDH) and β-actin, which are considered relatively invariant in disease or following experimental manipulations. These Q-PCR procedures are also in use to measure levels of gene expression in experimental situations such as in the case when the level of a sequence according to the invention is experimentally decreased with small interfering RNA oligonucleotides (FIG. 6, Table 10). The reference gene primers used for TaqMan Q-PCR analyses are GAPDH-p1, SEQ ID 56; GAPDH-p2, SEQ ID 57; GAPDH-p3, SEQ ID 58; bActin-p1, SEQ ID 59; bActin-p2, SEQ ID 60; and bActin-p3, SEQ ID 61. The reference gene primers used for SYBR Green analyses are bActin-p4, SEQ ID 62; and bActin-p5, SEQ ID 63. The determination of RNA levels relative to these housekeeping genes in Q-PCR experiments was performed according to the method of Pffafl (Nucleic Acids Research (2001) May 1, 29(9):e45). These techniques are well known to a person skilled in the art.

Furthermore, expression of HCC deregulated genes according to this invention correlates with proliferation of hepatoma cells (Hep3B) following 8 hours and 12 hours serum stimulation of quiescent cells (FIG. 8). This finding supports the suggestion that over-expression of the sequences according to the invention is functionally significant for proliferative liver disorders such as liver cancer.

Compared to the state of the art, these polypeptides and nucleic acids surprisingly allow improved, more sensitive, earlier, faster, and/or non-invasive diagnosis of the liver disorders and/or epithelial cancers. The nucleic acids and polypeptides according to the invention can be utilized for the diagnosis, prevention and treatment of liver disorders, and epithelial cancers.

The present invention relates to a polypeptide comprising a sequence according to the SEQ ID 2, or a functional variant thereof. The invention also relates to a nucleic acid coding for the polypeptide, or a functional variant thereof, in particular to the nucleic acid according to the SEQ ID 11 and variants thereof.

In preferred embodiment the polypeptide consists of the sequence according to the SEQ ID 2. In another preferred embodiment the nucleic acid consists of the sequence according to SEQ ID 11.

Compared to the state of the art, these polypeptides and nucleic acids surprisingly allow improved, more sensitive, earlier, faster, and/or non-invasive diagnosis of the liver disorders and/or epithelial cancers.

In another aspect of the invention the invention relates to the use of at least one polypeptide according SEQ ID 1 to 9 and/or SEQ ID 47, a functional variant of the polypeptide, a nucleic acid encoding one of the aforementioned polypeptides, a nucleic acid encoding the functional variant, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody directed against one of the aforementioned polypeptides, an antibody directed against a functional variant of one of the aforementioned polypeptides, a fragment of one of the aforementioned antibodies, a vector comprising a nucleic acid coding for one of the aforementioned antibodies, a vector comprising a nucleic acid coding for one of the aforementioned antibody fragments, a cell comprising the vector comprising a nucleic acid coding for one of the aforementioned antibodies, and/or at least one cell comprising the vector comprising a nucleic acid coding for one of the aforementioned antibody fragments, for the diagnosis, prevention and/or treatment of disorders according to the invention. Further embodiments of the invention are described in detail below.

When compared to the state of the art of therapy of liver disorders, and/or epithelial cancers the use of these components surprisingly provide an improved, sustained and/or more effective diagnosis, prevention and/or treatment of disorders according to the invention.

The term “polypeptide” refers to the full length of the polypeptide according to the invention. In a preferred embodiment the term “polypeptide” also includes isolated polypeptides and polypeptides that are prepared by recombinant methods, e.g. by isolation and purification from a sample, by screening a library and by protein synthesis by conventional methods, all of these methods being generally known to the person skilled in the art. Preferably, the entire polypeptide or parts thereof can be synthesized, for example, with the aid of the conventional synthesis such as the Merrifield technique. In another preferred embodiment, parts of the polypeptides according to the invention can be utilized to obtain antisera or specific monoclonal antibodies, which may be used to screen suitable gene libraries prepared to express the encoded protein sequences in order to identify further functional variants of the polypeptides according to the invention.

The term “polypeptide according to the invention” refers to the polypeptides according to SEQ ID 1 to SEQ ID 9 and/or SEQ ID 47 (Table 2).

The term “functional variants” of a polypeptide within the meaning of the present invention refers to polypeptides which have a sequence homology, in particular a sequence identity, of about 70%, preferably about 80%, in particular about 90%, especially about 95%, most preferred of 98% with the polypeptide having the amino acid sequence according to one of SEQ ID 1 to SEQ ID 9 and/or SEQ ID 47. Such functional variants are, for example, the polypeptides homologous to a polypeptide according to the invention, which originate from organisms other than human, preferably from non-human mammals such as, for example mouse, rats, monkeys and pigs. Other examples of functional variants are polypeptides that are encoded by different alleles of the gene, in different individuals, in different organs of an organism or in different developmental phases. Functional variants, for example, also include polypeptides that are encoded by a nucleic acid which is isolated from non-liver-tissue, e.g. embryonic tissue, but after expression in a cell involved in liver disorders have the designated functions. Functional variants preferably also include naturally occurring or synthetic mutations, particularly mutations that quantitatively alter the activity of the peptides encoded by these sequences. Further, such variants may preferably arise from differential splicing of the encoding gene.

“Functional variants” refer to polypeptides that have essentially the same biological funtion(s) as the corresponding polypeptide according to the invention. Such biological function can be assayed in a functional assay.

In order to test whether a candidate polypeptide is a functional variant of a polypeptide according the invention, the candidate polypeptide can be analyzed in a functional assay generally known to the person skilled in the art, which assay is suitable to assay the biological function of the corresponding polypeptide according to the invention. Such functional assay comprise for example cell culture systems; the generation of mice in which the genes are deleted (“knocked out”) or mice that are transgenic for gene encoding the candidate polypeptide; enzymatic assays, etc. If the candidate polypeptide demonstrates or directly interferes with essentially the same biological function as the corresponding polypeptide according to the invention, the candidate polypeptide is a functional variant of the corresponding polypeptide, provided that the candidate polypeptide fulfills the requirements on the level of % sequence identity mentioned above.

Furthermore, the term “functional variant” encompasses polypeptides that are preferably differentially expressed in patients suffering from liver disorders, or other epithelial cancers relative to a reference sample or a reference library, including polypeptides expressed from mutated genes or from genes differentially spliced, provided that the candidate functional variant polypeptide fulfills the criteria of a functional variant on the level of % sequence identity. Such expression analysis can be carried out by methods generally known to the person skilled in the art.

“Functional variants” of the polypeptide can also be parts of the polypeptide according to the invention with a length of at least from about 7 to about 1000 amino acids, preferably of at least 10 amino acids, more preferably at least 20, most preferred at least 50, for example at least 100, for example at least 200, for example at least 300, for example at least 400, for example at least 500, for example at least 600 amino acids provided that they have essentially the same biological function(s) as the corresponding polypeptide according to the invention. Also included are deletions of the polypeptides according to the invention, in the range from about 1-30, preferably from about 1-15, in particular from about 1-5 amino acids provided that they have essentially the same biological function(s) as the corresponding polypeptide according to the invention. For example, the first amino acid methionine can be absent without the function of the polypeptide being significantly altered. Also, post-translational modifications, for example lipid anchors or phosphoryl groups may be present or absent in variants.

“Sequence identity” refers to the degree of identity (% identity) of two sequences, that in the case of polypeptides can be determined by means of for example BLASTP 2.0.1 and in the case of nucleic acids by means of for example BLASTN 2.014, wherein the Filter is set off and BLOSUM is 62 (Altschul et al., 1997, Nucleic Acids Res., 25:3389-3402).

“Sequence homology” refers to the similarity (% positives) of two polypeptide sequences determined by means of for example BLASTP 2.0.1 wherein the Filter is set off and BLOSUM is 62 (Altschul et al., 1997, Nucleic Acids Res., 25:3389-3402).

The term “liver disorder” refers to and comprises all kinds of disorders that preferably affect the anatomy, physiology, metabolic, and/or genetic activities of the liver, that preferably affect the generation of new liver cells, and/or the regeneration of the liver, as a whole or parts thereof preferably transiently, temporarily, chronically or permanently in a pathological way. Preferably also included are inherited liver disorders and neoplastic liver disorders. Liver disorder is further understood to preferably comprise liver disorders caused by trauma, intoxication, in particular by alcohol, drugs or food intoxication, radiation, infection, cholestasis, immune reactions, and by inherited metabolic liver diseases. Preferred examples of liver disorders include cirrhosis, alcoholic liver disease, chronic hepatitis, Wilson's Disease, and heamochromatosis. Preferably further included are autoimmune-disorders wherein the autoimmune response is directed against at least one polypeptide according to the invention. Within the meaning of the present invention the term “liver disorder” preferably also encompasses liver cancer, for example hepatocellular carcinoma (HCC), benign liver neoplasms such as adenoma and/or FNH. Preferably HCC further comprises subtypes of the mentioned disorders, preferably including liver cancers characterized by intracellular proteinaceous inclusion bodies, HCCs characterized by hepatocyte steatosis, and fibrolamellar HCC. For example, precancerous lesions are preferably also included such as those characterized by increased hepatocyte cell size (the “large cell” change), and those characterized by decreased hepatocyte cell size (the “small cell” change) as well as macro regenerative (hyperplastic) nodules (Anthony, P. in MacSween et al, eds. Pathology of the Liver. 2001, Churchill Livingstone, Edinburgh).

The term “epithelial cancer” within the meaning of the invention includes adenocarcinomas of any organ other than the liver, preferably of the lung, stomach, kidney, colon, prostate, skin and breast, and refers to disorders of these organs in which epithelial cell components of the tissue are transformed resulting in a malignant tumor identified according to the standard diagnostic procedures as generally known to a person skilled in the art.

Within the meaning of the invention the term “disorder according to the invention” encompasses epithelial cancer and liver disorders as defined above.

In the case of polypeptides, the term “differential expression of a polypeptide” refers to the relative level of expression of the polypeptide in an isolated sample from a patient compared to the expression of the polypeptide in a reference sample or a reference library. The expression can be determined by methods generally known to the person skilled in the art. Examples of such methods include immunohistochemical or immunoblot or ELISA detection of the polypeptide with antibodies specific for the polypeptide. Detection of the polypeptide through genetic manipulation to label the polypeptide and detection in a model system is preferably also included such as by tagging the polypeptide in a transgene for expression in a model system.

The term “sample” refers to a biomaterial comprising liver tissue or liver cells, preferably tissue from another organ subject to malignant transformation or a cell from this organ, blood, serum, plasma, ascitic fluid, pleural effusions, cerebral spinal fluid, saliva, urine, semen or feces.

The sample can be isolated from a patient or another subject by means of methods including invasive or non-invasive methods. Invasive methods are generally known to the skilled artisan and comprise for example isolation of the sample by means of puncturing, surgical removal of the sample from the opened body or by means of endoscopic instruments. Minimally invasive and non-invasive methods are also known to the person skilled in the art and include for example, collecting body fluids such as blood, serum, plasma, ascitic, pleural and cerebral spinal fluid, saliva, urine, semen, and feces. Preferably the non-invasive methods do not require penetrating or opening the body of a patient or subject through openings other than the body openings naturally present such as the mouth, ear, nose, rectum, urethra, and open wounds.

The term “minimally invasive” procedure refers to methods generally known, especially by persons skilled in the art, for obtaining patient sample material that do preferably not require anesthesia, can be routinely accomplished in a physician office or clinic and are either not painful or only nominally painful. The most common example of a minimally invasive procedure is venupuncture.

The term “reference sample” refers to a sample that serves as an appropriate control to evaluate the differential expression of a nucleic acid and/or a polypeptide according to the invention in a given sample isolated from a patient; the choice of such appropriate reference sample is generally known to the person skilled in the art. Examples of reference samples include samples isolated from a non-diseased organ or tissue or cell(s) of the same patient or from another subject, wherein the non-diseased organ or tissue or cell(s) is selected from the group consisting of liver tissue or liver cells, blood, or the samples described above. For comparison to expression in the sample isolated from a patient with the liver disorder, the reference sample may also include a sample isolated from a non-diseased organ or tissue or cell(s) of a different patient, wherein the liver disordered-tissue or cell(s) is selected from the sample group listed above. Moreover the reference may include samples from healthy donors, preferably matched to the age and sex of the patient.

The term “reference library” refers to a library of clones representing expressed genes, which library is preferably prepared from non-diseased liver tissue or cells. The reference may also derive from mRNA from non-diseased liver tissue or cells and may also comprise a data base comprising data on non-diseased tissue expression of nucleic acids. For comparison of the expression of the nucleic acids or polypeptides according to the invention in a sample isolated from a patient with the disordered liver, the reference library may comprise an expression library prepared from liver disorder-diseased liver tissue or cells and a data base comprising data on liver disorder-specific expression of nucleic acids.

The term “patient” within the meaning of the invention includes animals, preferably mammals, and humans, dead or alive. The patient is either suffering from a liver disorder, and/or other epithelial cancer, subject to analysis, preventive measures, therapy and/or diagnosis in the context of liver disorder and/or other epithelial cancer.

The term “subject” within the meaning of the invention includes animals, preferably mammals, and humans, dead or alive that are not suffering from a liver disorders and/or other epithelial cancer and thus represent a preferred appropriate control for the determination of differential expression of nucleic acids and/or polypeptides according to the invention in a patient.

The term “effective treatment” within the meaning of the invention refers to a treatment that preferably cures the patient from at least one disorder according to the invention and/or that improves the pathological condition of the patient with respect to at least one symptom associated with the disorder, preferably 3 symptoms, more preferably 5 symptoms, most preferably 10 symptoms associated with the disorder; preferably on a transient, short-term (in the order of hours to days), long-term (in the order of weeks, months or years) or permanent basis, wherein the improvement of the pathological condition may be preferably constant, increasing, decreasing, continuously changing or oscillatory in magnitude as long as the overall effect is a significant improvement of the symptoms compared with a control patient. Therapeutic efficacy and toxicity, e.g. ED₅₀ and LD₅₀ may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions that exhibit large therapeutic indexes are preferred. The dose must be adjusted to the age, weight and condition of the individual patient to be treated, as well as the route of administration, dosage form and regimen, and the result desired, and the exact dosage should of course be determined by the practitioner.

The actual dosage depends on the nature and severity of the disorder being treated, and is within the discretion of the physician, and may be varied by titration of the dosage to the particular circumstances of this invention to produce the desired therapeutic effect. However, it is presently contemplated, that pharmaceutical compositions comprising of from about 0.1 to 500 mg of the active ingredient per individual dose, preferably of from about 1 to 100 mg, most preferred from about 1 to 10 mg, are suitable for therapeutic treatments.

The active ingredient may be administered in one or several dosages per day. A satisfactory result can, in certain instances, be obtained at a dosage as low as 0.1 μg/kg intravenously (i.v.) and 1 μg perorally (p.o.). Preferred ranges are from 0.1 μg/kg/day to about 10 mg/kg/day i.v. and from 1 μg/kg/day to about 100 mg/kg/day p.o.

In another aspect the invention relates to a fusion protein comprising a polypeptide according to the SEQ ID 1 to 9 and/or SEQ ID 47, or a functional variant thereof.

A “fusion protein” refers to a polypeptide comprising at least one polypeptide according to the SEQ ID 1 to 9 and/or SEQ ID 47, a functional variant or part thereof and at least one component A selected from polypeptide, peptide and/or peptide analogue that is linked to the polypeptide according to the invention by means of covalent or non-covalent binding such as e.g. hydrogen bonds, generally known to the person skilled in the art. Preferred examples of component A for fusion proteins are polypeptide, peptide and/or peptide analogues that facilitate easier detection of the fusion proteins; these are, for example, “green-fluorescent-protein”, or variants thereof. Also included are fusion proteins that facilitate purification of the recombinant protein such as “his-tags”, or fusions that increase the immunogenicity of the protein.

Fusion proteins according to the invention can be produced by methods generally known to the person skilled in the art. The fusion proteins according to the invention can be used for the diagnosis, prevention and or treatment of liver disorders and/or epithelial cancer.

Compared to the state of the art, these fusion proteins surprisingly allow improved, more sensitive, earlier, faster, and/or non-invasive diagnosis and/or improved, sustained and/or more effective treatment of the liver disorders and/or epithelial cancers.

Preferred nucleic acids according to the invention have a sequence according to one of SEQ ID 10 to SEC ID No. 19, or a variant thereof. In particular the invention relates to nucleic acids according to the invention that have been isolated.

Compared to the state of the art, these nucleic acids and polypeptides surprisingly allow improved, more sensitive, earlier, faster, and/or non-invasive diagnosis and/or improved, sustained and/or more effective treatment of the liver disorders and/or epithelial cancers.

The term “nucleic acid according to the invention” refers to the nucleic acids corresponding to the SEQ ID 10 to SEQ ID 19 and/or variants thereof.

The term “encoding nucleic acid” relates to a DNA sequence that codes for an isolatable bioactive polypeptide according to the invention or a precursor thereof. The polypeptide can be encoded by a sequence of full length or any part of the coding sequence as long as the biological function, such as for example receptor-activity, is essentially retained (cf. definition of functional variant).

It is known that small alterations in the sequence of the nucleic acids described above can be present, for example, due to the degeneration of the genetic code, or that untranslated sequences can be attached to the 5′ and/or 3′ end of the nucleic acid without significantly affecting the activity of the encoded polypeptide. This invention, therefore, also comprises so-called naturally occurring and artificially generated “variants” of the nucleic acids described above.

Preferably, the nucleic acids used according to the invention are DNA or RNA, preferably a DNA, in particular a double-stranded DNA. In particular the nucleic acid according to the invention may be an RNA molecule, preferably single-stranded or a double-stranded RNA molecule. The sequence of the nucleic acids may further comprise at least one intron and/or one polyA sequence.

Nucleic acids according to the invention can be produced by methods generally known to the skilled artisan and have also been described in detail below.

“Variant” within the meaning of the invention refers to all DNA sequences that are complementary to a DNA sequence, which hybridize with the reference sequence under stringent conditions and have a similar activity to the corresponding polypeptide according to the invention. The nucleic acids according to the invention can also be used in the form of their antisense sequence.

“Variant” of the nucleic acids can also be homologues from other species with sequence identity preferably 80%, in particular 90%, most prefered 95%.

“Variant” of the nucleic acids can also be parts of the nucleic acid according to the present invention with at least about 8 nucleotides length, preferably with at least about 16 nucleotides length, in particular with at least about 21 nucleotides length, more preferably with at least about 30 nucleotides length, even more preferably with at least about 40 nucleotides length, most preferably with at least about 50 nucleotides length as long as the parts have a similar activity to the corresponding polypeptide according to the invention. Such activity can be assayed using the functional assays described further above.

In a preferred embodiment of the invention the nucleic acid comprises a nucleic acid having a sequence complementary to a nucleic acid according to the invention, or a variant thereof. Preferably the nucleic acid comprises a non-functional mutant variant of the nucleic acid according to the invention, or a variant thereof.

In particular the invention relates to a nucleic acid having a complementary sequence wherein the nucleic acid is an antisense molecule or an RNA interference molecule.

The term “non-functional mutant variant of a nucleic acid” refers to a nucleic acid derived from a nucleic acid according to the invention, or a variant thereof having been mutated such that the polypeptide encoded by the non-functional mutant variant of the nucleic acid exhibits a biological activity which in comparison the non-mutated polypeptide is significantly decreased or abolished. Such activity of the polypeptide encoded by the non-functional mutant variant nucleic acid can be determined by means of a functional assay as described above for the evaluation of functional variants. The construction and screening of such non-functional mutant variant derived from a nucleic acid according to the invention are generally known to the person skilled in the art. Such “non-functional mutant variant of a nucleic acid” according to the invention can be expressed in a patient and will preferably abolish or diminish the level of expression of the targeted nucleic acid by competing with the native mRNA molecules for translation into polypeptides by the ribosomes.

“Stringent hybridization conditions” refer to those conditions in which hybridization takes place at 60° C. in 2.5×SSC buffer and remains stable following a number of washing steps at 37° C. in a buffer of lower salt concentration.

The term “differential expression of a nucleic acid” refers to the relative level of expression of the nucleic acid in an isolated sample from a patient compared to the expression of the nucleic acid in a reference sample or a reference library. Definitions of reference samples and reference libraries have been described in detail above. The expression can be determined by methods generally known to the person skilled in the art. Examples of such methods include RNA blot (northern) analysis, nuclease protection, in situ hybridization, reverse transcriptase PCR (RT-PCR; including quantitative kinetic RT-PCR). cDNA and oligonucleotide microarrays are also included as such methods.

In a preferred embodiment the nucleic according to the invention is the OBcl1 cDNA (SEQ ID 10), which is assembled by identification of overlapping sequences from the non-redundant and human EST GenBank sequence databases. The expression in HCC of RNA corresponding to assembled sequence SEQ ID is confirmed experimentally. The initial sequence upregulated in HCC relative to non-diseased liver identified as an SSH cDNA clone corresponds to GenBank sequence AL050205. The 5′ end of that sequence overlaps with AF131755; this sequence is extended progressively 5′ with XM113703, AK055521 and AY004310. The latter three sequences include the open reading frame encoding OBcl1.pr (SEQ ID 1). In support of this mRNA construct, all overlapping cDNA sequences can be localized to the same chromosome. Furthermore, an mRNA of approximately 6 kilobases was identified by RNA blot analysis of HCC but not normal liver RNA using the SSH sequence from this clone as a hybridization probe (FIG. 4). Expression of sequences corresponding to this clone has not previously been reported in liver or in HCC.

In a preferred embodiment the polypeptide according to the invention is the OBcl1.pr polypeptide (SEQ ID 1) which is surprisingly identified from an mRNA identified to be upregulated in HCC by an average of 2.9-fold relative to non-diseased liver (OBcl1, SEQ ID 10) (see Table 3A/3B). cDNA sequences encoding this polypeptide and overlapping with this mRNA are identified with reverse transcriptase PCR analysis and these nucleic acids are similarly elevated in HCC. This polypeptide sequence was previously unrecognized with respect to elevated levels in HCC. From the sequence of the OBcl1.pr polypeptide, two conserved sequence domains can be identified with the conserved domain prediction CDD algorithm available with the BLAST sequence analysis tools (Altschul et al., 1997, Nucleic Acids Res., 25:3389-3402); a lupus La polypeptide type RNA binding domain (SEQ ID 1, amino acids 47 to 125), and a GTPase enzymatic domain with unknown function (SEQ ID 1, amino acids 90 to 203). The OBcl1.pr sequence has been designated in the GenBank sequence database as the cellular myeloproliferative leukemia receptor (c-Mpl) binding polypeptide. Although a potential modulator of the myeloproliferative leukemia virus receptor (also known as the thrombopoietin receptor), the functional role for this polypeptide has not been described in any system. Similarly, the expression pattern of this polypeptide has not been disclosed. The mRNA encoding this polypeptide is elevated more than 2-fold relative to non-diseased liver in 11 of 21 liver tumors subjected to expression profiling (52%). The mRNA encoding this polypeptide is similarly elevated at least 2-fold in 4 of 4 focal nodular hyperplasia (FNHs) profiled (100%) (Table 3A/3B). For this and the other nucleic acids according to the invention, this value for expression includes the expression value ratio data from all of the 21 HCC samples subjected to the cDNA microarray expression profiling experiments, including the values from samples that are not elevated by 2-fold or greater.

The expression of this mRNA is remarkably specific to liver disorders since expression is not detected in other carcinomas analyzed nor in non-diseased tissues including liver, kidney, stomach, lung, skin and others (see Table 6). Independent RT-PCR analysis of expression levels of Obcl1 mRNA are determined with gene specific oligonucleotide primers including SEQ ID 22 and SEQ ID 23. Therefore it is surprisingly found that there is a strong and specific correlation between the expression of OBcl1.pr polypeptide (SEQ ID 1) and the nucleic acid encoding the polypeptide (SEQ ID 10) respectively and the disorders according to the invention. Therefore the polypeptide and the encoding nucleic acid can be utilized for diagnosis of disorders according to the invention, for example for the diagnostic discrimination between hyperplastic (including neoplastic) liver diseases and cirrhosis.

Furthermore, expression of this HCC-deregulated gene correlates with proliferation of hepatoma cells, showing 3.4-fold and 6.3-fold increase of Obcl1 mRNA in Hep3B cell line upon 8 hours and 12 hours serum stimulation of quiescent cells, respectively (see FIG. 8).

These results demonstrate that OBcl1.pr polypeptide (SEQ ID 1) and the nucleic acid encoding the polypeptide (SEQ ID 10) can be employed in the prevention and therapy of disorders according to the invention, in particular for the treatment of hyperplastic (including neoplastic) liver diseases. With regard to the treatment it is preferred to carry out the treatment such that the expression of the OBcl1.pr polypeptide or of the nucleic acid encoding the polypeptide is reduced and/or inhibited, for example by administering antisense oligonucleotides or RNA interference molecules that specifically interact with the nucleic acid encoding the OBcl1.pr polypeptide. Alternatively the treatment may be carried out such that the activity of the OBcl1.pr polypeptide is reduced and/or inhibited, for example by administering an antibody directed against the OBcl1.pr polypeptide or an antibody fragment thereof which block the activity of the OBcl1.pr polypeptide to a patient in need of such treatment. Compared to the state of the art, this OBcl1.pr polypeptide and/or OBcl1 nucleic acid surprisingly allow improved, more sensitive, earlier, faster, and/or non-invasive diagnosis and/or improved, sustained and/or more effective treatment of the liver disorders and/or epithelial cancers.

In another preferred embodiment the nucleic acid according to the invention is the OBcl5 nucleic acid (SEQ ID 11) that is the compiled sequence encoding OBcl5.pr polypeptide (SEQ ID 2).

The entire sequence is established from a number of GenBank expressed sequence tag (EST) database sequences and GenBank genomic database sequences, and each segment is verified for overexpression in HCC. For example, the sequence for this nucleic acid on a cDNA microarray is elevated an average of 24.7-fold relative to non-diseased liver reference (Table 3A/3B).

Expression of partial sequences corresponding to this clone has been reported in several tissues and some tumors (including fetal liver, colon adenocarcinoma and in tumor metastases localized in the liver) but the entire sequence according to the invention has not previously been described. Elevated expression of OBcl5 is therefore quite specific to liver disorders. Neither OBcl5 nucleic acid nor the compiled sequence of a deduced polypeptide have been recognized with respect to elevated levels in disorders according to the invention, preferably in HCC.

Information concerning expression of this and all sequences according to the invention is obtained from searching of public domain databases (such as the PubMed and SOURCE). Journal articles have not been published for most of the sequences according to the invention. The relative abundance of cDNA clones from automatically sequenced cDNA libraries therefore provides the evidence cited herein for expression of this and other sequences according to the invention. This information is accessed via databases such as ‘SOURCE’ (provided by the Genetics Department, Stanford University) that includes data curated from UniGene, Swiss-Prot, GeneMap99, RHdb, dbEST, GeneCards and Locus-Link databases.

In another preferred embodiment the polypeptide according to the invention is the OBcl5.pr polypeptide (SEQ ID 2), which represents the largest open reading frame from this deregulated mRNA sequence. This polypeptide sequence does not contain recognized sequence homologies to characterized polypeptides or to known structural motifs. No pattern of expression has been described for this polypeptide. Expression of the RNA potentially encoding this polypeptide is elevated greater than 2 fold in 100% of HCC cases examined relative to non-diseased liver and greater than 8-fold in 17 of the 21 cases profiled (81%). Elevated expression of the encoding mRNA relative to non-diseased liver is also evident in liver adenoma, FNH, and cirrhotic livers but the transcript is less dramatically upregulated in cirrhosis. The mRNA encoding this polypeptide is detectable in non-diseased human lung, brain (cortex), colon, testis tissue but not in most other carcinomas evaluated (Table 6.). Independent RT-PCR analyses of expression levels of Obcl5 RNA are determined with gene specific oligonucleotide primers including SEQ ID 24 and SEQ ID 25. High expression specificity of the OBcl5 cDNA is confirmed by quantitative assessment (Q-PCR) in HCC, FNH in comparison to expression pattern in normal tissue(s) and other types of cancer as illustrated in FIG. 2. The TaqMan procedure utilizing the parallel examination of both GAPDH and β-actin as reference genes confirms a large over expression of OBcl5 RNA (SEQ ID 11) in HCC and FNH compared with non-neoplastic liver (FIG. 2). Relative to these housekeeping genes, Q-PCR reveals that OBcl5 RNA levels are elevated in liver cancer and FNH compared with normal liver and that OBcl5 RNA levels are much lower in other tissues and in other cancers than in the normal liver. For TaqMan analyses OBcl5 expression was determined with gene specific oligonucleotide primers including SEQ ID 66; SEQ ID 67 and SEQ ID 68 (the ‘hydrolysis’ probe).

Furthermore, in situ hybridization analyses clearly indicate localization of OBcl5 RNA in HCC in contrast to marginal signal in normal liver tissue sections by employing a radioisotope labeled OBcl5 RNA antisense probe that specifically hybridises with OBcl5 RNA (FIG. 5).

Overexpression of the polypeptide and/or the encoding RNA therefore, may be useful for diagnosis of liver disorders. These results clearly demonstrate that the OBcl5.pr polypeptide and the nucleic acid encoding the polypeptide (SEQ ID 11) and a functional variant thereof can be utilized for diagnosis, prevention and treatment of disorders according to the invention, in particular for HCC, liver adenoma, FNH and cirrhosis.

With regard to the treatment it is preferred to carry out the treatment such that the expression of the OBcl5.pr polypeptide and/or a functional variant thereof; or of the nucleic acid encoding the polypeptide and/or a functional variant thereof is reduced and/or inhibited, for example by administering antisense oligonucleotides or small interfering RNA molecules that specifically interact with the nucleic acid defined in SEQ ID 11 potentially encoding the OBcl5.pr polypeptide and/or a functional variant thereof.

Alternatively the treatment may be carried out such that the activity of the OBcl5.pr polypeptide and/or a functional variant thereof are reduced and/or inhibited, for example by administering an antibody directed against the OBcl5.pr polypeptide and/or a functional variant thereof, or an antibody fragment thereof which block the activity of the OBcl5.pr polypeptide and/or a functional variant thereof to a patient in need of such treatment. Compared to the state of the art, the OBcl5.pr polypeptide and/or a functional variant thereof; and/or OBcl5 nucleic acid surprisingly allow improved, more sensitive, earlier, faster, and/or non-invasive diagnosis and/or improved, sustained and/or more effective treatment of the liver disorders and/or other epithelial cancers.

Detailed sequence analysis revealed sequence similarities between OBcl5 mRNA to other eukaryotic non-coding RNAs. In addition, multiple attempts with diverse methodologies to detect a protein product from this RNA have not revealed such a product. Therefore, this RNA may be not translated into a polypeptide but may have functional (e.g., regulatory) properties itself. The disease relevance of non-coding regulatory RNAs is now becoming apparent as evidenced, for example, by the role of the non-coding RNA “bantam” involved in cellular proliferation in the eukaryote Drosophila (Brennecke J, Hipfner D R, Stark A, Russell R B, Cohen S M. Cell (2003) April 4; 113(1):25-36), and by microRNA-23 that interacts with the transcription factor HES-1 to hinder neuronal differentiation (Kawasaki, H. and Tiara, K. Nature (2003) 423:838-842).

Reduction of the level of OBcl5 RNA (knock-down) in proliferating human hepatoma cells with small interfering RNA (siRNA) oligonucleotides supports a functionally significant role for elevated expression of OBcl5 RNA in liver disorders, especially liver cancer. In this experiment, the level of mRNA encoding the tumor suppressor gene retinoblastoma protein 1 (RB1) is upregulated several-fold upon decreasing the level of OBcl5 RNA, determined with TaqMan Q-PCR as described above. RB1 mRNA levels are determined with SYBR Green quantitative PCR analyses using primers RB1-p1 (SEQ ID 64) and RB1-p2 (SEQ ID 65). By a negative regulation of the RB1, elevated expression of OBcl5 RNA in HCC may therefore facilitate tumor cell growth (FIG. 6).

In a yet another preferred embodiment the nucleic acid according to the invention is the IK2 nucleic acid (SEQ ID 12) represented by the Gene Bank sequence NM_{—}025160 which includes the open reading frame encoding IK2.pr polypeptide (SEQ ID 3). The IK2.pr polypeptide is another embodiment of the invention. EST sequences corresponding to this clone have been reported in cDNA libraries from several tissues including liver and in adenocarcinomas, but the sequence has not previously been implicated in HCC. Expression of this polypeptide has not been described in any cell or tissue. The polypeptide sequence has no known function although the sequence is evolutionarily well conserved (predicted polypeptides are found in several mammals, fruit fly (Drosophila) and plants (Arabidopsis). The CDD algorithm predicts several WD40-type polypeptide-polypeptide interaction domains in this polypeptide sequence according to the invention. In liver samples from HCC patients expression of the mRNA encoding this polypeptide is surprisingly elevated relative to non-diseased liver by an average value of 4.67-fold in 15 of the 21 cases profiled (71%). Elevated expression of the encoding mRNA relative to non-diseased liver is also evident in cirrhotic livers (Table 3A/3B). Highest differential expression levels of the mRNA encoding this peptide relative to non-diseased liver are observed in FNH; 8-fold upregulation in 4 of 4 cases profiled. The mRNA encoding this polypeptide is also expressed in several other human carcinomas including those of the mammary gland, lung and kidney, and in 2 (breast and kidney) of the 17 non-diseased human tissues examined. Independent RT-PCR analysis of expression levels of IK2 mRNA were determined with gene specific oligonucleotide primers including SEQ ID 26 and SEQ ID 27.

These results demonstrate that the overexpression of this polypeptide and/or the encoding mRNA, can be utilized for the diagnosis, prevention and treatment of disorders according to the invention, in particular for the diagnosis of HCC, FNH, cirrhosis, and epithelia-derived neoplasms. With regard to the treatment it is preferred to carry out the treatment such that the expression of the IK2.pr polypeptide or of the nucleic acid encoding the polypeptide is reduced and/or inhibited, for example by administering antisense oligonucleotides or RNA interference molecules that specifically interact with the nucleic acid encoding the IK2.pr polypeptide. Alternatively the treatment may be carried out such that the activity of the IK2.pr polypeptide is reduced and/or inhibited, for example by administering an antibody directed against the IK2.pr polypeptide or an antibody fragment thereof which block the activity of the IK2.pr polypeptide to a patient in need of such treatment. Compared to the state of the art, this IK2.pr polypeptides and/or IK2 nucleic acid surprisingly allow improved, more sensitive, earlier, faster, and/or non-invasive diagnosis and/or improved, sustained and/or more effective treatment of the liver disorders and/or other epithelial cancers.

In a yet another preferred embodiment the nucleic acid according to the invention is the IK5 nucleic acid (SEQ ID 13) that represents the sequence of an HCC deregulated cDNA clone. Expression of sequences corresponding to this clone has been reported in several tissues (including liver) and some tumors (including pituitary and prostate) but the sequence has not previously been described to be upregulated in HCC. In a preferred embodiment the polypeptide according to the invention is the IK5.pr polypeptide (SEQ ID 4) that is encoded by the IK5 cDNA (SEQ ID 13). The polypeptide sequence is deduced from the GenBank database (Accession number: NM_{—}006407) as JWA, a vitamin A responsive polypeptide. Although the gene encoding this putative polypeptide has been described from stimulation of cultured cells with vitamin A, the presence of the polypeptide has not been described in any cell or tissue and the function is unknown. JWA is further described as a cytoskeleton-associated polypeptide in the GenBank database. The polypeptide shares homology also with rodent polypeptides that interact specifically with and may reduce the activity of the EAAC1 glutamate transporter. A conserved domain search of this sequence indicates the likely presence of a prenylated rab acceptor 1 domain (PRA1), possibly mediating interaction with G protein signaling molecules. Expression of the mRNA encoding this polypeptide is elevated by an average of 9.14-fold relative to non-diseased liver in 100% of the HCC cases profiled. Similarly, elevated expression of the encoding mRNA is also evident in Adenoma and FNH. The encoding mRNA expression is differentially expressed also in cirrhotic livers but to a lesser extent than in the other liver disorders. The mRNA encoding this polypeptide is expressed in lung, kidney and colon human carcinomas but in just 1 of the 17 non-diseased human tissues examined. Independent RT-PCR analyses of expression levels of IK5 mRNA are determined with gene specific oligonucleotide primers including SEQ ID 28 and SEQ ID 29. Overexpression of this polypeptide and/or the encoding mRNA may mark specific epithelia-derived neoplasms, including liver cancer. These results show that the differential upregulated expression of the IK5 cDNA sequence is highly specific for disorders according to the invention.

Furthermore, the expression of this HCC-deregulated gene correlates with proliferation of hepatoma cells, showing 10.9-fold and 4.3-fold increase of IK5 mRNA in Hep3B cell line upon 8 hours and 12 hours serum stimulation of quiescent cells, respectively (see FIG. 8).

Therefore the IK5.pr polypeptide and/or the encoding nucleic acid can be utilized for the diagnosis, prevention and treatment of disorders according to the invention, in particular for the diagnosis of HCC, adenoma, FNH, cirrhosis, and epithelia-derived neoplasms. With regard to the treatment it is preferred to carry out the treatment such that the expression of the IK5.pr polypeptide or of the nucleic acid encoding the polypeptide is reduced and/or inhibited, for example by administering antisense oligonucleotides or RNA interference molecules that specifically interact with the nucleic acid encoding the IK5.pr polypeptide. Alternatively the treatment may be carried out such that the activity of the IK5.pr polypeptide is reduced and/or inhibited, for example by administering an antibody directed against the IK5.pr polypeptide or an antibody fragment thereof which block the activity of the IK5.pr polypeptide to a patient in need of such treatment. Compared to the state of the art, this IK5.pr polypeptide and/or IK5 nucleic acid surprisingly allow improved, more sensitive, earlier, faster, and/or non-invasive diagnosis and/or improved, sustained and/or more effective treatment of the liver disorders and/or other epithelial cancers.

In yet another preferred embodiment the nucleic acid according to the invention is the DAP3 nucleic acid (SEQ ID 14) which has been disclosed before (Accession. No. X83544) encoding the DAP3.pr polypeptide (SEQ ID 5). The invention further relates to the death associated polypeptide 3 (DAP3, SEQ ID 5) which has been implicated in promotion of apoptotic cell death when overexpressed in cultured cells (Kissil et al., 1995, J. Biol. Chem., 270:27932-6).

The polypeptide contributes to the mitochondrial 28S ribosomal complex. As such, this polypeptide is likely to be ubiquitously expressed in many if not all tissues and cells, albeit apparently at relatively low levels. No specific function for endogenous DAP3 has been described (Cadvar Koc et al., 2001, FEBS Lett., 492:166-170). Down-regulation of DAP3 mRNA is described in colon adenocarcinoma metastates in liver (PCT/US01/30589), but neither DAP3 nucleic acid nor the DAP3 polypeptide have been recognized with respect to elevated levels in disorders according to the invention, preferably in HCC.

Quantitative RT PCT (Q-PCR) amplification analysis of purified genomic DNA suggests DAP3 gene amplification in liver cancer with approximately 4-6 copies in 8 of 10 HCC cases and no amplification in 13 from 13 non-neoplastic liver samples (including tumor proximal and distal cirrhotic tissues). These analyses are performed with the TaqMan procedure to precisely quantify the relative amount of DAP3 genomic DNA using primers DAP3-p5 (SEQ ID 71), DAP3-p6 (SEQ ID 72) and the hydrolysis probe DAP3 p-7 (SEQ ID 73). Indeed, the DAP3 gene is located on chromosome 1q, a region frequently found to be amplified in HCC (Buendia Mass., Med Pediatr Oncol. (2002) November; 39(5):530-5.) This finding suggests that a positive selective force manifested as gene amplification may drive the over-expression of DAP3 RNA in HCC, supporting a functionally significant role for DAP3 in HCC.

Expression of the mRNA encoding this polypeptide is elevated an average of 5.5-fold relative to non-diseased liver in 18 of the 21 HCC cases profiled (86%). Elevated expression of the encoding mRNA is also evident in other liver disorders but to a lesser extent than in HCC. Independent RT-PCR analyses of expression levels of DAP3 mRNA are determined with gene specific oligonucleotide primers including SEQ ID 30 and SEQ ID 31. Elevated DAP3 mRNA in HCC compared with normal liver is further confirmed by Q-PCR analysis with the SYBR green technique using β-actin as a reference gene. In RNA isolated from each of 5 HCCs examined, the DAP3 mRNA to β-actin mRNA level ratios were elevated compared to these ratios in RNA isolated from 2 normal liver samples (average HCC ratio DAP3 mRNA to β-actin mRNA=12.8; average normal liver ratio DAP3 mRNA to β-actin mRNA=1.03). Q-PCR analyses of DAP3 mRNA levels are determined with SYBR Green analyses with gene specific oligonucleotide primers including SEQ ID 69 and SEQ ID 70.

The expression of DAP3 protein is remarkably specific upregulated in HCC since expression is very low or not detected in other carcinomas analyzed nor in non-diseased tissues including liver, kidney, stomach, lung, skin and others. The functional involvement of DAP3 in HCC is further supported by this specific increase in DAP3 protein expression levels in HCC compared with normal liver and compared with other normal and diseased tissues (see Table 6 and FIG. 7). Experimental reduction of DAP3 mRNA in hepatoma cells with small interfering RNA molecules (siRNA; SEQ ID 54 and SEQ ID 55) results in dramatic morphologic and apparent biochemical changes in the hepatoma cells so that the cells enlarge and RNA and protein extraction with standard methods is not possible from treated cells. These findings further support the functional significance of increased DAP3 in HCC

These results show that the strongly upregulated expression of the DAP3 cDNA sequence and of the DAP3 pr. polypeptide are highly specific for disorders according to the invention, especially in HCC. Therefore the DAP3 polypeptide and/or the encoding nucleic acid can be utilized for the diagnosis, prevention and treatment of disorders according to the invention, in particular for the diagnosis of HCC. With regard to the treatment it is preferred to carry out the treatment such that the expression of the DAP3 polypeptide or of the nucleic acid encoding the polypeptide is reduced and/or inhibited, for example by administering antisense oligonucleotides or RNA interference molecules that specifically interact with the nucleic acid encoding the DAP3 polypeptide. Alternatively the treatment may be carried out such that the activity of the DAP3 polypeptide is reduced and/or inhibited, for example by administering an antibody directed against the DAP3 polypeptide or an antibody fragment thereof which block the activity of the DAP3 polypeptide to a patient in need of such treatment. Compared to the state of the art, this DAP3 polypeptide and DAP3 nucleic acid surprisingly allow improved, more sensitive, earlier, faster, and/or non-invasive diagnosis and/or improved, sustained and/or more effective treatment of the liver disorders and/or other epithelial cancers.

In another preferred embodiment invention relates to the HCC up-regulated LOC5.pr hypothetical polypeptide (SEQ ID 6) and to the nucleic acid LOC5 (SEQ ID 15) coding for the polypeptide. cDNA corresponding to this mRNA has been identified in cDNA libraries from several human tissues including liver (information from SOURCE database as described above) but the sequence has not previously been reported to be up-regulated in disorders according to the invention, in particular in HCC. Expression of this mRNA is elevated 5-fold relative to non-diseased liver in 71% of the HCC cases profiled (Table 3B). Similar analysis reveals elevated expression of this mRNA in FNH and in a majority of cirrhotic livers subjected to this cDNA microarray expression profiling procedure. The mRNA is expressed in other human gastrointestinal tract carcinomas but only in brain and bone marrow of the 17 non-diseased human tissues examined. Independent RT-PCR analyses of expression levels of LOC5 mRNA are determined with gene specific oligonucleotide primers including SEQ ID 32 and SEQ ID 33. LOC5.pr (SEQ ID 6) is a predicted 30 kDa polypeptide (Accession number NP_{—}060917.1 in the GenBank database). The presence of this polypeptide has not been described in any cell or tissue. No function has been described for this predicted polypeptide and no conserved domains are revealed from a search with the CDD domain algorithm. These results show that the strongly upregulated expression of the LOC5 cDNA sequence is highly specific for disorders according to the invention, especially in HCC, FNH and in a majority of cirrhotic livers. Furthermore, expression of this HCC-deregulated gene correlates with proliferation of hepatoma cells, showing 3.7-fold and 8.8-fold fold increase of LOC5 mRNA in Hep3B cell line upon 8 hours and 12 hours serum stimulation of quiescent cells, respectively (see FIG. 8).

Therefore the LOC5.pr polypeptide and/or a functional variant thereof and/or the encoding nucleic acid and/or a variant thereof can be utilized for the diagnosis, prevention and treatment of disorders according to the invention, in particular for the diagnosis of in HCC, FNH, and a majority of cirrhotic livers. With regard to the treatment it is preferred to carry out the treatment such that the expression of the LOC5.pr polypeptide or of the nucleic acid encoding the polypeptide is reduced and/or inhibited, for example by administering antisense oligonucleotides or RNA interference molecules that specifically interact with the nucleic acid encoding the LOC5.pr polypeptide. Alternatively the treatment may be carried out such that the activity of the LOC5.pr polypeptide is reduced and/or inhibited, for example by administering an antibody directed against the LOC5.pr polypeptide or an antibody fragment thereof which block the activity of the LOC5.pr polypeptide to a patient in need of such treatment. Compared to the state of the art, this LOC5.pr polypeptide and/or LOC5 nucleic acid surprisingly allow improved, more sensitive, earlier, faster, and/or non-invasive diagnosis and/or improved, sustained and/or more effective treatment of the liver disorders and/or other epithelial cancers.

In a further preferred embodiment the invention relates to the SEC14L2 nucleic acid cDNA (SEQ ID 16) encoding the SEC14L2.pr polypeptide (SEQ ID 7) according to the invention. The expression of SEC14L2 mRNA, has been described in many tissues but elevation of this message or the encoded polypeptide has not been previously reported in disorders according to the invention in particular not in liver disorders or cancer. SEC14L2.pr (SEQ ID 7) is a human homologue of the yeast sec polypeptide 14. Although implicated in the yeast secretory pathway, a clear function for this polypeptide or its homologues has not been described in any species. This human sequence has also been suggested to bind to tocopherol and it has been predicted that this polypeptide is involved in squalene transfer, cholesterol biosynthesis or more generally in intracellular transport (Zimmer et al., 2000, J. Biol. Chem. 275:25672-25680). Expression of this polypeptide sequence has not been reported in human cells or tissues. The polypeptide sequence includes possible G-polypeptide binding and phosphotidylinositol transfer domains and a consensus CRAL_TRIO domain. The latter has been implicated in vitamin binding via the cis-retinal CRAL motif. The mRNA encoding this polypeptide is elevated an average of 5.14-fold or greater relative to non-diseased liver in 71% of HCC samples, in all FNH disease samples profiled, but not in adenoma in only one-half of cirrhosis samples (Table 3A/3B). Expression of the mRNA encoding this polypeptide has been detected in kidney and colon carcinoma and in the normal pancreas but not in other normal tissues examined (Table 6). Independent RT-PCR analyses of expression levels of SEC14L2 mRNA are determined with gene specific oligonucleotide primers including SEQ ID 34 and SEQ ID 35. Furthermore, expression of this HCC-deregulated gene correlates with proliferation of hepatoma cells, showing 10.6-fold and 1.9-fold increase of SEC14L2 mRNA in Hep3B cell line upon 8 hours and 12 hours serum stimulation of quiescent cells, respectively (see FIG. 8).

These results show that the strongly upregulated expression of the SEC14L2 cDNA sequence is highly specific for disorders according to the invention, especially in HCC and FNH. Therefore the SEC14L2.pr polypeptide and/or the encoding nucleic acid can be utilized for the diagnosis, prevention and treatment of disorders according to the invention, in particular for the diagnosis of HCC, FNH and preferably also in cirrhosis. With regard to the treatment it is preferred to carry out the treatment such that the expression of the SEC14L2.pr polypeptide or of the nucleic acid encoding the polypeptide is reduced and/or inhibited, for example by administering antisense oligonucleotides or RNA interference molecules that specifically interact with the nucleic acid encoding the SEC14L2.pr polypeptide. Alternatively the treatment may be carried out such that the activity of the SEC14L2.pr polypeptide is reduced and/or inhibited, for example by administering an antibody directed against the SEC14L2.pr polypeptide or an antibody fragment thereof which block the activity of the SEC14L2.pr polypeptide to a patient in need of such treatment. Compared to the state of the art, this SEC14L2.pr polypeptide and/or SEC14L2 nucleic acid surprisingly allow improved, more sensitive, earlier, faster, and/or non-invasive diagnosis and/or improved, sustained and/or more effective treatment of the liver disorders, and/or other epithelial cancers.

In a further preferred embodiment the invention relates to a nucleic acid (SEQ ID 17) coding for the SSP29.pr or APRIL polypeptide, which has been described in many tissues and tumors. The gene encoding this putative tumor necrosis family member has not previously been reported to be expressed at elevated levels in disorders according to the invention, in particular in HCC. Furthermore the invention relates to the silver stainable 29 kDa polypeptide (SSP29.pr; SEQ ID 8) which is encoded by the nucleic acid (SEQ ID 17) according to the invention. The polypeptide has been identified as a leucine rich secreted polypeptide, likely belonging the TNF cytokine family. It is also known as APRIL (acidic polypeptide rich in leucines) and contains leucine rich repeats (LRRs) near the N-terminus that may be involved in antigen-mediated cellular responses. (Zhu et al., 1997, Biochem. Mol. Biol. Int. 42:927-935; Mencinger et al., 1998, Biochim. Biophys. Acta 1395: 176-180). Expression of the SSP29.pr polypeptide has not been reported in human cells or tissues. The mRNA encoding this polypeptide is elevated an average of 3.77-fold relative to non-diseased liver in 17 of 21 HCCs profiled. Surprisingly, the level of the mRNA encoding this polypeptide is 30-fold higher in cirrhosis caused by copper toxicity than in a pool of non-diseased liver (Table 3A/3B). mRNA levels are marginally elevated in other liver disorders profiled relative to non-diseased liver and this mRNA is otherwise detected only infrequently in the normal and diseased tissues subjected here to expression profiling. Independent RT-PCR analyses of expression levels of SSP29 mRNA are determined with gene specific oligonucleotide primers including SEQ ID 36 and SEQ ID 37. Furthermore, expression of this HCC-deregulated gene correlates with proliferation of hepatoma cells, showing 2.4-fold and 4.3-fold increase of SSP29 mRNA in Hep3B cell line upon 8 hours and 12 hours serum stimulation of quiescent cells, respectively (see FIG. 8).

These results show that the strongly upregulated expression of the SSP29 cDNA sequence is highly specific for disorders according to the invention, especially in HCC, and certain types of cirrhosis disease.

Therefore the SSP29.pr polypeptide and/or the encoding nucleic acid can be utilized for the diagnosis, prevention and treatment of disorders according to the invention, in particular for the diagnosis of HCC and cirrhosis. With regard to the treatment it is preferred to carry out the treatment such that the expression of the SSP29.pr polypeptide or of the nucleic acid encoding the polypeptide is reduced and/or inhibited, for example by administering antisense oligonucleotides or RNA interference molecules that specifically interact with the nucleic acid encoding the SSP29.pr polypeptide. Alternatively the treatment may be carried out such that the activity of the SSP29.pr polypeptide is reduced and/or inhibited, for example by administering an antibody directed against the SSP29.pr polypeptide or an antibody fragment thereof which block the activity of the SSP29.pr polypeptide to a patient in need of such treatment. Compared to the state of the art, this SSP29.pr polypeptide and/or SSP29 nucleic acid surprisingly allow improved, more sensitive, earlier, faster, and/or non-invasive diagnosis and/or improved, sustained and/or more effective treatment of the liver disorders, and/or other epithelial cancers.

In yet another preferred embodiment the invention relates to the HS16 nucleic acid (SEQ ID 18). cDNA clones corresponding to the HS16 mRNA have been identified in several tissues including adenocarcinoma of the colon but neither this mRNA nor the encoded polypeptide (HS16.pr, SEQ ID 9) have been previously implicated in disorders according to the invention, in particular in liver disorders or in HCC. The invention further relates to the polypeptide encoding for the HS 16 is a predicted polypeptide of 16.7 kDa (SEQ ID 9; Accession number NP_{—}057223 in the GenBank database). The presence of the polypeptide has not been described in any cell or tissue and its function has not been described nor are functional domains identified with the CDD algorithm. mRNA encoding this polypeptide is elevated at least 2.8-fold or higher in 8 of the HCCs examined and by nearly 2-fold in an additional 4 HCC samples examined, all relative to non-diseased liver (Table 3A/3B). Independent RT-PCR analyses of expression levels of HS16 mRNA are determined with gene specific oligonucleotide primers including SEQ ID 38 and SEQ ID 39. These results show that the strongly upregulated expression of the HS 16 cDNA sequence is highly specific for disorders according to the invention, especially in HCC.

Therefore the HS16.pr polypeptide and/or the encoding nucleic acid can be utilized for the diagnosis, prevention and treatment of disorders according to the invention, in particular for the diagnosis of HCC. With regard to the treatment it is preferred to carry out the treatment such that the expression of the HS16.pr polypeptide or of the nucleic acid encoding the polypeptide is reduced and/or inhibited, for example by administering antisense oligonucleotides or RNA interference molecules that specifically interact with the nucleic acid encoding the HS 16.pr polypeptide. Alternatively the treatment may be carried out such that the activity of the HS16.pr polypeptide is reduced and/or inhibited, for example by administering an antibody directed against the HS 16.pr polypeptide or an antibody fragment thereof which block the activity of the HS16.pr polypeptide to a patient in need of such treatment. Compared to the state of the art, this HS16.pr polypeptide and/or HS 16 nucleic acid surprisingly allow improved, more sensitive, earlier, faster, and/or non-invasive diagnosis and/or improved, sustained and/or more effective treatment of the liver disorders and/or other epithelial cancers.

In a preferred embodiment the nucleic according to the invention is the IK3 cDNA (SEQ ID 19), which was assembled by identification of overlapping sequences from the non-redundant GenBank sequence databases. The initial sequence upregulated in HCC relative to non-diseased liver identified with cDNA microarray analysis corresponds to a fetal brain cDNA in the GenBank database (AL049338). That sequence overlaps with XM_{—}131462 (SEQ ID. No. 47); a mouse cDNA encoding a protein tyrosine phosphatase receptor type D (PTPRD). Although this mouse PTPRD is highly homologous with the human PTPRD transcription unit, the region of homology with this liver cancer deregulated RNA is not found in this human PTPRD transcription unit sequence. Therefore it may be that this HCC-regulated sequence encodes a not yet described human PTPRD. Alternatively, the provided database sequence may include an error(s) that account for the lack of an open reading frame. Yet another alternative is that the encoded polypeptide may result from one of the small open reading frames in this sequence. Even further, this RNA may be not translated into polypeptide but may have functional (e.g., regulatory) properties itself.

Surprisingly the sequence from this mRNA is represented at much higher levels in HCC than in normal human liver. Otherwise this RNA is expressed at only low levels in normal brain, skeletal muscle, prostate and liver. This mRNA is elevated an average of 3.81-fold or more relative to non-diseased liver in 12 of the 21 HCC samples profiled (57%). IK3 is also elevated 2-fold or more relative to non-diseased liver in 3 of 4 FNH examined, in adenoma and in 5 of the 6 cirrhosis samples examined (Table 3A/3B). Independent RT-PCR analyses of expression levels of IK3 mRNA are determined with gene specific oligonucleotide primers including SEQ ID 40 and SEQ ID 41. These results show that the strongly upregulated expression of the IK3 cDNA sequence is highly specific for disorders according to the invention, especially in HCC, FNH, adenoma and cirrhosis.

Therefore the IK3 polypeptide and/or a functional variant thereof, and/or the encoding nucleic acid and/or a variant thereof can be utilized for the diagnosis, prevention and treatment of disorders according to the invention, in particular for the diagnosis of in HCC, FNH, adenoma and cirrhosis. With regard to the treatment it is preferred to carry out the treatment such that the expression of the polypeptide encoded by the IK3 or of the IK3 nucleic acid is reduced and/or inhibited, for example by administering antisense oligonucleotides or RNA interference molecules that specifically interact with the IK3 nucleic acid. Alternatively the treatment may be carried out such that the activity of the IK3 polypeptide is reduced and/or inhibited, for example by administering an antibody directed against the IK3 polypeptide or an antibody fragment thereof which block the activity of the IK3 polypeptide to a patient in need of such treatment. Compared to the state of the art, this IK3 nucleic acid surprisingly allows improved, more sensitive, earlier, faster, and/or non-invasive diagnosis and/or improved, sustained and/or more effective treatment of the liver disorders and/or other epithelial cancers.

The cDNA expression levels relative to a non-diseased liver reference sample of sequences according to the invention assessed in tissues from human liver disorders, including HCC are shown in Tables 3A/3B representing two independent sets of experiments. The values in Table 3B represent log2 ratios of expression levels whereas Table 3A are non-transformed data between diseased and non-diseased samples obtained from competitive hybridisation to custom-made cDNA microarrays. HCC=hepatocellular carcinoma samples; HCC (1HB)=intrahyaline body comprising HCC samples; FNH=focal nodular hyperplasia samples; Cirrh=cirrhosis samples. Mean; median (50^{th} percentile of values) and standard deviation of values for each sequence (SEQ ID 10 to 19) per group (HCC, FNH and Cirrh) are provided.

TABLE 3A
c DNA microarray expression level ratios (non-transformed values)

| | OBcl1 | OBcl5 | IK2 | IK5 | DAP3 (A) | DAP3 (B) | LOC5 | | SSP29 | HS16 | IK3 |
|---|---|---|---|---|---|---|---|---|---|---|---|
| Disease | SEQ | SEQ | SEQ | SEQ | SEQ | SEQ | SEQ | SEC14L2 | SEQ | SEQ | SEQ |
| sample | ID 10 | ID 11 | ID 12 | ID 13 | ID 14 | ID 14 | ID 15 | SEQ ID 16 | ID 17 | ID 18 | ID 19 |
| HCC11 | 4.0 | 27.7 | 2.4 | 8.8 | 2.2 | 4.7 | 1.0 | 15.6 | 2.0 | 1.1 | 1.5 |
| HCC12 | 1.5 | 38.2 | 1.1 | 11.5 | 3.1 | 6.5 | 4.3 | 14.0 | 3.8 | 1.9 | 2.1 |
| HCC13 | 1.4 | 44.6 | 7.8 | 6.7 | 7.3 | 9.3 | 7.1 | 3.3 | 2.1 | 1.8 | 2.8 |
| HCC15 | 2.6 | 30.5 | 1.4 | 3.7 | 23.9 | 3.3 | 5.8 | 3.9 | 8.6 | 1.9 | 1.7 |
| HCC1 | 2.4 | 40.5 | 7.6 | 9.6 | 1.8 | 2.4 | 7.0 | 9.4 | 5.6 | 1.6 | 12.8 |
| HCC27 | 11.6 | 11.8 | 4.2 | 2.5 | 6.2 | 2.5 | 8.2 | 4.6 | 5.0 | 9.2 | 7.1 |
| HCC29 | 10.9 | 22.9 | 13.5 | 3.9 | 6.7 | 7.6 | 5.4 | 1.9 | 7.0 | 4.7 | 3.4 |
| HCC2 | 2.2 | 41.4 | 8.3 | 5.4 | 2.5 | 9.3 | 8.9 | 2.3 | 1.9 | 1.8 | 12.5 |
| HCC30n | 1.9 | 23.8 | 0.9 | 21.0 | 2.5 | 3.4 | 3.6 | 5.5 | 10.4 | 1.7 | 0.8 |
| HCC31 | 1.3 | 9.7 | 0.6 | 13.8 | 2.9 | 3.4 | 3.0 | 0.9 | 3.3 | 3.6 | 0.9 |
| HCC32 | 2.8 | 7.8 | 4.4 | 6.0 | 3.4 | 3.1 | 3.5 | 4.4 | 3.0 | 4.7 | 3.2 |
| HCC33 | 0.9 | 7.1 | 2.0 | 4.1 | 1.9 | 3.4 | 8.3 | 7.2 | 1.9 | 0.9 | 3.4 |
| HCC34 | 2.9 | 48.3 | 9.4 | 21.7 | 3.8 | 8.5 | 12.9 | 16.3 | 1.1 | 1.4 | 7.6 |
| HCC35 | 4.0 | 3.1 | 4.2 | 7.2 | 5.4 | 2.8 | 4.3 | 3.3 | 5.0 | 4.9 | 3.0 |
| HCC36 | 1.8 | 36.3 | 5.0 | 8.4 | 5.3 | 4.6 | 6.1 | 3.3 | 2.4 | 1.4 | 1.3 |
| HCC4 | 1.7 | 21.4 | 8.3 | 15.4 | 10.6 | 19.0 | 2.0 | 0.9 | 2.5 | 3.3 | 1.8 |
| HCC6 | 0.7 | 15.6 | 0.5 | 1.9 | 1.4 | 2.3 | 1.4 | 1.6 | 2.6 | 4.3 | 1.6 |
| HCC9 | 1.2 | 52.7 | 3.6 | 15.6 | 2.7 | 2.3 | 1.1 | 4.4 | 3.8 | 1.4 | 0.9 |
| HCC (IHB) | 0.6 | 20.4 | 8.4 | 14.0 | 19.2 | 10.0 | 9.8 | 1.2 | 2.5 | 5.1 | 4.8 |
| HCC22 | 3.2 | 10.5 | 2.2 | 5.0 | 2.4 | 1.7 | 0.8 | 2.4 | 2.7 | 1.2 | 5.1 |
| HCC28 | 0.6 | 5.3 | 2.3 | 5.6 | 1.3 | 1.4 | 0.7 | 1.4 | 1.8 | 1.1 | 1.9 |
| HCC mean | 2.9 | 24.7 | 4.7 | 5.1 | 5.6 | 5.3 | 5.0 | 5.1 | 3.8 | 2.8 | 3.8 |
| HCC median | 1.9 | 22.9 | 4.2 | 7.2 | 3.1 | 3.4 | 4.3 | 3.3 | 2.7 | 1.8 | 2.8 |
| HCC std. | 3.0 | 15.4 | 3.6 | 5.8 | 5.9 | 4.2 | 3.4 | 4.8 | 2.4 | 2.1 | 3.5 |
| deviation | | | | | | | | | | | |
| FNH1 | 2.5 | 7.0 | 8.0 | 10.1 | 4.6 | 1.9 | 10.2 | 7.1 | 2.3 | 4.9 | 0.9 |
| FNH2 | 4.7 | 7.1 | 10.9 | 16.2 | 2.2 | 4.4 | 7.1 | 4.2 | 2.2 | 2.1 | 16.6 |
| FNH3 | 3.0 | 4.2 | 9.5 | 11.5 | 1.5 | 2.6 | 9.6 | 6.0 | 1.0 | 2.1 | 9.9 |
| FNH9 | 3.4 | 15.1 | 7.7 | 9.9 | 1.7 | 3.2 | 2.4 | 3.8 | 0.9 | 1.3 | 7.5 |
| FNH mean | 3.4 | 8.3 | 9.1 | 11.9 | 2.5 | 3.0 | 7.3 | 5.3 | 1.6 | 2.6 | 8.7 |
| FNH median | 3.2 | 7.1 | 8.8 | 10.8 | 2.0 | 2.9 | 8.4 | 5.1 | 1.6 | 2.1 | 8.7 |
| FNH std. | 0.9 | 4.7 | 1.5 | 2.9 | 1.4 | 1.1 | 3.5 | 1.5 | 0.8 | 1.6 | 6.5 |
| deviation | | | | | | | | | | | |
| Cirrh34b | 7.6 | 17.7 | 6.0 | 6.0 | 13.7 | 3.2 | 9.3 | 2.3 | 19.6 | 8.6 | 4.2 |
| Cirrh5 | 0.5 | 2.7 | 12.9 | 2.7 | 1.2 | 3.0 | 10.3 | 4.0 | 16.0 | 2.0 | 3.9 |
| Cirrh1 | 1.0 | 1.8 | 2.2 | 2.8 | 7.5 | 3.0 | 1.9 | 2.3 | 9.3 | 12.2 | 10.1 |
| Cirrh2 | 0.4 | 2.6 | 2.9 | 2.9 | 13.9 | 0.9 | 2.4 | 3.3 | 1.8 | 1.3 | 2.7 |
| Cirrh3 | 0.4 | 4.0 | 15.2 | 22.1 | 1.3 | 2.8 | 1.4 | 0.8 | 2.4 | 3.6 | 1.7 |
| Cirrh4 | 0.8 | 10.8 | 24.7 | 9.0 | 2.4 | 3.9 | 2.7 | 1.7 | 1.0 | 3.8 | 4.6 |
| Cirrh mean | 1.8 | 6.6 | 10.7 | 7.6 | 6.7 | 2.8 | 4.7 | 2.4 | 8.3 | 5.3 | 4.5 |
| Cirrh. median | 0.7 | 3.4 | 9.5 | 4.5 | 5.0 | 3.0 | 2.6 | 2.3 | 5.9 | 3.7 | 4.1 |
| Cirrh. Std. | 2.9 | 6.4 | 8.7 | 7.5 | 6.0 | 1.0 | 4.0 | 1.1 | 8.0 | 4.3 | 2.9 |
| deviation | | | | | | | | | | | |
| Adenoma | 1.9 | 10.0 | 1.7 | 6.9 | 1.6 | 3.6 | 1.8 | 1.1 | 2.2 | 1.5 | 3.7 |
| Copper tox, | 2.3 | 18.7 | 3.5 | 7.2 | 7.0 | 8.4 | 13.0 | 7.3 | 35.5 | 22.4 | 9.5 |
| Non-dis. liver | 0.7 | 0.6 | n,d, | 2.6 | 1.4 | 1.5 | 1.7 | 1.6 | 1.1 | 2.0 | 1.2 |

TABLE 3B
c DNA microarray nucleic acid expression level ratios (log2 values)

| | OBcl1 | OBcl5 | IK2 | IK5 | DAP3 | LOC5 | SEC14L2 | SSP29 | HS16 | IK3 |
|---|---|---|---|---|---|---|---|---|---|---|
| Disease | SEQ | SEQ | SEQ | SEQ | SEQ | SEQ | SEQ | SEQ | SEQ | SEQ |
| sample | ID 10 | ID 11 | ID 12 | ID 13 | ID 14 | ID 15 | ID 16 | ID 17 | ID 18 | ID 19 |
| HCC11 | 1.99 | 4.79 | 1.24 | 3.13 | 1.12 | 0.05 | 3.96 | 1.02 | 0.14 | 0.57 |
| HCC12 | 0.55 | 5.26 | 0.19 | 3.52 | 1.64 | 2.10 | 3.80 | 1.93 | 0.90 | 1.08 |
| HCC13 | 0.46 | 5.48 | 2.97 | 2.74 | 2.87 | 2.82 | 1.73 | 1.05 | 0.81 | 1.50 |
| HCC15 | 1.36 | 4.93 | 0.50 | 1.89 | 4.58 | 2.53 | 1.95 | 3.11 | 0.91 | 0.76 |
| HCC1 | 1.24 | 5.34 | 2.92 | 3.26 | 0.83 | 2.82 | 3.23 | 2.47 | 0.64 | 3.68 |
| HCC27 | 3.53 | 3.56 | 2.06 | 1.34 | 2.64 | 3.04 | 2.21 | 2.32 | 3.20 | 2.82 |
| HCC29 | 3.45 | 4.52 | 3.75 | 1.96 | 2.75 | 2.43 | 0.91 | 2.81 | 2.22 | 1.75 |
| HCC2 | 1.15 | 5.37 | 3.05 | 2.43 | 1.29 | 3.16 | 1.21 | 0.94 | 0.88 | 3.65 |
| HCC30n | 0.96 | 4.57 | −0.19 | 4.40 | 1.31 | 1.84 | 2.46 | 3.38 | 0.77 | −0.38 |
| HCC31 | 0.42 | 3.27 | −0.63 | 3.79 | 1.55 | 1.57 | −0.12 | 1.74 | 1.85 | −0.15 |
| HCC32 | 1.48 | 2.96 | 2.15 | 2.59 | 1.77 | 1.81 | 2.13 | 1.59 | 2.22 | 1.68 |
| HCC33 | −0.17 | 2.83 | 0.97 | 2.05 | 0.96 | 3.06 | 2.85 | 0.94 | −0.07 | 1.76 |
| HCC34 | 1.54 | 5.59 | 3.23 | 4.44 | 1.93 | 3.69 | 4.03 | 0.16 | 0.44 | 2.92 |
| HCC35 | 1.99 | 1.63 | 2.08 | 2.85 | 2.42 | 2.09 | 1.74 | 2.31 | 2.29 | 1.61 |
| HCC36 | 0.86 | 5.18 | 2.32 | 3.08 | 2.40 | 2.61 | 1.74 | 1.28 | 0.52 | 0.35 |
| HCC4 | 0.77 | 4.42 | 3.05 | 3.94 | 3.41 | 0.97 | −0.13 | 1.35 | 1.73 | 0.87 |
| HCC6 | −0.60 | 3.96 | −0.87 | 0.94 | 0.45 | 0.44 | 0.68 | 1.36 | 2.09 | 0.70 |
| HCC9 | 0.29 | 5.72 | 1.84 | 3.97 | 1.44 | 0.17 | 2.15 | 1.92 | 0.47 | −0.13 |
| IHB-HCC | −0.85 | 4.35 | 3.06 | 3.81 | 4.27 | 3.29 | 0.32 | 1.34 | 2.35 | 2.26 |
| HCC22 | 1.66 | 3.39 | 1.16 | 2.31 | 1.28 | −0.31 | 1.26 | 1.45 | 0.32 | 2.35 |
| HCC28 | −0.75 | 2.41 | 1.22 | 2.49 | 0.42 | −0.62 | 0.45 | 0.86 | 0.12 | 0.90 |
| HCC mean | 1.02 | 4.26 | 1.72 | 2.90 | 1.97 | 1.88 | 1.84 | 1.68 | 1.18 | 1.45 |
| HCC median | 0.96 | 4.52 | 2.06 | 2.85 | 1.64 | 2.10 | 1.74 | 1.45 | 0.88 | 1.50 |
| HCC std. | 1.17 | 1.16 | 1.35 | 0.97 | 1.14 | 1.29 | 1.26 | 0.81 | 0.93 | 1.18 |
| deviation | | | | | | | | | | |
| FNH1 | 1.34 | 2.81 | 3.01 | 3.33 | 2.19 | 3.34 | 2.83 | 1.21 | 2.29 | −0.18 |
| FNH2 | 2.24 | 2.84 | 3.45 | 4.01 | 1.14 | 2.83 | 2.08 | 1.11 | 1.08 | 4.05 |
| FNH3 | 1.58 | 2.07 | 3.25 | 3.53 | 0.59 | 3.27 | 2.58 | 0.06 | 1.06 | 3.30 |
| FNH9 | 1.76 | 3.91 | 2.95 | 3.31 | 0.75 | 1.28 | 1.93 | −0.12 | 0.35 | 2.91 |
| FNH mean | 1.56 | 2.99 | 2.68 | 3.39 | 1.07 | 2.32 | 1.91 | 0.67 | 1.07 | 2.40 |
| FNH median | 1.58 | 2.84 | 3.01 | 3.33 | 0.75 | 2.83 | 2.08 | 1.11 | 1.06 | 2.91 |
| FNH std. deviation | 0.50 | 0.68 | 1.10 | 0.44 | 0.66 | 1.16 | 1.05 | 0.65 | 0.75 | 1.64 |
| Cirrh34b | 2.92 | 4.14 | 2.58 | 2.59 | 3.78 | 3.22 | 1.17 | 4.29 | 3.11 | 2.08 |
| Cirrh5 | −0.97 | 1.42 | 3.69 | 1.42 | 0.24 | 3.36 | 2.01 | 4.00 | 1.01 | 1.97 |
| Cirrh1 | 0.02 | 0.86 | 1.16 | 1.48 | 2.91 | 0.92 | 1.19 | 3.22 | 3.61 | 3.34 |
| Cirrh2 | −1.43 | 1.37 | 1.55 | 1.55 | 3.80 | 1.29 | 1.70 | 0.81 | 0.40 | 1.44 |
| Cirrh3 | −1.28 | 1.99 | 3.93 | 4.47 | 0.40 | 0.53 | −0.40 | 1.28 | 1.85 | 0.74 |
| Cirrh4 | −0.37 | 3.43 | 4.62 | 3.17 | 1.27 | 1.44 | 0.75 | −0.05 | 1.92 | 2.20 |
| Cirrh mean | −0.18 | 2.20 | 2.92 | 2.45 | 2.07 | 1.79 | 1.07 | 2.26 | 1.98 | 1.96 |
| Cirrh median | −0.67 | 1.70 | 3.13 | 2.07 | 2.09 | 1.37 | 1.18 | 2.25 | 1.89 | 2.02 |
| Cirrh std. | 1.62 | 1.30 | 1.39 | 1.22 | 1.63 | 1.20 | 0.84 | 1.82 | 1.22 | 0.86 |
| deviation | | | | | | | | | | |
| Adenoma | 0.89 | 3.32 | 0.75 | 2.78 | 0.70 | 0.87 | 0.15 | 1.11 | 0.56 | 1.89 |
| Copper tox. | 1.21 | 4.23 | 1.82 | 2.85 | 2.82 | 3.70 | 2.87 | 5.15 | 4.48 | 3.25 |
| Non-dis. liver | −0.53 | −0.84 | n.d. | 1.39 | 0.50 | 0.80 | 0.69 | 0.19 | 0.97 | 0.23 |

A summary of cDNA microarray nucleic acid expression values is shown in Table 4. Mann-Whitney-U Test is applied to statistically evaluate RNA expression levels: This test is equal to the Wilcoxon Rank Sum two-sided Test with paired flag=‘false’ (Hollander & Wolfe, 1973, Nonparametric statistical inference. New York: John Wiley & Sons, pgs. 27-33, 68-75; Bauer, D. F., 1972, J. Amer. Statistical Assoc. 67: 687-690). The expression values typically do not fit to a normal distribution so averaging the values may be misleading. However, analysis of the median values demonstrates significant differences in most of the cases between experimental and reference values, particularly in the large data sets. Expt. median=median value for experimental (diseased) tissues; Expt. iqr=experimental value interquartile range (+/−25^{th} percentile of median value); Contr. median=median value for control (non-diseased) tissue samples; Contr. iqr=control value interquartile range (+/−25^{th} percentile of median value); p value=value resulting from statistical evaluation of the probability that the experimental and control values are significantly different.

TABLE 4
Summary of cDNA microarray nucleic acid (SEQ ID 10 to 19) expression
values
HCC

| | Expt. | Expt. | Contr. | Contr. | |
|---|---|---|---|---|---|
| | median | iqr | median | iqr | P value |
| OBcl1 | 6482 | 4915 | 3235 | 1050 | 0.0001 |
| OBCl5 | 995.5 | 1549.1 | 832.2 | 195 | 0.0156 |
| IK2 | 582.7 | 348.9 | 874.3 | 344.1 | 0.0397 |
| IK5 | 600.1 | 330.4 | 760.9 | 261.5 | 0.0056 |
| DAP3 | 1202 | 1271.7 | 927 | 391.3 | 0.0499 |
| LOC5 | 673.7 | 256.2 | 965 | 255.4 | 0.0255 |
| SEC14L2 | 457.39 | 351.17 | 869.7 | 306.1 | 0.0003 |
| SSP29 | 949.9 | 475.1 | 976.2 | 327.9 | 0.6792 |
| HS16 | 1269 | 483 | 1083 | 494.4 | 0.2293 |
| IK3 | 651.7 | 305.2 | 842.2 | 297.3 | 0.0080 |

FNH

| | Expt. | Expt. | Contr. | Contr. | |
|---|---|---|---|---|---|
| | median | iqr | median | iqr | P value |
| OBcl1 | 8279.2 | 3205 | 3550.1 | 684 | 0.0286 |
| OBCl5 | 806.4 | 1563.4 | 737.6 | 106.5 | 0.4857 |
| IK2 | 1165.1 | 222 | 887.2 | 137 | 0.6857 |
| IK5 | 1358.9 | 383 | 882.1 | 196.6 | 0.4857 |
| DAP3 | 1555.6 | 569 | 1046.2 | 136 | 0.3429 |
| LOC5 | 971.3 | 459.3 | 890.7 | 131 | 0.6857 |
| SEC14L2 | 807.3 | 262.9 | 806 | 176.6 | 0.6857 |
| SSP29 | 1484.4 | 462 | 1139.9 | 101 | 0.2000 |
| HS16 | 1556.2 | 644 | 1156.5 | 113 | 0.4857 |
| IK3 | 1298.9 | 131 | 800.7 | 360.4 | 0.3429 |

Cirrhosis

| | Expt. | Expt. | Contr. | Contr. | |
|---|---|---|---|---|---|
| | median | iqr | Median | iqr | P value |
| OBcl1 | 2518 | 1923 | 4108 | 869 | 0.2403 |
| OBCl5 | 318.4 | 187 | 1318 | 321 | 0.0087 |
| IK2 | 408.3 | 235 | 1195 | 194 | 0.0022 |
| IK5 | 244 | 251.7 | 1238 | 995 | 0.0022 |
| DAP3 | 576.1 | 568.1 | 1417 | 446 | 0.0022 |
| LOC5 | 355.6 | 360 | 1377 | 293 | 0.0022 |
| SEC14L2 | 192.3 | 112.8 | 1287 | 243 | 0.0022 |
| SSP29 | 361.3 | 140.4 | 1547 | 501 | 0.0087 |
| HS16 | 246.7 | 250.5 | 1392 | 300 | 0.0022 |
| IK3 | 378.6 | 446.6 | 1217 | 423 | 0.0043 |

Comparison of nucleic acid expression values in non-neoplastic liver diseases and liver cancer is shown in Table 5A. For each nucleic acid according to the invention a P value is provided for the difference in the median experimental expression values for comparisons between FNH, Cirrh. and HCC samples. For each nucleic acid and comparison a P value of less than or equal to 0.05 indicates a significant difference in expression values between the disease groups. Significance was assessed with the Wilcoxon rank sum test. Statistically significant differences in expression are evident between disease groups. For example, the expression values for IK2 are significantly different in all three comparisons (P values less than 0.05). The FNH sample group is small and displayed a large distribution of values. This likely accounts for fewer significant differences in comparisons with this group.

TABLE 5A
Expression specificity of nucleic acid (SEQ ID 10 to 19) in HCC
vs. Cirrh; HCC vs. FNH; Cirrh vs. FNH

| | HCC vs. Cirrh. | HCC vs. FNH | Cirrh. vs. FNH |
|---|---|---|---|
| OBcl1 | 0.0013 | 0.2718 | 0.0095 |
| OBcl5 | 0.0010 | 0.7672 | 0.0667 |
| IK2 | 0.0042 | 0.0081 | 0.0095 |
| IK5 | 0.0078 | 0.0031 | 0.0095 |
| DAP3 | 0.0078 | 0.4885 | 0.0667 |
| LOC5 | 0.0042 | 0.1109 | 0.0095 |
| SEC14L2 | 0.0004 | 0.0817 | 0.0095 |
| SSP29 | 0.0052 | 0.0336 | 0.0095 |
| HS16 | 0.0168 | 0.4085 | 0.0095 |
| IK3 | 0.1273 | 0.0014 | 0.0095 |

Mann-Whitney U in Table 5B indicates the number of times a value in the first group (HCC) exceeds a value in the second group (FHN and Cirrh respectively), when values are sorted in ascending order. Wilcoxon W is the sum of ranks for the larger of the two groups in the Mann-Whitney Wilcoxon Rank Sum Test. Asymptotic Significance (Asymp. Sig.) (2 tailed) provides a P-value for two-sided test. This statistic analysis is employed to determine an overall trend of expression patern of OBcl5 (HCC vs FNH, HCC vs Cirrh) verified by statistics of quantitative RT PCR (Q-PCR) data provided in Table 7 and shown in FIG. 2.

TABLE 5B
Expression specificity of OBcl5 in HCC vs. FNH and HCC vs. Cirrh

| | | |
|---|---|---|
| | | Asymp. Sig. |
| Mann-Whitney U | Wilcoxon W | (2-tailed) |

HCC vs. FNH

| | | | |
|---|---|---|---|
| OBCl5 | 18.0 | 33.0 | 0.025 |

HCC vs Cirrh

| | | | |
|---|---|---|---|
| OBCl5 | 15.0 | 36.0 | 0.005 |

Reverse transcriptase polymerase chain reaction (RT-PCR) is performed with primers specific for each deregulated nucleic acid in each tissue listed to determine if the sequence is represented in RNA prepared from each tissue. All tissues employed are diagnostically confirmed prior to utilization for RNA (and cDNA) preparation. In Table 6 the “+” symbol indicates that the gene is expressed in the tissue, the “−” indicates that this gene is not detected in cDNA from this RNA sample; and a blank box indicates that the analysis is not performed for that gene and tissue combination. The patient's age and sex is provided. Additional sample information includes the tumor staging value (T=tumor size), as well as the tumor grading score (G=tumor cell differentiation); large numbers indicate larger and less well-differentiated tumors, respectively. The positive control for tissue cDNA is amplification from the glyceraldehyde phosphate dehydrogenase mRNA (GAPDH).

TABLE 6
RT-PCR analysis of nucleic acid expression in human non-diseased and disease tissues

| | | Pa- | | | | | | | | | | | | | | |
|---|---|---|---|---|---|---|---|---|---|---|---|---|---|---|---|---|
| | Pateint | tient | | | | | | | | | | | | | | |
| sample | sex | age | diagnosis | T | G | GAPDH | OBcl1 | OBcl5 | IK2 | IK5 | DAP3 | LOC5 | SEC14L2 | SSP29 | HS16 | IK3 |
| liver | m | 45 | non- | | | + | | | | + | − | | | | | |
| | | | diseased | | | | | | | | | | | | | |
| | | | tissue | | | | | | | | | | | | | |
| liver | m | 27 | non- | | | + | − | − | − | − | − | − | − | − | − | − |
| | | | diseased | | | | | | | | | | | | | |
| | | | tissue | | | | | | | | | | | | | |
| liver | | | non- | | | + | | | | + | − | | | | | |
| | | | diseased | | | | | | | | | | | | | |
| | | | tissue | | | | | | | | | | | | | |
| HCC1 | m | 66 | trabecular/ | 3 | 1 | + | − | + | + | | | | | | | + |
| | | | tubular | | | | | | | | | | | | | |
| | | | HCC | | | | | | | | | | | | | |
| HCC2 | m | 81 | trabecular/ | 3 | 2 | + | − | + | + | + | | | | | | |
| | | | tubular | | | | | | | | | | | | | |
| | | | HCC | | | | | | | | | | | | | |
| HCC3 | m | 63 | trabecular/ | 3 | 2 | + | − | + | − | − | | − | − | − | | − |
| | | | tubular | | | | | | | | | | | | | |
| | | | HCC | | | | | | | | | | | | | |
| HCC4 | m | 72 | trabecular/ | 3 | 2 | + | − | +/− | | | | | | | | |
| | | | tubular | | | | | | | | | | | | | |
| | | | HCC | | | | | | | | | | | | | |
| Ade- | f | 22 | benign | | | + | − | + | + | + | | + | | +/− | | + |
| noma | | | liver | | | | | | | | | | | | | |
| | | | neoplasm | | | | | | | | | | | | | |
| HCC | m | 63 | trabecular/ | 2 | | + | − | + | | | | | | | | |
| (from | | | tubular | | | | | | | | | | | | | |
| HCV) | | | HCC | | | | | | | | | | | | | |
| HCC | | | | | | | + | + | + | − | | + | + | − | + | − |
| cDNA | | | | | | | | | | | | | | | | |
| libr. | | | | | | | | | | | | | | | | |
| Pool | | | | | | | | | | | | | | | | |
| colon | m | 52 | non- | | | + | − | + | − | − | + | − | − | − | | − |
| | | | disease | | | | | | | | | | | | | |
| | | | tissue | | | | | | | | | | | | | |
| colon | m | 69 | tubular | 4 | 2 | + | − | − | − | − | +/− | − | − | + | | − |
| tumor | | | adeno- | | | | | | | | | | | | | |
| | | | carcinoma | | | | | | | | | | | | | |
| colon | m | 64 | tubular | 3 | 2 | + | − | − | + | + | + | + | + | − | | − |
| tumor | | | adeno- | | | | | | | | | | | | | |
| | | | carcinoma | | | | | | | | | | | | | |
| colon | m | 52 | tubular | 3 | 2 | + | − | − | | | | | | | | |
| tumor | | | adeno- | | | | | | | | | | | | | |
| | | | carcinoma | | | | | | | | | | | | | |
| | | Pa- | | | | | | | | | | | | | | |
| | | tient | | | | | | | | | | | | | | |
| sample | Pateint | age | diagnosis | T | G | GAPDH | OBcl1 | OBcl5 | IK2 | IK5 | DAP3 | LOC5 | SEC14L2 | SSP29 | HS16 | IK3 |
| stomach | f | 57 | non- | | | + | − | + | | | +/− | | | | | |
| | | | diseased | | | | | | | | | | | | | |
| | | | tissue | | | | | | | | | | | | | |
| stomach | m | 70 | non- | | | | − | − | − | − | | − | − | − | | − |
| | | | diseased | | | | | | | | | | | | | |
| | | | tissue | | | | | | | | | | | | | |
| stomach | f | 61 | adeno- | 2 | | + | − | + | | | + | | | | | |
| tumor | | | carcinoma | | | | | | | | | | | | | |
| stomach | f | 78 | adeno- | 3 | 3 | + | +/− | − | | | − | | | | | |
| tumor | | | carcinoma | | | | | | | | | | | | | |
| stomach | f | 70 | tubular | X | 3 | + | − | − | | | − | | | | | |
| tumor | | | adeno- | | | | | | | | | | | | | |
| | | | carcinoma | | | | | | | | | | | | | |
| stomach | m | 69 | adeno- | 3 | 3 | + | − | − | | | | | | | | |
| tumor | | | carcinoma | | | | | | | | | | | | | |
| pan- | m | 55 | non- | | | + | − | − | − | + | +/− | − | − | − | | − |
| creas | | | diseased | | | | | | | | | | | | | |
| | | | tissue | | | | | | | | | | | | | |
| pan- | m | 69 | adeno- | 3 | 3 | + | − | + | | | − | | | | − | |
| creas | | | carcinoma | | | | | | | | | | | | | |
| tumor | | | | | | | | | | | | | | | | |
| pan- | m | 69 | adeno- | 3 | 3 | + | − | − | | | | | | | | |
| creas | | | carcinoma | | | | | | | | | | | | | |
| tumor | | | | | | | | | | | | | | | | |
| skin | f | 60 | non- | | | + | − | − | | | − | | | | | |
| | | | diseased | | | | | | | | | | | | | |
| | | | tissue | | | | | | | | | | | | | |
| skin | m | 50 | squamous | | 2 | + | − | − | | | | | | | | |
| tumor | | | cell | | | | | | | | | | | | | |
| | | | carcinoma | | | | | | | | | | | | | |
| skin | f | 92 | squamous | 2 | 3 | + | − | − | | | − | | | | | |
| tumor | | | cell | | | | | | | | | | | | | |
| | | | carcinoma | | | | | | | | | | | | | |
| skin | m | 73 | squamous | 2 | 1 | + | − | − | | | + | | | | | |
| tumor | | | cell | | | | | | | | | | | | | |
| | | | carcinoma | | | | | | | | | | | | | |
| testis | m | 48 | non- | | | + | − | + | − | − | − | − | − | − | | − |
| | | | diseased | | | | | | | | | | | | | |
| | | | tissue | | | | | | | | | | | | | |
| testis | m | 35 | seminoma | 3 | | + | − | − | − | − | + | − | − | − | | − |
| tumor | | | and yolk | | | | | | | | | | | | | |
| | | | sac tumor | | | | | | | | | | | | | |
| testis | m | 43 | seminoma | 2 | | + | − | − | | | − | | | | | |
| tumor | | | | | | | | | | | | | | | | |
| testis | m | 31 | seminoma | 1 | | + | − | − | | | − | | | | | |
| tumor | | | | | | | | | | | | | | | | |
| thyroid | f | 60 | papillary | 3a | | + | − | − | − | − | +/− | − | − | − | − | − |
| tumor | | | carcinoma | | | | | | | | | | | | | |
| thyroid | f | 57 | papillary | 4a | | + | − | + | − | − | | − | − | − | − | − |
| tumor | | | carcinoma | | | | | | | | | | | | | |
| thyroid | f | 17 | papillary | 2b | | + | − | − | − | − | + | − | − | − | − | − |
| tumor | | | carcinoma | | | | | | | | | | | | | |
| kidney | f | 33 | non- | | | + | − | − | + | − | − | − | − | − | − | − |
| | | | diseased | | | | | | | | | | | | | |
| | | | tissue | | | | | | | | | | | | | |
| kidney | f | 33 | clear | 1 | 1 | + | − | − | | | + | | | | | |
| tumor | | | cell | | | | | | | | | | | | | |
| | | | carcinoma | | | | | | | | | | | | | |
| kidney | f | 62 | clear | 1 | 1 | − | − | + | − | − | − | − | − | − | | − |
| tumor | | | cell | | | | | | | | | | | | | |
| | | | carcinoma | | | | | | | | | | | | | |
| kidney | m | 54 | clear | 1 | 2 | + | − | − | + | + | +/− | − | + | − | − | + |
| tumor | | | cell | | | | | | | | | | | | | |
| | | | carcinoma | | | | | | | | | | | | | |
| lung | f | 64 | non- | | | + | − | + | − | − | − | − | − | − | − | − |
| | | | diseased | | | | | | | | | | | | | |
| | | | tissue | | | | | | | | | | | | | |
| lung | f | 57 | non- | | | + | − | + | − | − | − | − | − | − | − | − |
| | | | diseased | | | | | | | | | | | | | |
| | | | tissue | | | | | | | | | | | | | |
| lung | m | 58 | squamous | 2 | 3 | + | − | − | + | + | +/− | − | − | − | − | + |
| tumor | | | cell | | | | | | | | | | | | | |
| | | | carcinoma | | | | | | | | | | | | | |
| lung | m | 54 | squamous | 2 | 2-3 | + | − | + | +/− | − | − | − | − | − | − | + |
| tumor | | | cell | | | | | | | | | | | | | |
| | | | carcinoma | | | | | | | | | | | | | |
| lung | f | 57 | squamous | 2 | 2-3 | + | − | − | − | − | − | − | − | − | − | − |
| tumor | | | cell | | | | | | | | | | | | | |
| | | | carcinoma | | | | | | | | | | | | | |
| mam- | f | 38 | non- | | | + | − | − | +/− | − | +/− | − | + | − | − | − |
| mary | | | diseased | | | | | | | | | | | | | |
| gland | | | tissue | | | | | | | | | | | | | |
| mam- | f | 55 | invasive | 2 | 2 | + | − | − | + | − | | − | − | − | − | − |
| mary | | | ductal | | | | | | | | | | | | | |
| tumor | | | carcinoma | | | | | | | | | | | | | |
| | | | (IDC) | | | | | | | | | | | | | |
| mam- | f | 66 | muscinous | 2 | 1 | + | − | + | − | − | − | − | − | − | − | − |
| mary | | | carcinoma | | | | | | | | | | | | | |
| tumor | | | | | | | | | | | | | | | | |
| spleen | f | 58 | non- | | | + | − | − | − | − | − | − | − | − | − | − |
| | | | diseased | | | | | | | | | | | | | |
| | | | tissue | | | | | | | | | | | | | |
| muscle | m | 65 | non- | | | + | − | − | − | − | − | − | − | − | − | − |
| | | | diseased | | | | | | | | | | | | | |
| | | | tissue | | | | | | | | | | | | | |
| brain | m | 27 | non- | | | + | − | + | − | − | − | − | − | − | − | − |
| (cortex) | | | diseased | | | | | | | | | | | | | |
| | | | tissue | | | | | | | | | | | | | |
| brain | m | 27 | non- | | | + | − | − | − | − | − | + | − | + | − | − |
| medulla | | | diseased | | | | | | | | | | | | | |
| | | | tissue | | | | | | | | | | | | | |
| heart | | | non- | | | | | | | | + | | | | | + |
| | | | diseased | | | | | | | | | | | | | |
| | | | tissue | | | | | | | | | | | | | |
| bone | | | non- | | | | | | | + | | + | | | | + |
| marrow | | | diseased | | | | | | | | | | | | | |
| | | | tissue | | | | | | | | | | | | | |
| placenta | | | non- | | | + | + | − | + | + | | + | + | + | | + |
| cDNA | | | diseased | | | | | | | | | | | | | |
| library | | | tissue | | | | | | | | | | | | | |

In another preferred embodiment of the invention the nucleic acid according to the invention can be used for the construction of antisense oligonucleotides (Zheng and Kemeny, 1995, Clin. Exp. Immunol. 100: 380-2; Nellen and Lichtenstein, 1993, Trends Biochem. Sci. 18: 419-23; Stein, 1992, Leukemia 6: 967-74) and/or ribozymes (Amarzguioui, et al. 1998, Cell. Mol. Life Sci. 54: 1175-202; Vaish et al., 1998, Nucleic Acids Res. 26: 5237-42; Persidis, 1997, Nat. Biotechnol. 15: 921-2; Couture and Stinchcomb, 1996, Trends Genet. 12: 510-5) and/or small interfering double stranded RNAs (Elbashir et al., 2001, Nature 411: 494-98; Brummelkamp et al., 2002, Science 296:550-553). In further preferred embodiments of the invention, the stability of the nucleic acid according to the invention can be decreased and/or the translation of the nucleic acid according to the invention inhibited by using RNA interference molecules (oligonucleotides). Thus, for example, the expression of the corresponding genes in cells can be decreased both in vivo and in vitro. Oligonucleotides can therefore be suitable as therapeutics. This strategy is also suitable, for example, for liver cells, in particular if the antisense oligonucleotides are complexed with liposomes. For use as a probe or as an “antisense” oligonucleotide, a single-stranded DNA or RNA is preferred. Small interfering RNA (siRNA) double stranded oligonucleotides can also be suitable as therapeutics. With this approach a short sequence or sequences of 15 to 22 nucleotides including sequence complimentary to the sequence to be therapeutically targeted are exposed to the diseased tissue and serve to dramatically reduce or “knock down” the level of expression of the therapeutic target RNA sequence. siRNA therapeutic approaches in other diseases have been recently reported and are also applicable to liver disorders, liver cancers and other epithelial cancers (Filleur S, Courtin A, Ait-Si_Ali S, Guglielmi J, Merle C, Harel-Bellan A, Clezardin P, Cabon F. Cancer Res. 2003 July 15; 63 (14): 39-22.).

In a preferred embodiment a nucleic acid according to the invention has been prepared by recombinant methods, by screening a library or isolation from a sample obtained from a patient or a subject. In another preferred embodiment of the invention the nucleic acid according to the invention has been prepared synthetically. Thus, the nucleic acid according to the invention can be synthesized, for example, chemically with the aid of the DNA sequences described in SEQ ID 10 to SEQ ID 19 and/or with the aid of the protein sequences described in SEQ ID 1 to SEQ ID 9 and/or ID SEQ 47 with reference to the genetic code, e.g. according to the phosphotriester method (see, for example, Uhlmann and Peyman, 1990, Chemical Reviews 90:543-584).

In another preferred embodiment, the invention relates to a nucleic acid according to the invention or a nucleic acid which is a non-functional mutant variant the nucleic acid or a nucleic acid having a sequence complementary to one of the aforementioned nucleic acids, which has been modified by attachment of chemical moieties to the nucleic acid to stabilize it against degradation, so that a high concentration of the nucleic acid is maintained in the cell over a long period (Beigelman et al., 1995, Nucleic Acids Res. 23: 3989-94; Dudycz, 1995, WO 95/11910; Macadam et al., 1998, WO 98/37240; Reese et al., 1997, WO 97/29116). Typically, such stabilization can be obtained by the introduction of one or more internucleotide phosphorus groups or by the introduction of one or more non-phosphorus internucleotides.

Preferred suitable modified internucleotides are summarized in Uhlmann and Peymann (1990 Chem. Rev. 90, 544; see also Beigelman et al., 1995 Nucleic Acids Res. 23: 3989-94; Dudycz, 1995, WO 95/11910; Macadam et al., 1998, WO 98/37240; Reese et al., 1997, WO 97/29116).

In a further embodiment the invention relates to a vector comprising a nucleic acid according to the invention and/or a variant thereof, or a nucleic acid which is a non-functional mutant variant of the nucleic acid, or a nucleic acid having a sequence complementary to one the aforementioned nucleic acids. Preferably the vector is a knock-out gene construct, a plasmid, a shuttle vector, a phagemid, a cosmid, a viral vector, an expression vector and/or a vector applicable in gene therapy. The preparation of such constructs is generally known to the person skilled in the art.

An “expression vector” within the meaning of the present invention preferably comprises at least one promoter or enhancer, i.e. at least one regulatory element comprising at least one translation initiation signal, at least one of the nucleic acids according to the invention or a nucleic acid which is a non-functional mutant variant the nucleic acid or a nucleic acid having a sequence complementary to one of the aforementioned nucleic acids, one translation termination signal, a transcription termination signal, and a polyadenylation signal for the expression in eukaryotes.

For the expression of the gene concerned, in general a double-stranded DNA is preferred, the DNA region coding for the polypeptide being particularly preferred. In the case of eukaryotes this region begins with the first start codon (ATG) lying in a Kozak sequence (Kozak, 1987, Nucleic. Acids Res. 15: 8125-48) up to the next stop codon (TAG, TGA or TAA), which lies in the same reading frame to the ATG. In the case of prokaryotes this region begins with the first AUG (or GUG) after a Shine-Dalgarno sequence and ends with the next stop codon (TAA, TAG or TGA), which lies in the same reading frame to the ATG.

Differentially expressed genes in HCC can contain liver or liver cancer gene-specific regulatory sequences. These non-transcribed sequences, found in the tissue- or disease-specific gene may be used to drive tissue- or disease-specific expression of included therapeutic and/or tumor cell-cytotoxic genes. These regulatory sequences may be used for liver cancer specific expression of a nucleic acid according to the invention or a nucleic acid which is a non-functional mutant variant the nucleic acid or a nucleic acid having a sequence complementary to one of the aforementioned nucleic acids. The screening and construction of such regulatory sequences is generally known to the person skilled in the art.

Suitable expression vectors can be prokaryotic or eukaryotic expression vectors. Examples of prokaryotic expression vectors are, for expression in E. coli, e.g. the vectors pGEM or pUC derivatives, examples of eukaryotic expression vectors are for expression in Saccharomyces cerevisiae, e.g. the vectors p426Met25 or p426GAL1 (Mumberg et al. (1994) Nucl. Acids Res., 22, 5767-5768), for expression in insect cells, e.g. Baculovirus vectors such as disclosed in EP-B1-0 127 839 or EP-B1-0 549 721, and for expression in mammalian cells, e.g. the vectors Rc/CMV and Rc/RSV or SV40 vectors, which are all generally obtainable. Specific vectors for production of RNA interference following transfection, such as the pSUPER vector (Brummelkamp et al., 2002, Science 296:550-553) are also included.

In general, the expression vectors also contain promoters suitable for the respective cell, such as, for example, the trp promoter for expression in E. coli (see, for example, EP-B 1-0 154 133), the MET 25, GAL 1 or ADH2 promoter for expression in yeast (Russel et al. (1983), J. Biol. Chem. 258, 2674-2682; Mumberg, supra), the Baculovirus polyhedrin promoter, for expression in insect cells (see, for example, EP-B 1-0 127 839). For expression in mammalian cells, for example, suitable promoters are those which allow a constitutive, regulatable, tissue-specific, cell-cycle-specific or metabolically specific expression in eukaryotic cells. Regulatory elements according to the present invention preferably are promoters, activator sequences, enhancers, silencers and/or repressor sequences.

Examples of suitable regulatory elements which make possible constitutive expression in eukaryotes preferably are promoters which are recognized by the RNA polymerase III or viral promoters, CMV enhancer, CMV promoter, SV40 promoter or LTR promoters, e.g. from MMTV (mouse mammary tumor virus; Lee et al. (1981) Nature 214, 228-232) and further viral promoter and activator sequences, derived from, for example, adeno- and adeno-like viruses, HBV, HCV, HSV, HPV, EBV, HTLV or HIV.

Examples of regulatory elements which make possible regulated expression in eukaryotes are the tetracycline operator in combination with a corresponding repressor (Gossen et al., 1994, Curr. Opin. Biotechnol. 5: 516-20).

Translation initiation signals, translation termination signals, transcription termination signals, and polyadenylation signals are generally known to the person skilled in the art and can be readily obtained from commercial laboratory suppliers.

Preferably, the expression of the genes relevant for liver disorders and/or epithelial cancer takes place under the control of tissue-specific promoters, for example, under the control of liver-specific promoters such as albumin, alpha fetoprotein, apolipoprotein AI, alpha-I antitrypsin, and the complement C5 and C8A genes (Schrem et al., 2002, Pharmacol. Rev. 54 129-58; Pontoglio et al., 2001, J. Expt. Med. 194:1683-1689). The regulatory sequences associated with genes highly deregulated in HCC as described herein also provide a preferable method for specific gene expression in these disorders.

Further examples of regulatory elements which make tissue-specific expression in eukaryotes possible are promoters or activator sequences from promoters or enhancers of those genes which code for proteins which are only expressed in certain cell types.

Examples of regulatory elements which make possible metabolically specific expression in eukaryotes are promoters which are regulated by hypoxia, by oxidative stress, by glucose deficiency, by phosphate concentration or by heat shock.

Examples of regulatory elements which make cell cycle-specific expression in eukaryotes possible are promoters of the following genes: cdc25A, cdc25B, cdc25C, cyclin A, cyclin E, cdc2, E2F-1 to E2F-5, B-myb or DHFR (Zwicker J. and Müller R. 1997, Trends Genet. 13: 3-6). The use of cell cycle regulated promoters is particularly preferred in cases, in which expression of the polypeptides or nucleic acids according to the invention is to be restricted to proliferating cells.

In order to make possible the introduction of nucleic acids as described above, or a nucleic acid which is a non-functional mutant variant of the nucleic acid and thus the expression of the polypeptide in a eukaryotic or prokaryotic cell by transfection, transformation or infection, the nucleic acid can be present as a plasmid, as part of a viral or non-viral vector. Suitable viral vectors here are particularly: baculoviruses, vaccinia viruses, adenoviruses, adeno-associated viruses, retroviruses and herpesviruses. Suitable non-viral vectors here are particularly: virosomes, liposomes, cationic lipids, or polylysine-conjugated DNA or naked DNA.

Plasmids, shuttle vectors, phagemids, and cosmids suitable for use according to the invention are also known to the person skilled in the art and are generally obtainable from commercial laboratory suppliers.

Examples of vectors applicable in gene therapy are virus vectors, for example adenovirus vectors, retroviral vectors or vectors based on replicons of RNA viruses (Lindemann et al., 1997, Mol. Med. 3: 466-76; Springer et al., 1998, Mol. Cell. 2: 549-58, Khromykh, 2000, Curr. Opin. Mol Ther. 2:555-569). Eukaryotic expression vectors are suitable in isolated form for gene therapy use, as naked DNA can penetrate, for example, into liver cells upon local application or via the blood supply.

Compared to the state of the art, this fusion construct surprisingly allows improved, more sensitive, earlier, faster, and/or non-invasive diagnosis and/or improved, sustained and/or more effective treatment of the liver disorders, and/or other epithelial cancers.

In another aspect the invention furthermore relates to a cell comprising a nucleic acid according to the invention and/or a variant thereof. Preferably the cell is transformed with a vector according to the invention. The cell preferably contains a nucleic acid wherein the nucleic acid is either a non-functional mutant variant of a nucleic acid according to the invention, or a variant thereof. In particular the cell contains vector comprising a nucleic acid wherein the nucleic acid is a non-functional mutant variant of a nucleic acid according to the invention, or a variant thereof. Perferably the cell contains a nucleic acid coding for a nucleic acid having a sequence complementary to a nucleic acid according to the invention, or a variant thereof. Moreover the cell preferably contains a vector comprising a nucleic acid coding for an antibody according to the invention or a fragment of the antibody. The cell according to the invention may for example be a liver cell, comprising at least one of the aforementioned nucleic acids or a cell which is transformed using one of the above described vectors. Cells can be either prokaryotic or eukaryotic cells, heterologous or autologous cells. Examples of prokaryotic cells are E. coli and examples of eukaryotic cells include primary hepatocytes cells, hepatocytes cell lines such as HepG2 and Hep3B cells, yeast cells, for example Saccharomyces cerevisiae or insect cells.

Compared to the state of the art, the cell according to the invention surprisingly allows improved, more sensitive, earlier, faster, and/or non-invasive diagnosis and/or improved, sustained and/or more effective treatment of the liver disorders and/or other epithelial cancers.

In a preferred embodiment of the invention the cell is a transgenic embryonic non-human stem cell which comprises at least one nucleic acid according to the invention, at least one vector, at least one knock-out gene construct and/or at least one expression vector as described above.

Processes for the transformation of cells and/or stem cells are well known to a person skilled in the art and include, for example, electroporation or microinjection.

In another aspect the invention relates to the provision of a transgenic non-human mammal comprising a compound selected from the group consisting of a nucleic acid according to the invention and/or a variant thereof, a nucleic acid which is a non-functional mutant variant the nucleic acid, a nucleic acid having a sequence complementary to one of the aforementioned nucleic acids, one of the aformementioned nucleic acids in the form of a vector, of a knock-down or knock-out gene construct, and of an expression vector.

Transgenic animals in general show a tissue-specifically increased expression of the nucleic acids and/or polypeptides and can be used for the analysis of liver disorders and/or epithelial cancers, such as for example HCC, and for development and evaluation of therapeutic strategies for such disorders. Transgenic animals may further be employed in the production of polypeptides according to the invention. The polypeptide produced by the animal may for example be enriched in a body fluid of the animal. The polypeptides according to the invention may for example be isolatable from a body fluid such as the milk.

Compared to the state of the art, this transgenic non-human mammal surprisingly allows improved, more sensitive, earlier, faster, and/or non-invasive analysis and/or diagnosis of liver disorders and/or other epithelial cancers.

Processes for the preparation of transgenic animals, in particular of transgenic mice, are likewise known to the person skilled in the art from DE 196 25 049 and U.S. Pat. No. 4,736,866; U.S. Pat. No. 5,625,122; U.S. Pat. No. 5,698,765; U.S. Pat. No. 5,583,278 and U.S. Pat. No. 5,750,825 and include transgenic animals which can be produced, for example, by means of direct injection of expression vectors according to the invention into embryos or spermatocytes or by injection of the expression vectors into the pronucleus of the fertilized ovum or by means of the transfection of expression vectors into embryonic stem cells or by nuclear transfer into appropriate recipient cells (Polites and Pinkert, DNA Microinjection and Transgenic Animal Production, page 15 to 68 in Pinkert, 1994, Transgenic animal technology: a laboratory handbook, Academic Press, London, UK; Houdebine, 1997, Harwood Academic Publishers, Amsterdam, The Netherlands; Doetschman, Gene Transfer in Embryonic Stem Cells, page 115 to 146 in Pinkert, 1994, supra; Wood, Retrovirus-Mediated Gene Transfer, page 147 to 176 in Pinkert, 1994, supra; Monastersky, Gene Transfer Technology; Alternative Techniques and Applications, page 177 to 220 in Pinkert, 1994, supra).

If the above described nucleic acids are integrated into so-called “targeting vectors” or “knock-out” gene constructs (Pinkert, 1994, supra), it is possible after transfection of embryonic stem cells and homologous recombination, for example, to generate knock-out mice which, in general, as heterozygous mice, show decreased expression of the nucleic acid, while homozygous mice no longer exhibit expression of the nucleic acid. The animals thus produced can also be used for the analysis of liver disorders, such as for example HCC, and/or epithelial cancers.

Knock-out gene constructs are known to the person skilled in the art, for example, from the U.S. Pat. No. 5,625,122; U.S. Pat. No. 5,698,765; U.S. Pat. No. 5,583,278 and U.S. Pat. No. 5,750,825.

In a further aspect the invention relates to an antibody or a fragment thereof is provided, wherein the antibody or antibody fragment is directed against a polypeptide according to the invention, a functional variant thereof or against a nucleic acid coding for the polypeptide, or a variant thereof.

Compared to the state of the art, these antibody or a fragment thereof surprisingly allow improved, more sensitive, earlier, faster, and/or non-invasive diagnosis and/or improved, sustained and/or more effective treatment of the liver disorders and/or other epithelial cancers.

The term “antibody” or “antibody fragment” is understood according to the present invention as also meaning antibodies or antigen-binding parts thereof prepared by genetic engineering and optionally modified, such as, for example, chimeric antibodies, humanized antibodies, multifunctional antibodies, bi- or oligospecific antibodies, single-stranded antibodies, F(ab) or F(ab)₂ fragments (see, for example, EP-B1-0 368 684, U.S. Pat. No. 4,816,567, U.S. Pat. No. 4,816,397, WO 88/01649, WO 93/06213, WO 98/24884). The antibodies according to the invention can for example be used for diagnosis, prevention and/or treatment of disorders according to the invention such as liver disorders, for example HCC, and/or epithelial cancers.

The invention further relates to a method for producing an antibody or antibody fragment, preferably a polyclonal or monoclonal antibody, specific for the polypeptides or functional variants thereof encoded by the nucleic acids according to the invention, or variants thereof for example for the diagnosis and/or prevention and/or treatment of disorders according to the invention. The process is carried out according to methods generally known to the person skilled in the art by immunizing a mammal, for example a rabbit, with a nucleic acid according to the invention or their variants thereof, or with a polypeptide according to the invention or parts thereof or functional variants thereof, having at least 6 amino acid length, preferably having at least 8 amino acid length, in particular having at least 12 amino acid length, if appropriate in the presence of, for example, Freund's adjuvant and/or aluminum hydroxide gels (see, for example, Harlow and Lane, 1998, Using Antibodies: A Laboratory Manual, Cold Spring Harbor Press, New York, USA, Chapter 5, pp. 53-135). The polyclonal antibodies formed in the animal as a result of an immunological reaction can then be easily isolated from the blood according to generally known methods and purified, for example, by means of column chromatography. Monoclonal antibodies can be produced, for example, according to the known method of Winter & Milstein (Winter and Milstein, 1991, Nature 349:293-299).

The present invention further relates to an antibody or antibody fragments directed against a polypeptide described above and reacts specifically with the polypeptides described above, where the above-mentioned parts of the polypeptide are either immunogenic themselves or can be rendered immunogenic by coupling to suitable carriers, such as, for example, bovine serum albumin or keyhole limpet hemocyanin to increase in their immunogenicity. This antibody is either polyclonal or monoclonal; preferably it is a monoclonal antibody.

Still further, the present invention relates to the generation and/or production of an antibody or antibody fragment specific for the polypeptide according to the invention from a recombinant antibody expression library, such as for example described by Knappik et al. (2000, J. Molec. Biol. 296:57-86) or by Chadd and Chamow (2001 Curr. Opin. Biotechnol. 12:188-94).

In another embodiment of the invention, it is provided an array, wherein the array contains at least two compounds selected from the group consisting of a polypeptide according to the invention, a functional variant thereof, a nucleic acid encoding the polypeptide, a non-functional mutant variant the nucleic acid and an antibody or an antibody fragment directed against the polypeptide. Alternatively, the array may contain at least one component according to the invention in combination with previously described components implicated in neoplastic or metabolic liver disorders or epithelial cancers.

Within the meaning of the invention the term “array” refers to a solid-phase or gel-like carrier upon which at least two compounds are attached or bound in one-, two- or three-dimensional arrangement. Such arrays are generally known to the person skilled in the art and are typically generated on glass microscope slides, specially coated glass slides such as polycation-, nitrocellulose- or biotin-coated slides, cover slips, and membranes such as for example membranes based on nitrocellulose or nylon.

The aforementioned arrays include bound polypeptides according to the invention or functional variants thereof or nucleic acids coding for the polypeptides, or variants thereof, fusion proteins according to the invention or antibodies or antibody fragments directed against polypeptides according to the invention or functional variants thereof or cells expressing polypeptides according to the invention or functional variants thereof or at least two cells expressing at least one nucleic acid according to the invention, or variants thereof. Nucleic acids coding for these, or variants thereof can also be part of an array. Such arrays can be employed for analysis and/or diagnosis of liver disorders, preferably of HCC, and/or epithelial cancer.

The invention further relates to a method of producing arrays according to the invention, wherein at least two compounds according to the invention are bound to a carrier material.

Methods of producing such arrays, for example based on solid-phase chemistry and photo-labile protective groups are generally known (U.S. Pat. No. 5,744,305). Such arrays can also brought into contact with substances or a substance libraries and tested for interaction, for example for binding or change of conformation.

The invention further relates to a process for preparing an array immobilized on a support material for analysis and/or diagnosis of disorders according to the invention such as a liver disorder, preferably of HCC, in which at least two nucleic acid, at least two polypeptide or at least two antibody or antibody fragment, and/or at least two cell, or at least one of the aforementioned components in combination with other components relevant to neoplastic and metabolic liver disorders or epithelial cancers, as described above, is used for preparation. The arrays produced by such process can be employed for the diagnosis of disorders according to the invention.

In another aspect the invention relates to a diagnostic contains at least one compound selected from the group consisting of a polypeptide according to the invention, or a functional variant thereof, a nucleic acid encoding the polypeptide, preferably a nucleic acid according to SEQ ID 10 to 19, or a variant of one of the aformementioned nucleic acids, and an antibody or an antibody fragment according to the invention, combined or together with suitable additives or auxiliaries.

Compared to the state of the art, this diagnostic surprisingly allow improved, more sensitive, earlier, faster, and/or non-invasive diagnosis of liver disorders and/or other epithelial cancers.

Within the meaning of the invention “suitable additives” or “auxiliaries” are generally known to the person skilled in the art and comprise, for example, physiological saline solution, demineralized water, gelatin or glycerol-based protein stabilizing reagents. Alternatively, the nucleic acid or polypeptide according to the invention may be lyophilized for stabilization.

In another example a diagnostic kit based on the nucleic acid sequences according to the invention could be generated. Such a kit may be designed specifically to detect cells altered as a result of the described disorders resident in the circulatory system and thereby detectable in serum from test patients. Additional examples of diagnostic kits includes enzyme linked immunosorbent assays (ELISA), radioimmunoassays (RIA), and detection of an immune reaction or specific antibodies to the polypeptides according to the invention including detection of specific responding immune cells.

In a preferred embodiment the diagnostic according to the invention contains a probe, preferentially a DNA probe.

For example, it is possible according to the present invention to prepare a diagnostic based on the polymerase chain reaction (PCR). Under defined conditions, preferably using primers specific for a nucleic acid according to the invention as a DNA probe PCRs specific for the nucleic acid sequences of the invention will be utilized to monitor both the presence, and especially the amount, of specific nucleic acids according to the invention in a sample isolated from a patient obtained for diagnostic or therapeutic purposes. This opens up a further possibility of obtaining the described nucleic acids, for example by isolation from a suitable gene or cDNA library, for example from a liver disorder-specific or liver specific gene bank, with the aid of a suitable probe (see, for example, J. Sambrook et al., 1989, Molecular Cloning. A Laboratory Manual 2nd edn. Cold Spring Harbor Laboratory, Cold Spring Harbor, N.Y. Chapter 8 pages 8.1 to 8.81, Chapter 9 pages 9.47 to 9.58 and Chapter 10 pages 10.1 to 10.67).

Suitable probes are, for example, DNA or RNA fragments having a length of about 50-1000 nucleotides, preferably having a length of about 10 to about 100 nucleotides, preferably about 100 to about 200 nucleotides, in particular having a length of about 200-500 nucleotides, whose sequence can be derived from the polypeptides according to SEQ ID 1 to SEQ ID 9 and/or SEQ ID 47, and functional variants thereof, and nucleic acids coding for the polypeptides, preferably according to SEQ ID 10 to SEQ ID 19, and variants thereof.

Alternatively, it is preferably possible with the aid of the derived nucleic acid sequences to synthesize oligonucleotides that are suitable as primers for a polymerase chain reaction. Using this, the nucleic acid described above or parts of this can be amplified and isolated from cDNA, for example HCC-specific cDNA. Suitable primers are, for example, DNA fragments having a length of about 10 to 100 nucleotides, preferably having a length of about 15 to 50 nucleotides, in particular having a length of 17 to 30 nucleotides, whose sequence can be derived from the polypeptides according to SEQ ID 1 to SEQ ID 9 and/or SEQ ID 47 from the nucleic acids according to SEQ ID 10 to SEQ ID 19. The design and synthesis of such primers is generally known to the person skilled in the art. The primers may additionally contain restriction sites, e.g. suitable for integration of the amplified sequence into vectors, or other adapters or overhang sequences, e.g. having a marker molecule such as a fluorescent marker attached, generally known to the skilled worker.

In another aspect of the invention it is provided a method of diagnosis of a disorder according to the invention, wherein at least one compound selected from the group consisting of a polypeptide according to the sequence of SEQ ID 1 to SEQ ID 9 and/or SEQ ID 47, a functional variant thereof, a nucleic acid encoding the polypeptide, a variant of one of the aforementioned nucleic acids, and an antibody directed against the polypeptide or antibody fragment thereof, is identified in the sample of a patient and compared with at least one compound of a reference library or of a reference sample.

In a preferred embodiment of the method the disorder of the liver is a disorder selected from the group consisting of cirrhosis, alcoholic liver disease, chronic hepatitis, Wilson's disease, heamochromatosis, hepatocellular carcinoma, benign liver neoplasms, and focal nodular hyperplasia.

In a preferred embodiment of the method the epithelial cancer is an adenocarcinoma of any organ other than liver, preferably of an organ selected from the group consisting of the lung, the stomach, the kidney, the colon, the prostate, the skin, and the breast.

Compared to the state of the art, this diagnostic surprisingly allows improved, more sensitive, earlier, faster, and/or non-invasive diagnosis of the liver disorders and/or other epithelial cancers.

Preferably the sample is isolated from a patient by non-invasive methods as described above.

For example, serum detection of specific deregulated gene proteins via ELISA assay is one application, alternatively one or a panel of antibodies to deregulated gene products from which a diagnostic score is deduced based on the combinations of, and expression levels of gene products expressed in the diseased tissue or in serum from diseased individuals.

A preferred diagnostic according to the invention contains the described polypeptide or the immunogenic parts thereof described in greater detail above. The polypeptide or the parts thereof, which are preferably bound to a solid phase, e.g. of nitrocellulose or nylon, can be brought into contact in vitro, for example, with the body fluid to be investigated, e.g. blood, serum, plasma, ascitic fluid, pleural effusion, cerebral spinal fluid, saliva, urine, semen, in order thus to be able to react, for example, with autoimmune antibodies present in e.g. the blood of the patient. The antibody-peptide complex can then be detected, for example, with the aid of labeled antihuman IgG antibodies. The labeling involves, for example, an enzyme, such as peroxidase, which catalyses a color or chemiluminescent reaction. The presence and the amount of autoimmune antibody present can thus be detected easily and rapidly by means of the color.

In addition the diagnostic may be directed to detecting an endogenous antibody or fragment thereof present in the sample isolated from a patient which antibody or fragment thereof is directed against a polypeptide according to the invention. Detection of such autoimmune antibodies may be accomplished by methods generally known to the skilled artisan, e.g. by immunoaffinity assays using polypeptides according to the invention or functional variants thereof or parts thereof as a probe. Preferably the presence of such autoimmune antibodies is indicative of the patient suffering from a disorder according to the invention.

A further diagnostic, that is subject matter of the present invention, contains the antibodies according to the invention themselves. With the aid of these antibodies, it is possible, for example, to easily and rapidly investigate a tissue sample as to whether the concerned polypeptide according to the invention is present in an increased amount in order to thereby obtain an indication of possible disease including liver disorders, for example HCC. In this case, the antibodies according to the invention are preferably labeled directly, or more commonly for example these are detected with a specific secondary antibody indirectly, such as with an enzyme or fluorescent molecule, as already described above. The specific antibody-peptide complex can thereby be detected easily and rapidly, e.g., by means of an enzymatic color reaction.

In still another aspect of the invention it is provided a method for identifying at least one nucleic acid according to SEQ ID 10 to SEQ ID 19, or a variant thereof differentially expressed in a sample isolated from a patient relative to a reference library or a reference sample comprising the following steps:
(a) detecting the expression of at least one nucleic acid according to SEQ ID 10 to SEQ ID 19, or a variant thereof in a sample isolated from a patient,
(b) comparing the expression of said nucleic acid(s) detected in step (a) with the expression of the same nucleic acid(s) in a reference library or in a reference sample,
(c) identifying said nucleic acid(s) which is (are) differentially expressed in the sample isolated from the patient compared to the reference library or the reference sample.

Compared to the state of the art the method surprisingly allows improved, more sensitive, earlier, faster, and/or non-invasive identification of differentially expressed nucleic acids according to the invention that provides a useful basis for diagnosing a disorder according to the invention.

Preferably at least 2, at least 3, at least 4 at least 5, at least 6, or at least 7 nucleic acids are identified.

In another preferred embodiment of the method said nucleic acid(s) is (are) detected by PCR based detection or by a hybridization assay.

In another preferred embodiment of the method the expression of said nucleic acid is compared by a method selected from the group consisting of solid-phase based screening methods, hybridization, subtractive hybridization, differential display, and RNase protection assay.

In a further preferred embodiment of the method the sample isolated from the patient is selected from the group consisting of liver tissue, a liver cell, tissue from another organ subject to cancerous transformation, a cell from this organ, blood, serum, plasma, ascitic fluid, pleural effusion, cerebral spinal fluid, saliva, urine, semen, and feces.

Preferably the reference sample is isolated from a source selected from a non-diseased sample of the same patient or a non-diseased sample from another subject. The selection of appropriate reference samples is generally known to the person skilled in the art. In particular the reference sample may be selected from the group consisting of liver tissue, a liver cell, blood, serum, plasma, ascitic fluid, pleural effusion, cerebral spinal fluid, saliva, urine, semen, and feces.

In another preferred embodiment of the method, the reference library is an expression library or a data base comprising clones or data on non-diseased expression of at least one nucleic acid according to the invention in samples that preferably may be selected from the group consisting of liver tissue, a liver cell, blood, serum, plasma, ascitic fluid, pleural effusion, cerebral spinal fluid, saliva, urine, semen, and feces.

In another aspect of the invention it is provided a method of diagnosing a liver disorder, and/or another epithelial cancer comprising the following steps:
(a) detecting the expression of at least one nucleic acid according to SEQ ID 10 to SEQ ID 19 and/or SEQ ID 47, or a variant thereof in a sample isolated from a patient,
(b) comparing the expression of said nucleic acid(s) detected in step (a) with the expression of the same nucleic acid(s) in a reference library or in a reference sample,
(c) identifying said nucleic acid which is differentially expressed in the sample isolated from the patient compared to the reference library or the reference sample, and
(d) matching said nucleic acid(s) identified in step (c) with said nucleic acid(s) differentially expressed in a pathologic reference sample or pathologic reference library,
   wherein the matched nucleic acid(s) is (are) indicative of the patient suffering from a liver disorder, and/or other epithelial cancer.

Compared to the state of the art, this method of diagnosing surprisingly allows improved, more sensitive, earlier, faster, and/or non-invasive diagnosis of the liver disorders and/or other epithelial cancers.

Preferably at least 2, at least 3, at least 4 at least 5, at least 6, or at least 7 nucleic acids are identified.

In another preferred embodiment of the method said nucleic acid(s) is (are) detected by PCR based detection or by a hybridization assay.

In another preferred embodiment of the method the expression of the said nucleic acid is compared by a method selected from the group consisting of solid-phase based screening methods, hybridization, subtractive hybridization, differential display, and RNase protection assay.

In a further preferred embodiment of the method the sample isolated from the patient is selected from the group consisting of liver tissue, a liver cell, tissue from another organ subject to cancerous transformation, a cell from this organ, blood, serum, plasma, ascitic fluid, pleural effusion, cerebral spinal fluid, saliva, urine, semen, and feces.

Preferably the reference sample is isolated from a source selected from a non-diseased sample of the same patient or a non-diseased sample from another subject. The selection of appropriate reference samples is generally known to the person skilled in the art. In particular the reference sample may be selected from the group consisting of liver tissue, a liver cell, blood, serum, plasma, ascitic fluid, pleural effusion, cerebral spinal fluid, saliva, urine, semen, and feces.

In another preferred embodiment of the method of diagnosis, the reference library is an expression library or a data base comprising clones or data on non-diseased expression of said nucleic acid(s) according to the invention in samples that preferably may be selected from the group consisting of liver tissue, a liver cell, blood, serum, plasma, ascitic fluid, pleural effusion, cerebral spinal fluid, saliva, urine, semen, and feces.

In another preferred embodiment of the method of diagnosis, the pathologic reference sample is isolated from a diseased sample from another patient. The latter patient having been diagnosed as suffering from the disorder according to the invention which is to be diagnosed. The selection of appropriate pathologic reference samples is generally known to the person skilled in the art. In particular the pathologic reference sample may be selected from the group consisting of liver tissue, a liver cell, blood, serum, plasma, ascitic fluid, pleural effusion, cerebral spinal fluid, saliva, urine, semen, and feces.

In another preferred embodiment of the method of diagnosis, the pathologic reference library is a data base comprising data on differential expression of the at least one nucleic acid according to the invention in samples isolated from at least one patient, excluding the patient under diagnosis, suffering from the disorder according to the invention to be diagnosed in the inventive method relative to control expression in a reference sample or reference library. The pathologic reference library preferably also relates to a differential expression library comprising nucleic acids according to the invention which are differentially expressed in samples isolated from at least one patient, excluding the patient under diagnosis, suffering from the disorder according to the invention to be diagnosed in the inventive method relative to control expression in a reference sample or reference library. The selection of an appropriate pathologic reference library is generally known to the person skilled in the art.

Preferably the liver disorder is a disorder selected from the group consisting of cirrhosis, alcoholic liver disease, chronic hepatitis, Wilson's Disease, heamochromatosis, hepatocellular carcinoma, benign liver neoplasms, and focal nodular hyperplasia. In particular the epithelial cancer is an adenocarcinoma of any organ other than liver, preferably of an organ selected from the group consisting of the lung, the stomach, the kidney, the colon, the prostate, the skin, and the breast.

Within the meaning of the invention the term “detecting a nucleic acid” refers to a method that preferably uncovers, visualizes, separates or allows recognition of the nucleic acid according to the invention from the background of the other components present in the sample. Such methods are generally known to the person skilled in the art and include in situ hybridization, PCR amplification, gel electrophoresis, northern blots, solid phase array (gene chips) based methods, nuclease protection methods (as described and referenced in Alberts, et al. (2002) The Molecular Biology of the Cell, 4^{th} ed. Garland, N.Y., USA).

Within the meaning of the invention the term “comparing the expression of said nucleic acid(s) detected in step (a) with the expression of the same nucleic acid(s) in a reference library or in a reference sample” refers to a comparison of the expression of the two groups of said nucleic acid(s) on a quantitative or qualitative level by means of an experimental procedure such as differential display, subtractive hybridization, RNAse protection assay, or especially DNA chip hybridization. Moreover a comparison of experimental data on said nucleic acid(s) detected in step (a) with the expression of the same nucleic acid(s) in a reference library as defined above is also included herein.

The term “identifying said nucleic acid(s) which is (are) differentially expressed in the sample isolated from the patient compared to the reference library or the reference sample” within the meaning of the present invention is understood to mean selecting said nucleic acid(s) which is (are) differentially expressed compared to the reference library or the reference samples which fulfills the following criteria: the level of differential expression of the detected said nucleic acid(s) compared to the reference library or the reference samples is greater than about 2 fold, preferably greater than about 5 fold, more preferred greater than about 10 fold upregulated.

The term “matching said nucleic acid(s) identified in step (c) with said nucleic acid(s) differentially expressed in a pathologic reference sample or pathologic reference library” within the meaning of the invention is understood to mean that said nucleic acid(s) identified in step (c) is (are) compared with said nucleic acid(s) differentially expressed in a pathologic reference sample or pathologic reference library. Then said nucleic acid(s) identified in step (c) that is (are) also differentially expressed in the pathologic reference sample or pathologic reference library is (are) matched, i.e. said identical pair is identified and allocated. Since the differential expression of said nucleic acid(s) in the pathologic reference sample or pathologic reference library is (are) indicative of a disorder according to the invention, such correspondence with the differential expression in the sample then indicates that the patient suffers from that disorder.

Preferably the sample is isolated from a patient by non-invasive or preferably minimally invasive methods such as described above, including venupuncture.

The methods of diagnosing according to the invention allows early detection of a liver disorder and/or an epitherlial cancer, and/or non-invasive diagnosis of the disorder, based on an essentially concordant expression pattern of the nucleic acids according to the invention detected in the samples isolated from an animal and/or a human patient suffering from a liver disorder and/or an epithelial cancer relative to a reference sample or relative to a reference library. The method has the additional advantage that it also provides additional and novel diagnostic parameters to characterize different subtypes of liver disorders, such as for example subtypes of HCC.

The term “essentially concordant expression pattern” of the nucleic acids according to the invention refers to a pattern of expression that is essentially reproducible from patient to patient or subject to subject, provided that the patients or subjects compared are in the same or comparable pathological condition or healthy condition, respectively.

In still another aspect of the invention it is provided a method for identifying at least one polypeptide according to SEQ ID 1 to SEQ ID 9 and/or SEQ No. 47, or a functional variant thereof differentially expressed in a sample isolated from a patient relative to a reference library or a reference sample comprising the following steps:
(a) detecting the expression of at least one polypeptide according to SEQ ID 1 to SEQ ID 9 and/or SEQ ID 47, or a functional variant thereof in a sample isolated from a patient,
(b) comparing the expression of said polypeptide(s) detected in step (a) with the expression of said polypeptide(s) in a reference library or in a reference sample,
(c) identifying said polypeptide(s) which is (are) differentially expressed in the sample isolated from the patient compared to the reference library or the reference sample.

Compared to the state of the art, this method surprisingly allows improved, more sensitive, earlier, faster, and/or non-invasive identification of differentially expressed polypeptides according to the invention that provides a useful basis for diagnosing a disorder according to the invention.

Preferably at least 2, at least 3, at least 4, at least 5, at least 6, or at least 7 polypeptides are identified.

Preferably the sample is isolated from a patient by non-invasive or minimally invasive methods such as described above, including venupuncture.

In another embodiment of the method the sample is a sample as defined further above. Preferably the reference sample is a reference sample as defined above.

In another preferred embodiment of the method, the reference library is an expression library or a data base comprising clones or data on non-diseased expression of the at least one polypeptide according to the invention in samples that preferably may be selected from the group consisting of liver tissue, a liver cell, blood, serum, plasma, ascitic fluid, pleural effusion, cerebral spinal fluid, saliva, urine, semen, or feces. Such databases are generated as a result of the cDNA microarray expression analysis according to the invention and are known to persons skilled in the art. Further reference libraries useable according to the invention have been described above.

In another aspect of the invention it is provided a method of diagnosing a liver disorder and/or an epithelial cancer comprising the following steps:
(a) detecting the expression of at least one polypeptide according to SEQ ID 1 to SEQ ID 9 and/or SEQ ID 47, and/or a functional variant thereof in a sample isolated from a patient,
(b) comparing the expression of said polypeptide(s) detected in step (a) with the expression of said polypeptide(s) in a reference library or in a reference sample,
(c) identifying said polypeptide(s) which is (are) differentially expressed in the sample isolated from the patient compared to the reference library or the reference sample, and
(d) matching said polypeptide(s) identified in step (c) with said polypeptide(s) differentially expressed in a pathologic reference sample or pathologic reference library,
   wherein the matched polypeptide(s) is (are) indicative of the patient suffering from a liver disorder and/or an epithelial cancer.

Compared to the state of the art, this method of diagnosing surprisingly allows improved, more sensitive, earlier, faster, and/or non-invasive diagnosis of the liver disorders and/or other epithelial cancers.

Preferably at least 2, at least 3, at least 4, at least 5, at least 6, or at least 7 polypeptides are identified.

Within the meaning of the invention the term “detecting a polypeptide” refers to a method that preferably uncovers, visualizes, separates and/or allows recognition of the polypeptide according to the invention from the background of the other components present in the sample. Such methods are generally known to the person skilled in the art and includes gel electrophoresis, chromatographic techniques, immunoblot analysis, immunohistochemistry, enzyme based immunoassay, mass spectroscopy, high pressure liquid chromatography, surface plasmon resonance, and/or antibody and protein arrays as described above (Ausubel, F. A. et al., eds., 1990, Current Protocols in Molecular Biology. Greene Publishing and Wiley-Interscience, New York, USA, Chapter 10; Myszka and Rich 2000, Pharm. Sci. Technol. Today 3:310-317). Preferably proteins and polypeptides are prepared from the sample by disruption of the cells with physical sheering or ultrasonic means, for example. Protein is denatured and stabilized with reducing agent treatment and heating and the protein is size fractionated on electrophoretic polyacrylamide gels.

Within the meaning of the invention the term “comparing the expression of said polypeptide(s) detected in step (a) with the expression of the same polypeptide(s) in a reference library or in a reference sample” refers to a comparison of the expression of the two groups of polypeptide(s) on a quantitative and/or qualitative level by means of an experimental procedure such as two dimensional gel electrophoresis, chromatographic separation techniques, immunoblot analysis, surface plasmon resonance, immunohistochemistry, and enzyme based immunoassay. In two dimensional gel electrophoresis, all peptides are first resolved according to isoelectric point in the first electrophoretic dimension and then by size according to methods well known to persons experienced in the art. Moreover a comparison of experimental data on the at least one polypeptide detected in step 1 with the expression of the polypeptide in a reference library as defined above is also included herein.

The term “Identifying said polypeptide(s) which is (are) differentially expressed in the sample isolated from the patient compared to the reference library or the reference sample” within the meaning of the present invention is understood to mean selecting said polypeptide(s) which is (are) differentially expressed compared to the reference library or the reference samples which fulfills the following criteria: the level of differential expression of the detected polypeptide(s) compared to the reference library or the reference samples is greater than about 2 fold, preferably greater than about 5 fold, more preferred greater than about 10 fold upregulated.

The term “matching said polypeptide(s) identified in step (c) with said polypeptide(s) differentially expressed in a pathologic reference sample or pathologic reference library” within the meaning of the invention is understood to mean that said polypeptide(s) identified in step (c) is compared with said polypeptide(s) differentially expressed in a pathologic reference sample or pathologic reference library. Then said polypeptide(s) identified in step (c) that is (are) also differentially expressed in the pathologic reference sample or pathologic reference library is (are) matched, i.e. said identical pair(s) is (are) identified and allocated. Since the differential expression of said polypeptide(s) in the pathologic reference sample or pathologic reference library is (are) indicative of a disorder according to the invention, such correspondence with the differential expression in the sample then indicates that the patient suffers from that disorder.

Preferably the sample is isolated from a patient by non-invasive or minimally invasive methods such as described above, including venupuncture.

In another embodiment of the method the sample is a sample as defined further above. Preferably the reference sample is a reference sample as defined above.

In another preferred embodiment of the method of diagnosis, the reference library is an expression library or a dataset comprising clones or data on non-diseased expression of the at least one polypeptide according to the invention in samples that preferably may be selected from the group consisting of liver tissue, a liver cell, blood, serum, plasma, ascitic fluid, pleural effusion, cerebral spinal fluid, saliva, urine, semen, and feces.

An example of a data base according to the invention and further experimental reference libraries useable according to the invention have been described above.

In another preferred embodiment of the method of diagnosis, the pathologic reference sample is a pathologic reference sample as has been defined above.

In another preferred embodiment of the method of diagnosis, the pathologic reference library is a data base comprising data on differential expression of said polypeptide(s) according to the invention in samples isolated from at least one patient, excluding the patient under diagnosis, suffering from the disorder according to the invention to be diagnosed in the inventive method relative to control expression in a reference sample or reference library. The pathologic reference library also relates to a differential expression library comprising polypeptides according to the invention which are differentially expressed in samples isolated from at least one patient, excluding the patient under diagnosis, suffering from the disorder according to the invention to be diagnosed in the inventive method relative to control expression in a reference sample or reference library. The selection of an appropriate pathologic reference library is generally known to the person skilled in the art.

Preferably the liver disorder is a disorder selected from the group consisting of cirrhosis, alcoholic liver disease, chronic hepatitis, Wilson's Disease, heamochromatosis, hepatocellular carcinoma, benign liver neoplasms, and focal nodular hyperplasia. In particular the epithelial cancer is an adenocarcinoma of any organ other than liver, preferably of an organ selected from the group consisting of the lung, the stomach, the kidney, the colon, the prostate, the skin, and the breast.

The methods of diagnosing according to the invention allows early detection of a liver disorder and/or epithelial cancer, and/or non-invasive diagnosis of the disorder, based on an essentially concordant expression pattern of the polypeptides according to the invention detected in the samples isolated from an animal and/or a human patient suffering from a liver disorder and/or epithelial cancer relative to a reference sample or relative to a reference library. The method has the additional advantage that it also provides additional and novel diagnostic parameters to characterize different subtypes of liver disorders, such as for example subtypes of HCC.

The term “essentially concordant expression pattern” of the polypeptides according to the invention refers to a pattern of expression that is essentially reproducible from patient to patient or subject to subject, provided that the patients or subjects compared are in the same or comparable pathological condition or healthy condition, respectively.

In another aspect of the invention it is provided a pharmaceutical composition comprisinging at least one compound selected from the group consisting of a polypeptide according to the invention, a functional variant thereof, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of the antibody directed against one of the aforementioned polypeptides, a vector comprising a nucleic acid coding for the aforementioned antibody, a cell comprising the vector comprising a nucleic acid coding for the aforementioned antibody, and a cell comprising the vector comprising a nucleic acid coding for the aforementioned antibody fragment, combined or together with suitable additives or auxiliaries. In a preferred embodiment the pharmaceutical composition contains at least one cell according to the invention, combined or mixed together with suitable additives or auxiliaries.

When compared to the state of the art of therapy of liver disorders, and/or other epithelial cancers the pharmaceutical composition according to the invention surprisingly provide an improved, sustained and/or more effective treatment.

A pharmaceutical composition in the sense of the invention encompasses medicaments which can be used for preventing and/or treating a liver disorders and/or epithelial cancer. The pharmaceutical composition includes, for instance, a stabilized recombinant antibody that has been produced by expression of specific antibody gene fragments in a cellular system, preferably a eukaryotic system. A recombinant antibody therapeutic for instance, is delivered by injection into the diseased liver region or into the venous or arterial vascular systems or into the hepatic portal system. The injections can be repeated at regular intervals to achieve therapeutic efficacy. Therapeutics according this invention may also be employed in combinations with other chemical, antibody, or any other therapeutic application to improve efficacy.

The present invention also relates to a process producing a pharmaceutical composition for the treatment and/or prevention of disorders according to the invention, for example, HCC, in which at least one component selected from the group consisting of a polypeptide according to the invention, a functional variant thereof, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of the antibody directed against one of the aforementioned polypeptides, a vector comprising a nucleic acid coding for one of the aforementioned antibodies, a cell comprising the vector comprising a nucleic acid coding for one of the aforementioned antibodies, and a cell comprising the vector comprising a nucleic acid coding for one of the aforementioned antibody fragments, is combined or mixed together with suitable additives.

The present invention furthermore relates to a pharmaceutical composition produced by this process for the treatment and/or prevention of liver disorders and/or epithelial cancers, for example, HCC, which contains at least one component selected from the group consisting of a polypeptide according to the invention, a functional variant thereof, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of the antibody directed against one of the aforementioned polypeptides, a vector comprising a nucleic acid coding for one of the aforementioned antibodies, a cell comprising the vector comprising a nucleic acid coding for one of the aforementioned antibodies, and a cell comprising the vector comprising a nucleic acid coding for one of the aforementioned antibody fragments, if appropriate together with suitable additives and auxiliaries. The invention furthermore relates to the use of this pharmaceutical composition for the prevention and/or treatment of liver disorders, for example, HCC and/or epithelial cancer.

Preferably the pharmaceutical composition is employed for the treatment of a liver disorder selected from the group consisting of cirrhosis, alcoholic liver disease, chronic hepatitis, Wilson's Disease, heamochromatosis, hepatocellular carcinoma, benign liver neoplasms, and focal nodular hyperplasia. In particular the phramaceutical composition is employed for the treatment of an epithelial cancer that is an adenocarcinoma of any organ other than liver, preferably of an organ selected from the group consisting of the lung, the stomach, the kidney, the colon, the prostate, the skin, and the breast.

Therapy can also be carried out in a conventional manner generally known to the person skilled in the art, e.g. by means of oral application or via intravenous injection of the pharmaceutical compositions according to the invention. It is thus possible to administer the pharmaceutical composition comprising the suitable additives or auxiliaries, such as, for example, physiological saline solution, demineralized water, stabilizers, proteinase inhibitors.

A therapy based on the use of cells, which express at least one polypeptide according to the invention, functional variants thereof or nucleic acids coding for the polypeptide, or variants thereof can be achieved by using autologous or heterologous cells. Preferred cells comprise liver cells, for example primary cultures of liver cells, liver populating stem or progenitor cells, or blood cells. The cells can be applied to the tissue, preferably to the blood or injected into the liver, with suitable carrier material. Such therapy is preferably based on the notion that upon expression and/or release of a polypeptide according to the invention the polypeptide stimulates an immune response in the patient in need of the treatment.

Preferably the therapeutical approach is directed toward inhibiting the function and/or expression of at least one polypeptide according to the invention and/or the function and/or expression of at least one nucleic acid according to the invention. Such inhibition of the expression and/or function preferably reduces the expression and/or function of the targeted nucleic acid/polypeptide significantly. The inhibition of the expression and/or function preferably abolishes the expression and/or functioning of the targeted nucleic acid/polypeptide. Such reduction or abolished expression and/or functioning of the targeted nucleic acid/polypeptide can be determined using conventional assays for determining the expression and/or functioning of a polypeptide/nucleic acid generally known to the person skilled in the art. In particular such assays for determining the function comprise methods for comparing the biological activity of the targeted nucleic acid/polypeptide before and after administration of the pharmaceutical composition. Preferably such assays for determining the expression comprise methods for comparing the level of expression of the targeted nucleic acid/polypeptide before and after administration of the pharmaceutical composition.

Such therapy is preferably accomplished by the use of a nucleic acid having a sequence complementary to one of nucleic acids according to the invention, i.e. an antisense molecule or a RNA interference molecule which reduces or abolishes the translation of transcribed nucleic acids according to the invention and thereby inhibits the function and/or expression of the targeted nucleic acid/polypeptide. Preferably such nucleic acid having a complementary sequence may be employed in the form of a vector or a cell comprising such nucleic acid. On the polypeptide level the therapy may in particular be carried out by the use of an antibody or an antibody fragment directed against a polypeptide according to the invention. The antibody or antibody fragment may be administered directly to the patient or preferably the nucleic acid encoding the antibody is contained in a vector which is preferably contained in a cell. The cell or vector may then be administered to the patient in need of such treatment.

When compared to the state of the art of therapy of liver disorders, and/or other epithelial cancers the method of treating according to the invention surprisingly provide an improved, sustained and/or more effective treatment.

The invention further relates to a method of treating a patient suffering from of a liver disorder, wherein at least one component selected from the group consisting of a polypeptide according to the invention, a functional variant thereof, a nucleic acid encoding the polypeptide, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the vector, an antibody directed against the polypeptide, a fragment of the antibody, a vector comprising a nucleic acid coding for the antibody, a cell comprising the vector comprising a nucleic acid coding for the antibody, and a cell comprising the vector comprising a nucleic acid coding for the antibody fragment, optionally combined or together with suitable additives and/or auxilaries, is administered to the patient in need of a the treatment in a therapeutically effective amount.

Preferably the method of treatment is directed to a liver disorder selected from the group consisting of cirrhosis, alcoholic liver disease, chronic hepatitis, Wilson's disease, heamochromatosis, hepatocellular carcinoma, benign liver neoplasms, and focal nodular hyperplasia. In particular the method of treatment is directed to an epithelial cancer that is an adenocarcinoma of any organ other than liver, preferably of an organ selected from the group consisting of the lung, the stomach, the kidney, the colon, the prostate, the skin, and the breast.

Methods of administering such compounds or cells have been described in detail above.

The term “therapeutically effective amount” refers to the administration of an amount of the compound to the patient that results in an “effective treatment” as defined above. Determination of the therapeutically effective amount of the compound(s) is generally known to the person skilled in the art.

Such methods of treating allow effective treatment of a liver disorder and/or epithelial cancers as described above.

In another aspect of the invention it is provided a method of stimulating an immune response a patient suffering from a liver disorder and/or an epithelial cancer to a polypeptide according to the invention, or a functional variant thereof, wherein at least one component selected from the group consisting of a polypeptide according to the invention, a functional variant thereof, a nucleic acid encoding one of the aforementioned polypeptides, a variant of one of the aforementioned nucleic acids, a vector comprising one of the aforementioned nucleic acids, a cell comprising one of the aforementioned nucleic acids, and a cell comprising the aforementioned vector, is administered to the patient in need of such treatment in an amount effective to stimulate the immune response in the patient.

When compared to the state of the art of therapy of liver disorders, and/or other epithelial cancers the method of stimulating an immune response according to the invention surprisingly provide an improved, sustained and/or more effective immunization.

In another aspect of the invention it is provided a method of preventing a patient from developing a liver disorder and/or an epithelial cancer, wherein at least one component selected from the group consisting of a polypeptide according to the invention, a functional variant thereof, a nucleic acid encoding one of the aforementioned polypeptides, a variant of one of the aforementioned nucleic acids, a nucleic acid having a sequence complementary to one of the aforementioned nucleic acids, a nucleic acid which is a non-functional mutant variant of one of the aforementioned nucleic acids, a vector comprising one of the aforementioned nucleic acids, a cell comprising one of the aforementioned nucleic acids, and a cell comprising the aforementioned vector, is administered to the patient in need of such preventive treatment in a therapeutically effective amount.

When compared to the state of the art of therapy of liver disorders, and/or other epithelial cancers the method of preventing according to the invention surprisingly provide an improved, sustained and/or more effective preventive measure.

Preferably the method of preventing and/or method of stimulating an immune response is directed to a liver disorder selected from the group consisting of cirrhosis, alcoholic liver disease, chronic hepatitis, Wilson's Disease, heamochromatosis, hepatocellular carcinoma, benign liver neoplasms, and focal nodular hyperplasia. In particular, preferably the method of preventing and/or method of stimulating an immune response is directed to an epithelial cancer which is an adenocarcinoma of any organ other than liver, preferably of an organ selected from the group consisting of the lung, the stomach, the kidney, the colon, the prostate, the skin, and the breast.

In a further aspect the invention relates to a method of identifying at least one pharmacologically active compound comprising the following steps:
(a) providing at least one polypeptide according to the SEQ ID 1 to 9 and/or SEQ ID 47, or a functional variant thereof,
(b) contacting said polypeptide(s), with suspected to be pharmacologically active compound(s),
(c) assaying the interaction of said polypeptide(s) of step (a) with said compund(s) suspected to be pharmacologically active,
(d) identifying said compound (s) suspected to be pharamacologicaly active which directly or indirectly interact with said polypeptide(s) of step (a).

Preferably said polypeptide(s) is (are) provided in a form selected from the group of said polypeptide(s) is (are) attached to a column, said polypeptide(s) is (are) attached to an array, said polypeptide(s) is (are) contained in an electrophoresis gel, said polypeptide is attached to a membrane, and said polypeptide(s) is (are) expressed by a cell.

It is preferred to assay the interaction by a method selected from the group of enzyme and fluorescence based cellular reporter assays in which interaction of the compound suspected to be pharmacological active with a recombinant fusion protein including said polypeptide(s) of step (a) is detected. The interaction may preferably also be assayed by surface plasmon resonance, HPLC and mass spectroscopy. Preferably the direct or indirect interaction is selected from the group consisting of induction of the expression of said polypeptide(s), inhibition of the expression of said polypeptide(s), activation of the function of said polypeptide(s), inhibition of the function of said polypeptide(s).

The term “pharmacologically active substance” in the sense of the present invention is understood as meaning all those molecules, compounds and/or compositions and substance mixtures which can interact under suitable conditions with a polypeptide according to the SEQ ID 1 to 9 and/or SEQ ID 47, or functional variants thereof (encoded according to SEQ ID 10 to 19), if appropriate together with suitable additives and/or auxiliaries. Possible pharmacologically active substances are simple chemical (organic or inorganic) molecules or compounds, but can also include peptides, proteins or complexes thereof. Examples of pharmacologically active substances are organic molecules that are derived from libraries of compounds that have been analyzed for their pharmacological activity. On account of their interaction, the pharmacologically active substances can influence the expression and/or function(s) of the polypeptide in vivo or in vitro or alternatively only bind to the polypeptides described above or enter into other interactions of covalent or non-covalent manner with them.

A suitable test system that can be used in accordance with the invention is based on identifying interactions with the two hybrid system (Fields and Stemglanz, 1994, Trends in Genetics, 10, 286-292; Colas and Brent, 1998 TIBTECH, 16, 355-363). In this test system, cells are transformed with expression vectors that express fusion proteins that consist of at least one polypeptide according to the invention and a DNA-binding domain of a transcription factor such as Gal4 or LexA. The transformed cells also contain a reporter gene whose promoter contains binding sites for the corresponding DNA-binding domain. By means of transforming a further expression vector, which expresses a second fusion protein consisting of a known or unknown polypeptide and an activation domain, for example from Gal4 or herpes simplex virus VP16, the expression of the reporter gene can be greatly increased if the second fusion protein interacts with the investigated polypeptide according to the invention. This increase in expression can be used for identifying new interacting partners, for example by preparing a cDNA library from e.g., liver tissue, or diseased liver tissue for the purpose of constructing the second fusion protein. In a preferred embodiment, the interaction partner is an inhibitor of a polypeptide according to the SEQ ID 1 to 9 and/or SEQ ID 47 (encoded by SEQ ID 10 to 19) or functional variants thereof. This test system can also be used for screening substances that inhibit an interaction between the polypeptide according to the invention and an interacting partner. Such substances decrease the expression of the reporter gene in cells that are expressing fusion proteins of the polypeptide according to the invention and the interacting partner (Vidal and Endoh, 1999, Trends in Biotechnology, 17: 374-81). In this way, it is possible to rapidly identify novel active compounds that can be employed for the therapy of and/or prevention of liver disorders and/or epithelial cancer.

Assays for identifying pharmacologically active substances that exert an influence on the expression of proteins are well known to the skilled person (see, for example, Sivaraja et al., 2001, U.S. Pat. No. 6,183,956). Thus, cells that express a polypeptide according to the SEQ ID 2 or functional variants thereof can be cultured as a test system for analyzing gene expression in vitro, with preference being given to liver cells. Gene expression is analyzed, for example, at the level of the mRNA or of the proteins using methods generally known to the person skilled in the art. In this connection, the quantity of a polypeptide according to the SEQ ID 1 to 9 and/or SEQ ID 47 (encoded by SEQ ID 10 to 19) or mRNA present after adding one or more putative pharmacologically active substances to the cell culture is measured and compared with the corresponding quantity in a control culture. This is done, for example, with the aid of an antibody specifically directed against the polypeptide according to the SEQ ID 1 to 9 and/or SEQ ID 47 (encoded by SEQ ID 10 to 19), or a functional variant thereof, which can be used to detect the polypeptide present in the lysate of the cells. The amount of expressed polypeptide can be quantified by methods generally known to the person skilled in the art using, for example, an ELISA or a Western blot. In this connection, it is possible to carry out the analysis as a high-throughput method and to analyze a very large number of substances for their suitability as modulators of the expression of a polypeptide according to SEQ ID 1 to 9 and/or SEQ ID 47 (encoded by SEQ ID 10 to 19) (Sivaraja et al., 2001, U.S. Pat. No. 6,183,956). In this connection, the substances to be analyzed can be taken from substance libraries (see, e.g. DE19816414, DE19619373) that can contain many thousands of substances, which are frequently very heterogeneous.

The invention will now be further illustrated below with the aid of the figures and examples, representing preferred embodiments and features of the invention without the invention being restricted hereto.

### BRIEF DESCRIPTION OF FIGURES

FIG. 1: RNA expression levels in HCC

Summary boxplot of expression values in HCC versus non-diseased liver cDNA microarray experiments is provided. The box plot is a graphical representation of log2 expression value ratios with the median value indicated by a horizontal line in each box. The extent of each box indicates the iqr; whiskers indicate of 1.5 times the iqr. Ratios that do not fall within this range are indicated with small circles. For each nucleic acid according to the invention, elevated expression is apparent in HCC. Expression values are consistently elevated in a similar ratio except for OBcl5 (SEQ ID 11) where the differences in expression between patient and control samples are most significant.

FIG. 2: Expression specificity of OBcl5 in HCC when compared to normal tissue(s) and other types of cancer

The quantity of OBcl5 specific PCR product is monitored by incorporation and hydrolysis of the Taqman fluorescently labeled gene specific probe using the primers OBcl5-p8, SEQ ID 66; OBcl5-p9, SEQ ID 67; and OBcl5-p10, SEQ ID 68. The quantitative assessment of expression of the OBcl5 (SEQ ID 11) by quantitative RT-PCR (Q-PCR) in HCC=A, FNH=B is compared to expression pattern in normal tissue (C=non-neoplastic (normal) Liver; D=Lung normal; F=Colon normal; H=Testis normal; J=Muscle normal; K=Skin normal; L=Heart normal; M=Kidney normal) and other cancers (E=Lung cancer; G=Colon cancer; I=Testis cancer). Mann-Whitney-U Test (non-parametric test applied for non-normally distributed data) is performed as Wilcoxon-Test with option paired=“false”, provides the sum of the ranks for the larger of the two groups (HCC) (=Wilcoxon value, W) and shows the significant differences (P-values) in OBcl5 distribution in all the tissues samples as illustrated in Table 7. (HCC=Hepatocellular Carcinoma; FNH=Focal Nodular Hyperplasia.; NNL=non-neoplastic (normal) Liver; Lung N=Lung normal; Col N=Colon normal; Tst. N=Testis normal; Ms. N=Muscle normal; Skin N=Skin normal; Hrt. N=Heart normal; Kdny. N=Kidney normal) and other cancers (Lung C=Lung cancer; Col. C=Colon cancer; Tst. C=Testis cancer).

TABLE 7
Distribution of Obcl5 in various tissue samples

| Data | W | P-value |
|---|---|---|
| HCC vs FNH | 71 | 0.0005468 |
| HCC vs NNL | 54 | 0.001504 |
| HCC vs Lung N | 54 | 0.001504 |
| HCC vs Lung C | 36 | 0.01053 |
| HCC vs Col. N | 54 | 0.001504 |
| HCC vs Col. C | 54 | 0.001504 |
| HCC vs Tst. N | 72 | 0.0002734 |
| HCC vs Tst. C | 54 | 0.001504 |
| HCC vs Ms. N | 72 | 0.0002734 |
| HCC vs Skin N | 54 | 0.001504 |
| HCC vs Hrt. N | 54 | 0.001504 |
| HCC vs Kdny. N | 54 | 0.001504 |

FIG. 3: RT-PCR data demonstrating expression of the nucleic acids (SEQ ID 10 to 19) in independent HCC samples and controls

Amplification of the ‘housekeeping’ gene glyceraldehyde phosphate dehydrogenase (GAPDH) was included in parallel reactions with each cDNA template to control for cDNA quality. 5 to 10% of the RT-PCR reaction products subjected to 30-40 PCR cycles were loaded onto the agarose agarose, ethidium bromide stained DNA gel pictured here. Purified DNA from the HCC library pool is included as a positive control (C) for each nucleic acid according to the invention. Two independent HCC samples (H) were included in this analysis together with one non-diseased liver sample (N) for representative nucleic acids according to the invention. M=molecular mass marker (100 bp ladder).

FIG. 4 A/B: Verification of differential gene expression by RNA blots

Independent evaluation of RNA samples from a pool of 3 non-diseased livers (L) and from 2 HCC tissues (H) verifies the increased expression of OBcl1 (SEQ ID 10) and OBcl5 (SEQ ID 11) in this image of an RNA blot autoradiogram as indicated on the figure. The results from the antisense strand probes specific for each sequence (A, top; specific signal) and the corresponding sense strand probes as negative controls (B, bottom) demonstrate the specificity of hybridization with the antisense probes.

FIG. 5: OBcl5 RNA localization in HCC vs. NNL

In situ hybridization analysis detects OBcl5 RNA in hepatocellular carcinoma (HCC) and non-neoplastic liver (NNL) samples. A radioisotope-labeled antisense probe (as) hybridises specifically with OBcl5 RNA on tissue sections and is detected by development of the section with an autoradiographic emulsion. Dark spots are developed silver grains from the emulsion indicating specific hybridization to OBcl5 RNA. The complimentary sense probe (s) cannot hybridise to OBcl5 RNA in situ despite chemical similarity to the antisense probe. The sense probe therefore serves as the negative control in panels A and C where only background signal is detected. OBcl5 RNA is marginally detected in NNL shown in panel B and clearly indicated in HCC in situ as evidenced by the large number of silver grain spots in panel D. Each panel is shown with a magnifcation of 200 times (200×).

FIG. 6: siRNA-mediated knock-down of OBcl5 RNA expression

HepG2 cells were transfected with siRNA oligonucleotides specific to the OBcl5 RNA sequence or with oligonucleotides with identical composition but scrambled sequence as a negative control (Table 10). These specific oligonucleotides interact with and destablize OBcl5 RNA thereby reducing the level of this RNA in the hepatoma cells, a process known as a knockdown of OBcl5 RNA levels. Negative control scrambled oligonucleotides are used in parallel transfections to provide control reference RNA for the subsequent experimental read-out. Q-PCR was employed to determine the levels of expression of OBcl5 RNA and retinoblastoma protein 1 (RB1) mRNA in specific oligonucleotide-transfected cells (experimental) compared with scrambled oligonucleotide-transfected (control) cells from three independent experiments (A, B and C). Y axis represents log2 per cent values of OBcl5 mRNA-remaining activity (white, left columns); whereas the RB1mRNA log2 ratio values indicate the fold increase in the level of RB1 mRNA in OBcl5 siRNA transfected versus control oligo transfected HepG2 cells (black, right columns). A decrease in OBcl5 RNA mediated by the specific siRNA oligonucleotide is evident. Elevated levels of RB1 mRNA in the experimental but not in the control cells suggests that OBcl5 expression negatively regulates the level of this tumor suppressor mRNA.

FIG. 7: DAP3 protein expression in tissues

Protein extracts are subjected to immunoblot analysis with antibodies specific for DAP3 and β-actin protein to determine the level of expression of these proteins in human tissues. Following incubation with a horse-radish peroxidase (HRP) conjugated secondary antibody and detection of the immune complexes with a chemiluminescent HRP substrate, the intensities of the bands are analyzed densitometrically and each signal is normalized to the intensity of the corresponding β-actin signal. The tissues represented in each lane are defined in Table 8, which also includes the quantitative analysis of DAP3 protein levels in these tissues. These analyses indicate that DAP3 protein, the functional product of the DAP3 mRNA specifically upregulated in HCC, is also highly overexpressed in HCC.

TABLE 8
Tissues examined in FIG. 7 and densitometric quantitation
of DAP3 protein expression levels in human tissue extracts.

| No. | Tissue | DAP-3 | β-actin | DAP-3 normalized |
|---|---|---|---|---|
| 1 | brain | 1.5 | 7.4 | 1.4 |
| 2 | cerebellum | 1.6 | 7.5 | 1.4 |
| 3 | heart | 1.2 | 0.0 | 1.2 |
| 4 | colon | 3.3 | 7.4 | 3.0 |
| 5 | lung | 0.0 | 6.7 | 0.0 |
| 6 | stomach | 4.2 | 6.2 | 4.6 |
| 7 | pancreas | 16.3 | 6.2 | 17.8 |
| 8 | kidney | 0.0 | 0.0 | 0.0 |
| 9 | prostate | 0.9 | 4.2 | 1.5 |
| 10 | uterus | 1.4 | 9.2 | 1.0 |
| 11 | HCC2 | 20.7 | 6.3 | 22.2 |
| 12 | HCC3 | 31.1 | 8.0 | 26.3 |
| 13 | HCC4 | 15.6 | 6.9 | 15.3 |
| 14 | liver | 1.9 | 3.5 | 3.7 |
| 15 | skeletal muscle | 0.1 | 0.0 | 0.1 |
| 16 | testis | 0.5 | 6.0 | 0.6 |
| 17 | spleen | 0.0 | 5.1 | 0.0 |
| 18 | mammary gland | 0.4 | 8.1 | 0.3 |

FIG. 8: Expression of HCC deregulated genes correlates with proliferation of hepatoma cells

Proliferation-dependent expression of target gene sequences according to the invention in hepatoma cells (Hep3B) following serum stimulation for 8 hours (black columns) and for 12 hours (white columns) of quiescent cells. The log2-transformed ratios of serum-stimulated vs. quiescent expression values from a cDNA microarray experiment readout is provided. The substantial increase in the level of expression of these sequences in proliferating compared to quiescent hepatoma cells suggests that these sequences are functionally significant for liver cancer cell growth.

### EXAMPLES

### Example 1

### Preparation of HCC Subtracted cDNA Libraries

RNA is isolated from three pathologist-confirmed HCC tumor samples and from three pathologist-confirmed non-diseased human liver samples using the TRIZOL reagent (Invitrogen) according to standard methods (Chomczynski & Sacchi, 1987, Anal. Biochem. 162:156-159). The tissues used for the generation of cDNA libraries is from patients that provided specific informed consent for utilization of this material for research purposes, including commercial research. mRNA is converted to double stranded cDNA with reverse transcriptase and DNA polymerase as described in the instructions provided in the “PCR select cDNA subtraction kit” from Clontech Laboratories. To enrich for cDNAs specifically increased and decreased in HCC, cDNAs expressed in common and at similar levels in the reference liver pool and in HCC are removed by subtractive suppressive hybridization (SSH) according to the instructions provided in this kit and as described by Diatchenko et al. (1996, Proc. Natl. Acad. Sci. USA 93:6025-6030). The SSH steps are performed in both directions (subtracting non-diseased liver cDNAs from HCC cDNAs and subtracting HCC cDNAs from non-diseased liver cDNAs) so the resulting cDNA molecules represent nucleic acid sequences both up- and down-regulated in HCC but do not represent those that are not differentially expressed. In addition a normalized but not subtracted HCC cDNA library is generated to better represent rare mRNA transcripts in HCC tissues. These cDNAs are separately cloned into the pCR11 vector (Invitrogen) by ligation into this plasmid followed by electrophoretic transformation into E. coli XL-1-Blue electroporation-competent cells (Stratagene). The cloning is carried out as described by the supplier of the vector and competent cells. Cloned differentially expressed cDNAs are plated onto selective (ampicillin) media to isolate individual clones. 960 clones are isolated from each SSH library and 576 clones isolated from the normalized HCC library and cultures established in 96-well microtiter plates. Together these cDNA clones provide a unique representation of mRNA expression specific for human HCC tissue.

### Example 2

### Preparation and Hybridization of HCC cDNA Microarrays

1 ml cultures of the SSH cDNA library clones described above are established and the cDNA inserts amplified by PCR with primers specific to the vector sequence flanking the cDNA inserts. The M13 forward (5′-GTAAAACGACGGCCAG-3′; SEQ ID 20) and M13 reverse primers (5′-CAGGAAACAGCTATGAC-3′; SEQ ID 21) are employed for the PCR amplification of clone inserts. Fifty microliters of the bacterial cultures are heat denatured at 95° C. for 10 minutes, debris removed by centrifugation, and 2 μl of the supernatant included in a standard PCR [1× Amplitaq PCR buffer, 2.5 mM MgCl₂, 37.5 nM each primer, 0.5 mM each of dATP, dCTP, dGTP and dTTP and 1.5 units Amplitaq DNA polymerase (Applied Biosystems)]. Reaction conditions are 95° C. for 5 minutes followed by 35 cycles of: 94° C. for 30 seconds, 60° C. for 30 seconds, 72° C. for 60 seconds; then followed by 72° C. for 7 minutes and then cooled to 4° C. Amplification of cDNA inserts is confirmed by electrophoresis of a 5% of the PCR on a 1% agarose gel comprising 0.4 μg/ml ethidium bromide and run in 1× Tris Acetate EDTA (TAE; 40 mM Tris-acetate, 1 mM EDTA, pH 7.5) buffer. Each of the SSH clone amplified insert sequences is affixed to sialinized glass microscope slides (GAPS Corning) using a GeneticMicrosystems 417 cDNA arrayer robot to generate custom HCC cDNA microarrays. The protocol for spotting the cDNA inserts to the slides is according to that published by Hedge et al. (2000, Biotechniques 29:548-560) except that PCR products are spotted directly from the PCR microtiter plates without purification or adjustment of the cDNA buffer. In addition to the SSH cDNA clone inserts, numerous control DNAs are spotted onto the microarrays as controls for hybridization reactions. Further, approximately 2000 publicly available cDNA clones corresponding to genes previously reported to be involved in cancer are purchased from the German Genome Research Center (RZPD), expanded, amplified and spotted onto these microarrays as described above. For preparation of hybridization probes, 20 micrograms of RNA from additional pathology-confirmed liver disorders and from the same quantity of pooled non-diseased liver RNA is converted to cy5-fluorescence-labeled and cy3-fluorescence-labeled cDNA, respectively (cy5-CTP and cy3-CTP, Pharmacia) using reverse transcriptase according to the standard methods (Hedge et al., 2000, Biotechniques 29: 548-560). Using this protocol, these labeled cDNAs are competitively hybridized to the HCC microarrays. Following prehybridization at 42° C. for 45 minutes in 5×SCC (0.75 M sodium citrate, 75 mM sodium citrate, pH 7.0); 0.1% SDS (sodium dodecyl sulfate) and 1% BSA (bovine serum albumin), the hybridization is carried out overnight at 42° C. in buffer comprising 50% formamide, 5×SSC, and 0.1% SDS. Hybridized slides are washed in stringent conditions (twice at 42° C. in 1×SSC, 0.1% SDS for 2 minutes each; twice at room temperature in 0.1×SSC, 0.1% SDS for 4 minutes each; and twice at room temperature in 0.05×SSC for 2 minutes each), dried and analyzed with the GeneticMicrosystems 418 cDNA microarray scanner and associated Imagene 4.1 image analysis software according to the manufacture's recommendations.

### Example 3

### Independent Verification of Differential Expression of the Nucleic Acids and Polypeptides According to the Invention

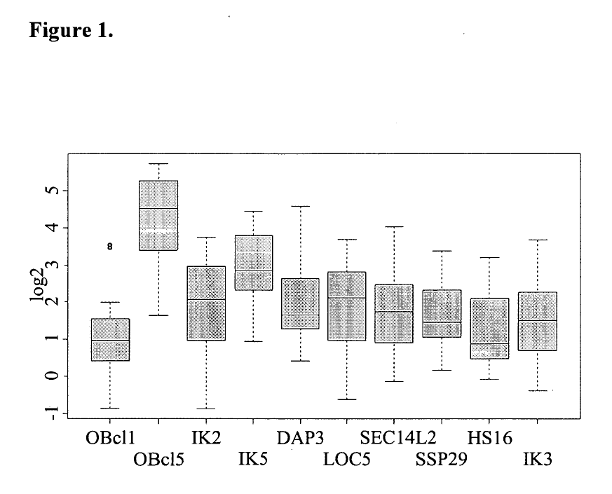

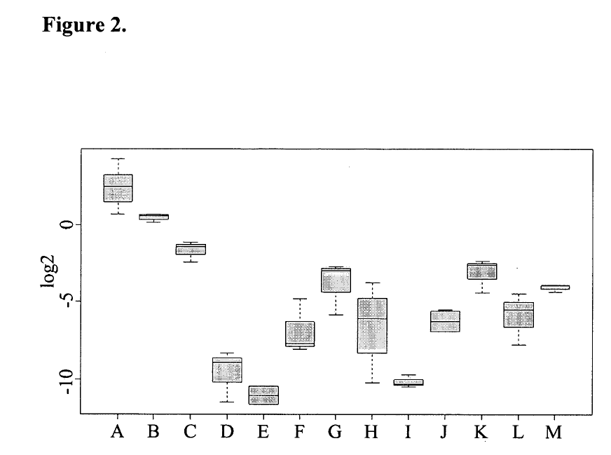

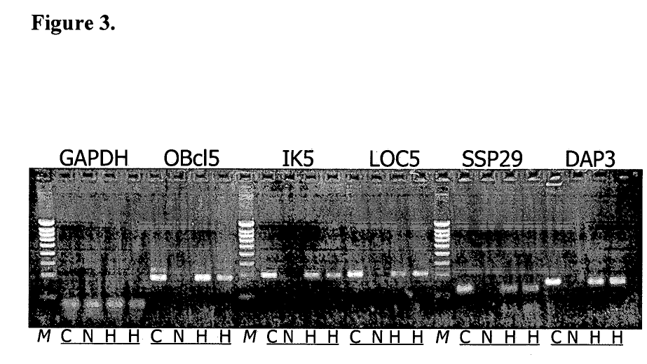

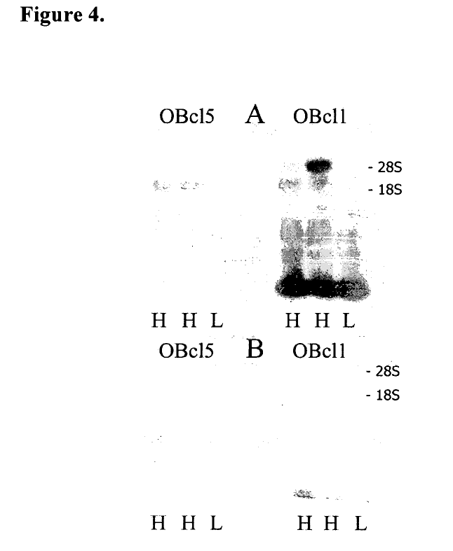

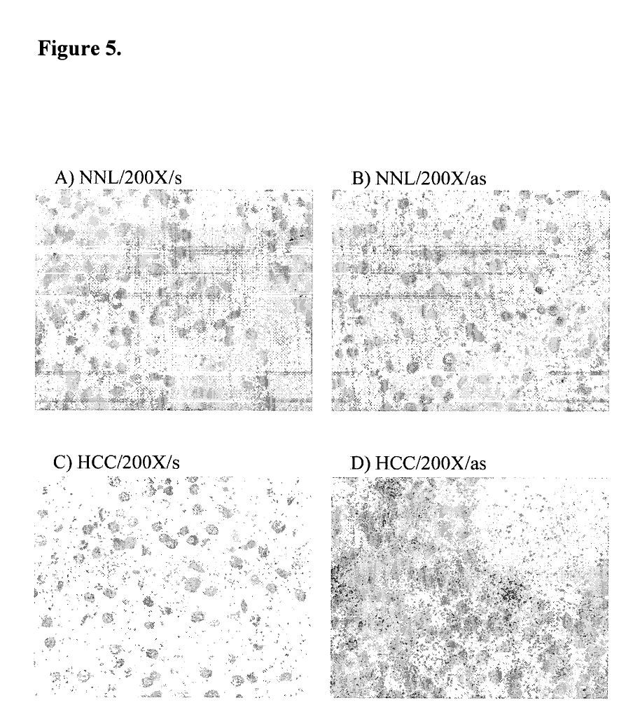

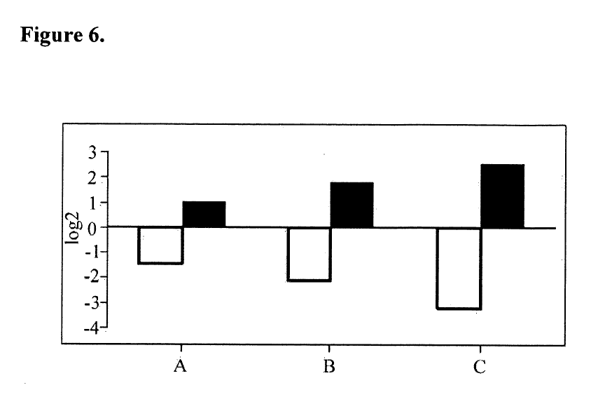

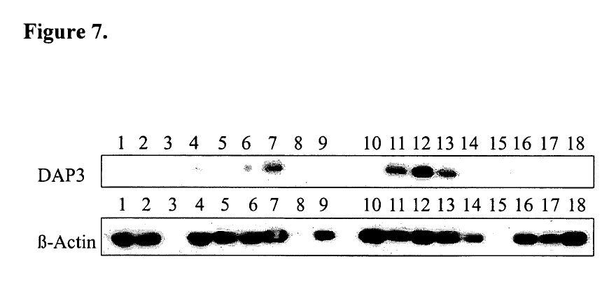

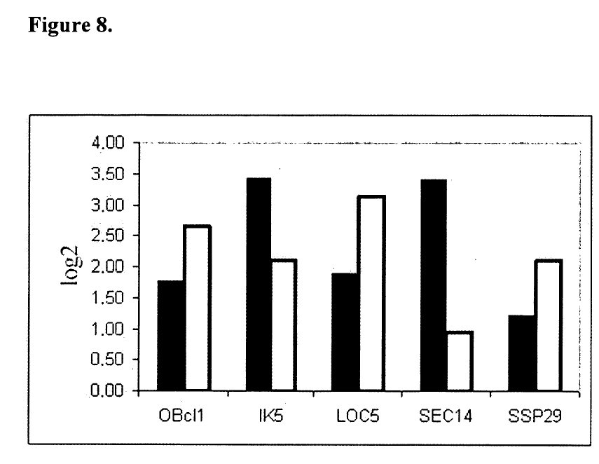

RNA is isolated from human patient samples as described in detail above. HCC samples for this analysis are not from the same patients as employed for production of the HCC SSH library or for cDNA microarray chip hybridization (see examples above, Tables 3A/3B, 4 and FIG. 1). In addition to HCC samples, RNA is prepared from independent non-diseased liver samples to assess expression of the nucleic acids according to the invention in non-diseased liver tissue. Further, RNA is prepared from additional non-diseased and cancer tissues to assess expression of the nucleic acids according to the invention in other normal human tissues and other human cancers. 1 μg of RNA is converted to single-strand cDNA with the aid of Superscript reverse transcriptase (Invitrogen) in dATP, dCTP, dGTP, and dTTP (0.4 mM each), 7.5 nM random 6-nucleotide primer (hexamers), 10 mM dithiothreitol and 1 unit RNAse inhibitor using standard procedures known in the art (Sambrook et al., Molecular Cloning, 2^{nd} ed., 1989, Cold Spring Harbor Press, NY, USA, pp. 5.52-5.55). The presence or absence of the nucleic acids according to the invention is then determined by amplification of these sequences from the cDNA with primer pairs specific to each nucleic acid according to the invention in PCR experiments. The primers used for this analysis are given in the following Table 9.

TABLE 9

RT-PCR primers with
their respective SEQ ID numbers

| | |
|---|---|
| SEQ | Primer 2 |

| | | | |
|---|---|---|---|
| Clone | ID | Primer 1 (SEQ ID) | (SEQ ID) |

| | | | |
|---|---|---|---|
| OBcl1 | 10 | 5′-CAGGTGAATTTCAAAG | 5′-GTGAGTAAATCCTCCTT |
| | | GAGGATTTACTCAC-3′ | TGAAATTCACCTG-3′ |
| | | (22) | (23) |
| OBcl5 | 11 | 5′-GCAAGCCAGGAAGAGT | 5′-TGCCAGGAAACTTCTTG |
| | | CGTCACG-3′(24) | CTTGATGC-3′(25) |
| IK2 | 12 | 5′-AGTAACCAGTTGAGAT | 5′-CAGAAGAGCAACAAGAA |
| | | GAAGCACGTC-3′(26) | TGGTATCCTGC-3′(27) |
| IK5 | 13 | 5′-AACTTGAGTTCTATTT | 5′-TTGCTTGGGTCATCTAA |
| | | ACCTTGCAC-3′(28) | AGAC-3′(29) |
| DAP3 | 14 | 5′-ACTCACGTGCAAGGAT | 5′-AGCTCTCGGACTCTCAA |
| | | GATG-3′(30) | CTG-3′(31) |
| LOC5 | 15 | 5′-CTTCTCCTATGACTGA | 5′-CAGGATGCAGAACTCAC |
| | | TCCTACTATG-3′(32) | CCTG-3′(33) |
| SEC1 | 16 | 5′-GCAGATTTCCCGTGGC | 5′-GTTGGGCAGCACCTCTG |
| 4L2 | | TCCTC-3′(34) | TCATC-3′(35) |
| SSP29 | 17 | 5′-CTGTGACATTCCGCCT | 5′-CCACGCTACTGCAAGAA |
| | | TCCTTC-3′(36) | TCTTAC-3′(37) |
| HS16 | 18 | 5′-AGAAGTTCAACCTGGA | 5′-CAAGGAAGCTAGGAATG |
| | | GAGATGG-3′(38) | ACAGGAG-3′(39) |
| IK3 | 19 | 5′-GCAAAGGCAAATTCAT | 5′-CAGATACGAACAGTGAA |
| | | GTTACTCT-3′(40) | TGGAAATACG-3′(41) |

These primers are also employable for diagnosis of disorders according to the invention, but the skilled worker may as well design other primers specific for a given nucleic acid according to the invention. The PCR included 0.5% of the cDNA, 1× Amplitaq PCR buffer, 2.5 mM MgCl₂, 37.5 nM each primer, 0.5 mM each of dATP, dCTP, dGTP and dTTP and 1.5 units Amplitaq DNA polymerase (Applied Biosystems). PCR conditions are optimized as needed for each primer pair, typically: 94° C. for 3 minutes followed by 30 cycles of: 94° C. for 15 seconds, 60° C. for 30 seconds, 72° C. for 60 seconds, then cooled to 4° C. Amplification of cDNA inserts is confirmed by electrophoresis of a 5-10% of the PCR on a 1% agarose gel comprising 0.5 μg/ml ethidium bromide and run in 1× Tris Acetate EDTA (TAE) buffer. Standard controls for RT-PCR including RNAse treatment of samples prior to cDNA synthesis and omission of reverse transcriptase routinely demonstrated the specificity of these reactions. Reactions are scored for expression (+) or absence of expression (−) based upon whether a discrete band of the correct molecular size is observed in the gel. Very faint or ambiguous bands under these conditions are scored with (+/−). A summary of these verification studies in HCC and non-diseased liver is given in Table 6. Data representative of these analyses in independent HCC and non-diseased liver samples is provided in FIG. 3.

Quantitative RT-PCR (Q-PCR) also verifies the over expression of sequences according to the invention in liver cancer and other liver disorder relative to non-diseased liver. For these studies the TaqMan hydrolysis primer strategy and the SYBR Green intercalating dye strategies were employed as described in detail in Example 5 and illustrated in FIGS. 2 and 6.

An additional independent validation of differential expression of the nucleic acids according to the invention is illustrated in FIG. 4. In this case, 15 μg of RNA from two HCC samples and from non-diseased liver is subjected to denaturing electrophoretic separation on a 1% agarose gel comprising 2.2 M formaldehyde and 1× MOPS buffer (10 mM 4-morpholinepropanesulfonic acid, 1 mM EDTA, 5 mM sodium acetate, pH 7.0) run in 1× MOPS buffer. The size-fractionated denatured RNA is transferred to nylon membrane (GeneScreen, New England Nuclear) with the RNA (northern) blot technique and cross linked to the membrane with UV light, all according to procedures well known to the skilled artisan (Sambrook et al., Molecular Cloning, 2^{nd} ed., 1989, Cold Spring Harbor Press, NY, USA, pp 7.39-7.52). cDNA clone inserts of from the SSH clones for OBcl1 and OBcl5 (SEQ ID Nos. 10 and 11) are isolated by PCR amplification as described in the previous example. Single stranded radiolabeled RNA probes corresponding to these sequences are synthesized from this template using SP6 and T7 RNA polymerase in the presence of α-³²P-UTP in 1× labeling buffer: 0.5 mM ATP, CTP, GTP, 10 mM dithiotreitol, and 20 units of appropriate RNA polymerase at 37° C. for 35 minutes. The resulting antisense probe is complementary to the corresponding mRNA sequence and thus expected to hybridize specifically to the mRNA sequence on the northern blot. Conversely, the sense probe sequence matches that of the mRNA and thereby not to hybridize to the mRNA. Identical northern blots are prehybridized in 15 ml of 250 mM monobasic sodium phosphate, 250 mM dibasic sodium phosphate, 7% SDS, 1 mM EDTA and 1% BSA for at least 30 minutes at 68° C. For hybridization the prehybridization buffer is removed and replaced with 10 ml of the same buffer including the sense and each antisense RNA probes described above at 68° C. overnight. The RNA blots are washed under stringent conditions (2×SSC, 0.1% SDS twice at room temperature for 15 minutes each; 1×SSC, 0.1% SDS twice at 68° C. for 10 minutes each), dried and exposed to x-ray film to produce an autoradiograph. As seen in FIG. 4, each antisense probe specifically hybridizes to discrete HCC RNA but only weakly or not at all to non-diseased liver RNA. The specificity of these results is demonstrated by the absence of specific signal from the corresponding sense probe for both OBcl1 and OBcl5. In addition, RNAs of different molecular weights are apparent with the OBcl1 antisense probe. This result most likely represents discrete mRNA species, perhaps produced by alternative splicing. These species are expected based upon the finding that several different sized cDNA clones corresponding to this sequence are reported in the GenBank sequence database.

Furthermore, in situ hybridization reveals strong OBcl5 RNA expression in HCCs when compared to NNL tissue samples. According to the protocol of Fickert et al. (Am J Pathol. 2002 February; 160 (2): 491-9.), S³⁵-labeled probes are synthetized as described above for RNA blot. The templates for in vitro transcription are amplified from a plasmid comprising OBcl5 3′ cDNA. Primers employed to generate the in vitro transcription templates (MWG Biotech, Munich, Germany) are OBcl5-p6 forward primer (5′-aatctgcaagccaggaagagt-3′, SEQ ID 48) and M13 for (5′-gtaaaacgacggccag-3′, SEQ ID 20) for the T7 antisense probe spanning 365 bases of OBcl5 RNA including both exons (SEQ ID 11 from nucleotide 95 to 484); and M13rev (5′-caggaaacagctatgac-3′, SEQ ID 21) and OBcl5-p7 reverse primer (5′-tctagtttcagttttgatgatattttg-3′, SEQ ID 49) for the SP6 sense probe spanning 436 bases of OBcl5 sequence including both exons (SEQ ID No 11 from nucleotide 436 to 1). To amplify these templates PCRs include 10 pM forward primer, 10 pM reverse primer, 1 pM dNTP's (Invitrogen), PCR buffer II, 5 mM MgCl₂ (Applied Biosystems, Foster City, Calif.), 217 ng of template plasmid DNA, 2.5 U AmpliTaq polymerase (Applied Biosystems, Foster City, Calif.) are performed with Applied Biosystems Gene Amp PCR System 270, usually: 94° C. for 3 minutes, 94° C. for 30 seconds, 50° C. for 30 seconds, 72° C. for 50 seconds, the last 3 steps are repeated 25 times followed by 72° C. for 3 minutes, then added for the final extension. The amount of DNA in PCR products is determined by spectrometry with a Smart Spec 3000 (BIO-RAD, Hercules, Calif.). In vitro transcription assay is carried out at 37° C. for 2 hours, using 200 ng of each template in transcription buffer (Boehringer Mannheim, Germany), 100 mM dithiothreitol (DTT), 1 mM each of rNTP, RNAse Inhibitor (Eppendorf, Hamburg, Germany), α-S³⁵—UTP (Amersham Bioscience), RNA-polymerase SP6/T7 (Boehringer Mannheim). After removal of unincorporated nucleotides (Rnase-free MicroSpin S-200 HR column Amersham Bioscience, Buckinghamshire, UK), template DNA is digested with 2 units of RNase-free DNase for 10 minutes at 37° C. To obtain an average probe size of 150 bp hydrolysis is performed at 60° C. for 42 minutes, using hydrolysis buffer (400 mM NaHCO₃, 600 mM Na₂CO₃, 100 m M DTT) and neutralized in 0.1M sodium acetate, 10 mM DTT and 1% glacial acetic acid. The transcript probes are precipitated with LiCl/isopropanol and resuspended in 50% formamide comprising 25 mM DTT. Paraffin-embedded histologicaly-verified samples of HCC's and non-neoplastic normal liver sections are cut at 2.5 micrometers a Microm HM 355S microtome (Microm, Walldorf, Germany) and mounted 2 sections per slide onto Superfrost slides (Menzel-Gläser, Braunschweig, Germany). All sections are dried overnight Dry sections are heated to 60° C. for 1 hour and deparaffinized in Xylene for 30 minutes. Rehydration is performed in graded ethanols of 100%, 90%, 70% and 50% followed by washing of 4 times for 3 minutes in Tris-buffered saline (TBS buffer) and sections are then fixed in phosphate-buffered saline (PBS buffer) comprising 4% paraformaldehyde. After several PBS washes, sections are denatured in 0.2M HCl for 10 minutes and washed again 4 times for 3 minutes each in TBS. The protein digest is performed in 20 μg/ml of RNase-free proteinase K (F. Hoffman La Roche Ltd. Basel, Switzerland) in TBS comprising 2 mM of CaCl₂ at 37° C. for 20 minutes. The reaction is stopped by incubation of slides for 5 minutes in TBS at 4° C. Subsequently sections are washed again 3 times for 4 minutes in TBS buffer at room temperature and incubate in 0.1M Tris buffer pH 8 comprising acetic anhydride for 10 min. The sections are dehydrated in graded ethanols of 50%, 70%, 90%, 100% and finally in chloroform, and left to air-dry for 2 hours. For hybridisation a labelled probe (1×10⁶ cpm per section; probe radioactivity determined with LKB Wallac, 1211 RACKBETA Liquid Scintillation Counter is diluted in 50 μl/section hybridization buffer comprising 12.5 mM phosphate buffer pH 6.8, 12.5 mM Tris, 0.4M NaCl, 3 mM EDTA, 1.25× Denhardts solution, 50% formamide, 12.5% dextran sulphate, 0.1M DTT, 100 nM S-rATP (Boehringer Mannheim), 60 ng of yeast tRNA, and 20 ng of poly(A) (Boehringer Mannheim). Sections are hybridized overnight at 52° C. in a humid chamber comprising 2× standard saline citrate (SSC) pH 7, and 50% formamide. Next, sections are washed with formamide buffer (10 mM phosphate buffer pH 6.8, 10 mM Tris-HCl pH 7.7, 0.3 M NaCl, 5 mM EDTA, 0.1× Denhardts solution, 0.07% β-mercaptoethanol, and 50% formamide) twice, for 1 hour and 2 hours, respectively. Thereafter sections are washed twice for 15 minutes in 10 mM Tris-HCl pH 7.4, 0.5M NaCl, 2.5 mM EDTA and 0.07% β-mercaptoethanol. RNase treatment is carried out in the same buffer comprising 20 μg/ml RNase A (Boehringer Mannheim) at 38° C. for 30 minutes followed by further washing with formamide washing buffer at 37° C. overnight. The sections are subsequently washed in 2×SSC and 0.07 β-mercaptoethanol for 30 minutes at 45° C., followed by another 30 minutes at 45° C. in 0.1×SSC and 0.07 β-mercaptoethanol. Thereafter sections are dehydrated in graded ethanols of 50%, 70%, 90%, 100% and air-dried. Finally, the slides are coated in Ilford K2 photo emulsion (Ilford Ltd. Mobberly, Cheshire, UK). After 10, 14 and 17 days of exposure, development is carried out using Kodak Dl 9 developer (Eastman Kodak, Rochester N.Y.). The sections are counterstained with hematoxilin and mounted in aqeous mounting media (Aquatex-EM Science, Gibbstown, N.J.). Dark spots are developed, silver grains from the emulsion indicating specific hybridization to OBcl5 RNA (FIG. 5). The complimentary sense probe(s) cannot hybridize to OBcl5 RNA in situ despite chemical similarity to the antisense probe. The sense probe therefore serves as the negative control (FIG. 5, in panels A and C) where only background signal is detected. OBcl5 RNA is marginally detected in NNL (shown in FIG. 5, in panel B) and clearly indicated in HCC in situ as evidenced by the large number of silver grain spots (FIG. 5, in panel D.

Furthermore, the protein expression analyses indicate that for example DAP3 protein, the functional product of the DAP3 mRNA specifically upregulated in HCC, is also highly overexpressed in HCC. To detect DAP3 protein expression in various tissues standard western blot analysis is performed using protein extracts derived from frozen tissues (stored in liquid nitrogen), see FIG. 7. The 50 μm sections are obtained (HCC, normal liver and various organ samples) using a refrigerated microtome (cyrocut, Leica CM3050), wherein the identity and homogeneity of the tissues under scrutiny is verified by H&E-staining of sections taken before, in between and after each cutting process. Tissues sections are resuspended in ice-cold RIPA-buffer (50 mM Tris-HCl pH 7.4, 250 mM NaCl, 0.1% SDS, 1% deoxycholate, 1% NP-40) supplemented with 2 μg/ml leupeptin, 2 μg/ml pepstatin, 2 μg/ml aprotinin, 1 mM phenylmethylsulfonylfluoride (PMSF), and 2 mM dithiothreitol followed by homogenization through sonication (2 bursts of 5 seconds) on ice. After incubation for 20 minutes on ice, the lysates are cleared by two centrifugational steps in a microcentrifuge at 13 000 rpm for 15 minutes at 4° C. and the supernatants are collected. Protein concentrations are determined by the Bradford assay (Biorad) using bovine serum albumin as a standard. Equal amounts of protein (typically 10-30 μg) are separated on a 12% SDS-PAGE gel and transferred electrophoretically to a polyvinylidene diflouride (PVDF) membrane (Hybond-P, Amersham) through Semidry-blotting (TE 70, Amersham). The membrane is blocked for 1 hour at room temperature in blocking solution [5% milk in TBS-T (25 mM Tris-HCl pH 7.4, 137 mM NaCl, 3 mM KCl, comprising 0.1% Tween-20)] and incubated with the primary antibody solution (prepared in TBS-T/1% milk) at 4° C. overnight with agitation. Antibodies specific for the following antigens are used: DAP3 (1:1000; BD Transduction Laboratories), and β-actin (1:5000, Sigma). After removal of the primary antibody solution and several washes in TBS-T, the membrane is incubated with a HRP (horse-radish peroxidase)-conjugated secondary antibody (rabbit anti-mouse, 1:1000; Dako) for one hour at room temperature. Following several washes in TBS-T, detection is performed through chemiluminiscence (ECL, Amersham) and exposing to x-ray film (FIG. 7). The intensities of the bands are analysed densitometrically using ChemiImager 5500 software (Alpha Innotech) and each signal is normalised to the intensity of the corresponding β-actin signal (Table 8).

These data provide independent verification of deregulated expression of the nucleic acids and polypeptides according to the invention in HCC. Expression of the nucleic acids and polypeptides according to the invention is either absent or observed only at very low levels in non-diseased liver, thereby validating the differential expression of these nucleic acids identified by hybridization to the cDNA microarray. The results provide surprising evidence that the nucleic acids and polypeptides according to the invention can be used to diagnose, prevent and/or treat disorders according to the invention.

### Example 4

### Sequences According to the Invention are Increased in Proliferating Liver Cancer (Hepatoma) Cell Lines

Human hepatoma cell lines (HepG2, Hep3B) are cultured in DMEM supplemented with 10% fetal bovine serum (FBS) in a humidified incubator with 5% CO₂ at 37° C. The cells are split to about 20% confluency and subsequently rendered quiescent by culturing in the absence of serum for 3 days. After the starvation period, the cells are stimulated to proliferate by the addition of 10% FBS to the media. Samples are taken before and following the induction of cell growth (0, 8 and 12 hours) for the preparation of RNA and for determination of the position of the cells in the cell cycle by FACS (fluorescence activated cell sorting) analysis. Accordingly, to determine the cell cycle distribution by propidium iodide (PI) staining, the cells are harvested by trypsinization, washed twice with phosphate buffered saline (PBS) and finally resuspended in 500 μl PBS. Subsequently, 5 ml prechilled methanol is added. After 10 minutes incubation at −20° C. the cell suspension is directly used for FACS analysis following 3 times washing in PBS, resuspended in 5001 μl propidium iodide (PI) staining buffer (DNA-Prep Stain, Part No. 6604452; Beckman Coulter) and incubated for 15 minutes at 37° C. Finally, 70 μl of 1M NaCl is added and the samples are kept on ice protected from light prior to analysis on an EPICS XL-MCL flow cytometer (Beckman Coulter). Cells prepared from an asynchronous cell population are used as reference.

The isolated RNA is used to monitor the expression of genes in quiescent vs. proliferating hepatoma cells by cDNA microarray analysis. Following labeling with fluorescent dyes as described in example 2, the RNAs are hybridized on a specifically developed HCC-specific cDNA microarray chip that also contained control genes which are known to be expressed in a cell cycle dependent manner. Finally, the data are analysed using ImaGene 4.1 and GeneSight software packages. The signals obtained for 0 hours samples isolated before the addition of serum are used as reference. The log2-transformed ratios of serum-stimulated vs. quiescent expression values from the cDNA experiment readout is provided in FIG. 8.

These data indicate that the sequences according to the invention are correlated with human liver tumor cell proliferation. Compared to the state of the art, these nucleic acids and polypeptides therefore surprisingly allow improved, more sensitive, earlier, faster, and/or non-invasive diagnosis of the liver disorders and/or epithelial cancers.

### Example 5

### Functionally Significant Role for Elevated Expression of Sequences According to the Invention in Liver Disorders, Especially Liver Cancer

Detailed sequence analyses revealed sequence similarities between OBcl5 RNA and eukaryotic non-coding RNAs. In addition, multiple attempts with diverse methodologies to detect a protein product from this RNA have not revealed such a product. Therefore, this RNA may be not translated into a polypeptide but may itself have functional (e.g., regulatory) properties. Using a protocol according to TransMessenger Transfection Reagent Handbook (Qiagen, 10/2002), reduction of the level of OBcl5 RNA in proliferating human hepatoma cells with small interfering RNA (siRNA) oligonucleotides (siRNA mediated knock-down of OBcl5 RNA) is performed. Double stranded small interfering RNA (siRNA) oligonucleotide probes (Table 10) are designed for in situ depletion of RNA levels corresponding to OBcl5 (SEQ ID 11) and provided by Qiagen.

TABLE 10

Double stranded small interfering
RNA (siRNA) oligonucleotide probes

| | | |
|---|---|---|
| Name | Sequence | SEQ ID* |

| | | |
|---|---|---|
| OBc15 | 5′ r(UCUGCAAGCCAGGAAGAGU)d(TT) 3′ | 50 |
| siRNA fw | | |
| OBc15 | 5′ r(ACUCUUCCUGGCUUGCAGA)d(TT) 3′ | 51 |
| siRNA rev | | |
| OBc15 | 5′ r(CCUCCAGAACUGUGAUCCA)d(TT) 3′ | 52 |
| siRNA fw1 | | |
| OBc15 | 5′ r(UGGAUCACAGUUCUGGAGG)d(TT) 3′ | 53 |
| siRNA rev1 | | |
| DAP3 | 5′ r(CUACAAAUGAGCGCUUCCU)d(TT) 3′ | 54 |
| siRNA fw | | |
| DAP3 | 5′ r(AGGAAGCGCUCAUUUGUAG)d(TT) 3′ | 55 |
| siRNA rev | | |

[*sequences listed with SEQ ID numbers in accompanying information for these siRNA ribo-oligonucleotides do not include the two 3 ′deoxyribonucleotide (dTT) “tail” at the end of each sequence as it is not possible to designate a ribonucleotide/deoxyribonucleotide chimeric molecule in these listings].

HepG2 cells (with density of 3×10⁴ cells per well) are seeded and incubated for 24 hours at 37° C. and transfected with 1.5 μl of Oligofectamine Reagent (Invitrogen) and 2.5 μl of a 20 μM double stranded siRNA oligonucleotide stock solution according to the manufacturers instruction (Invitrogen protocol). After 24 hours incubation total RNA is isolated and reverse transcribed to cDNA as described in Example 2. The PCR product is monitored accordingly by incorporation of fluorescently labeled primers or various fluorescence-based indicators of including the Taqman probe hydrolysis systems and fluorescent double-stranded DNA intercalating molecules such as SYBR green. Experiments are performed according to the manufacturers instructions (GeneAmp® 5700 Sequence detection System, User Manual; PE Biosystems). Accordingly, real-time quantitative RT-PCR analyses based on TaqMan methodology are performed using the 5700 Sequence Detection System (Applera) as follows: 500 ng of total RNA is reverse transcribed as described in Example 3 and a 1:4 dilution of this cDNA template used for Q-PCR (corresponding to 6.25 ng RNA), including 5-8 pmol/μl of each primer in 30 μl of final volume. Temperatures for Q-PCR are used according to the manufacturer's instructions using 40 cycles. Triplicate reactions are performed.

Real-time Q-PCR analyses based on SYBR-Green methodology are performed using the 7000 Sequence Detection System (Applera). The PCR is performed with the SYBR-Green Universal PCR Master Mix (Applera) using cDNA corresponding to 6.25 ng RNA as above, and empirically determined amounts of each primer (RB and β-actin, 10 pmol of each primer in the reaction samples) in a 30 μl final volume according to the manufacturers instruction. Temperatures for SYBR-RT-PCR are used according to the instruction manual. These reactions are also cycled 40 times and triplicate reactions are performed. The percentage of knockdown of the target RNA levels (in this case OBcl5 RNA) is determined by Q-PCR using parallel Q-PCR determination of GAPDH or β-actin mRNA levels as a reference in either TaqMan-based (GAPDH primers used=GAPDH-p1, SEQ ID 56; GAPDHP2, SEQ ID 57; GAPDH-p3, SEQ ID 58) (β-Actin primers used=bActin-p1, SEQ ID 59; bActin-p2, SEQ ID 60; bActin-p3, SEQ ID 61) or SYBR Green analyses (β-Actin primers used as reference for SYBR green analyses=bActin-p4, SEQ ID 62; bActin-p5, SEQ ID 63) as described previously. Changes in RNA levels are determined according to the methods described by Pfaffl (Nucleic Acids Research (2001) May 1, 29(9):e45).

In such an experiment in which the level of OBcl5 RNA is knocked down in heptoma cells, it is determined that the level of mRNA encoding the tumor suppressor gene retinoblastoma protein 1 (RB1) is up-regulated several fold upon decreasing the level of OBcl5 RNA, in a dose-dependent fashion (FIG. 6) (RB1 Q-PCR primers used ═RB1-p1, SEQ ID 64; RB1-p2, SEQ ID 65). The clear conclusion is that elevated expression of OBcl5 RNA in HCC may provide a negative regulation of the RB1 and therefore facilitate tumor cell growth. Thus, reduction of the level of OBcl5 RNA (knock-down) in proliferating human hepatoma cells with siRNA oligonucleotides supports a functionally significant role for elevated expression of OBcl5 RNA in liver disorders, especially liver cancer.

A further such experiment in which siRNA oligonucleotides were designed to knock-down DAP3 mRNA (SEQ ID 14) in hepatoma cells provided surprising morphological effects (oligo sequences used for DAP3 siRNA knockdown studies provided in Table 10). In the DAP3 siRNA oligo treated cells but not in cells treated identically except that other siRNA oligos were employed (such as a scrambled sequence siRNA oligo control), a substantial change in cellular morphology was observed that included enlargement of the cell volume. These treated cells remained adherant to the culture substrate but it was further observed that RNA and protein could not be extracted from such treated cells using the standard methods described in these examples. The lack of such effects with similar siRNA oligo treatments in parallel in identically treated hepatoma cells argues that these observations are specific to knockdown of DAP3 mRNA levels in the hepatoma cells. Over expression of DAP3 mRNA therefore may be critical for liver cancer cell viability. These observations further support a functionally significant role for DAP3 in liver tumor cells.

These results provides further surprising evidence that the nucleic acids and/or polypeptides according to the invention can be used to diagnose, prevent and/or treat disorders according to the invention.

### Example 6

### A Method of Diagnosing Using HCC Specific Probes

A diagnostic method for disorders according to the invention preferably based on the polymerase chain reaction (PCR) can be established. A standard PCR detection of nucleic acid sequences of the invention can be sufficient to identify, for example, circulating HCC tumor cells in the blood stream of the patient. Detection of expression of nucleic acid sequences of the invention in tumor biopsy material however, such as from a fine needle biopsy, would also be a preferred indication for this diagnostic procedure. Nucleic acid sequences of the invention, OBcl5 (SEQ ID 11) for example, are not detected in most non-diseased tissues and relatively specifically expressed in e.g. HCC. Elevated expression of this nucleic acid in cirrhosis and HCC is also demonstrated indicating the potential discriminatory power of such an approach for differential diagnosis of liver diseases (FIGS. 1, 2, 5 and Tables 5A/B).

The PCR diagnostic would preferably require approximately 1 pg, preferably at least 100 ng, more preferably at least 1 μg of RNA isolated from patient material. In the preferred utilization the RNA would be isolated according to standard procedures from e.g. the white blood cell fraction preferably from circulating blood obtained by the minimally invasive venupuncture procedure. In this preferred case, the procedure would detect the presence of HCC tumor cells in the blood circulatory system. RNA could similarly be isolated from liver biopsy material.

For specific detection of OBcl5, for example, the PCR diagnostic would include several primers specific for OBcl5 nucleic acid sequence, including a specific antisense primer (Primer OBcl5-p1; 5′-GCCACAGGTTGAACACTTAATTTG-3′; SEQ ID 42; from nucleotide 350-327 on SEQ ID 11) for cDNA synthesis from the RNA generated from the patient sample. Similarly specific PCR primers such as for example OBcl5-p2 (5′-AGGAAGAGTCGTCACGAGAACC-3′; SEQ ID 43; from nucleotide 107-128 on SEQ ID 11) and OBcl5-p3 (5′-ATAATGCTGTGCTTAGTTTATTGCC-3′; SEQ ID 44; from nucleotide 313-289 on SEQ ID 11). Sensitivity, specificity and quality control may be improved by the provision of an additional primer set (for example: OBcl5-p4; 5′-GATCGTGGACATTTCAACCTC-3′; SEQ ID 45; from nucleotide 147-167 on SEQ ID 11 and OBcl5-p5; 5′-TCTTGCTTGATGCTTTGGTC-3′; SEQ ID 46; from nucleotide 280-261 on SEQ ID 11) that are specific for the OBcl5 nucleic acid insert and internal (nested) to primers OBcl5-p2 and -p3. Quantitative assessment of OBcl5 mRNA levels may also be achieved in such detection strategies as illustrated in FIG. 2 using TaqMan Q-PCR with, for example:
OBcl5-8, SEQ ID 66 (5′-ATCTGCAAGCCAGGAAGAGTC-3′); OBcl5-p9, SEQ ID 67 (5′-CTTGCTTGATGCTTTGGTCTGT-3′); and OBcl5-p10, SEQ ID 68, (5′-CCAGACCATGCAGGAACTCTGATCGTGGAC-3′).

cDNA may be prepared from the patient RNA sample following digestion of the RNA with RNAse-free DNAse-1 (Roche) to eliminate potential contamination by genomic DNA. This contamination possibility is further controlled by including primers for PCR amplification from sequences of different exons of the OBcl5 gene such that PCR products resulting from a genomic DNA template (and thereby not reflective of expression of the mRNA corresponding to OBcl5) would be larger than the RNA specific PCR products. cDNA synthesis can e.g. be primed by the OBcl5 specific OBcl5-p1 (at about 1 μM) with the aid of reverse transcriptase [such as Maloney murine leukemia virus reverse transcriptase (Roche) at about 2 unit/reaction] in an appropriate buffer such as 50 mM Tris-HCl, 6 mM MgCl2, 40 mM KCl, and 10 mM dithiotreitol, pH 8.5. Also required in the cDNA synthesis reaction is dATP, dCTP, dGTP and dTTP, each at about 1 mM, RNAse inhibitor, such as placental RNAse inhibitor (Roche) at about 1-10 units/reaction. cDNA synthesis would be preferably carried out at 42° C. for 30 to 60 minutes followed by heating at 95° C. for 10 minutes to denature the RNA template. The resulting cDNA can be employed as the template for a PCR to detect OBcl5 in the blood (or liver biopsy sample). The additional reagents required for PCR detection of OBcl5 would preferably also be provided including: 10× Taq DNA polymerase buffer (500 mM Tris-Cl pH 8.3, 25 mM MgCl₂, 0.1% Triton X-100); a mixture of dATP, dCTP, dGTP and dTTP for a final concentration of 0.2 mM each; Taq DNA polymerase (2.5 U/reaction), and OBcl5 specific primers such as OBcl5-p2, OBcl5-p3, OBcl5-p4, and OBcl5-p5 (0.1-1 μM final concentration). A positive control for PCR amplification such DNA from a plasmid clone with the OBcl5 sequence insert would preferably also be included (1-10 ng/reaction). The PCR can e.g. be carried out over 22-40 cycles of 95° C. for 30 seconds, 60° C. for 30 seconds, 72° C. for 60 seconds. As indicated above, preferred additional sensitivity and specificity may be achieved in this diagnostic procedure by utilization of the additional OBcl5 primer set located within the sequence amplified with the original PCR primer set. In this case a subsequent PCR under conditions similar to those utilized in the first PCR reaction except that preferably primers OBcl5-p4 and OBcl5-p5 would be employed to amplify the nested sequence in a reaction that included 1-10 μl of the first PCR as the template DNA. Alternatively, the reaction may preferably be carried with the first primer set (OBcl5-p2 and OBcl5-p3) for 10-15 cycles after which and 1-10 μl of this reaction then included as template in a new PCR reaction with primers OBcl5-p4 and OBcl5-p5 (and including all the necessary PCR components). Detection of OBcl5 specific PCR product(s) should preferably utilize agarose gel electrophoresis as is known in the art and described in previous examples. Included in the diagnostic should preferably be a comparable fluid or tissue extract as a control for such PCR-based diagnostic test. This may include serum or plasma from non-diseased individuals and/or serum, plasma or tissue extracts from an appropriate animal model. If the PCR-determined expression of the nucleic acid according to the invention such as the product of the reaction with primers OBcl5-p4 and OBcl5-p5 is upregulated in the sample isolated from the patient relative to the control and if in particular the upregulated expression essentially matches the disorder specific (mean) expression ratios such as those illustrated in FIG. 1 then such matching is indicative of the patient suffering from the disorder.

Variations on this approach can also be appreciated. The cDNA synthesis and PCR amplifications can be carried our sequentially or simultaneously in a single reaction vessel utilizing heat stabile DNA polymerases with reverse transcriptase activities, such as provided by the Titan one-tube or Carboxydothermus DNA polymerase one-set RT-PCR systems from Roche. Alternatively the PCR product can be monitored by incorporation of fluorescently labeled primers or various fluorescence-based indicators of PCR product including the Taqman probe hydrolysis systems, as described above and with fluorescent double-stranded DNA intercalating molecules such as SYBR green. The fluorescent-based approaches provide advantage as the accumulation of PCR product can be continuously monitored to achieve sensitive quantitative assessment of expression of the nucleic acid according to the invention. This should be particularly advantageous for nucleic acids increased in blood or tissues of disorders according to the invention but also present at lower levels in non-diseased patients and tissues such that quantitative information about the level of expression of the nucleic acid is acquired. Further, as with this example, accurate quantitation of nucleic acid expression levels contributes to differential diagnosis, between cirrhosis and HCC for example. Comparison of this data with supplied standards indicative of disease and absense of disease provides an important advantage for such a diagnostic procedure.

Additional variations on this diagnostic strategy include simultaneous detection of multiple nucleic acids according to the invention and/or of nucleic acids according to the invention together with other nucleic acids implicated in the disorder. Further hybridization-based diagnostic detection of nucleic acids according to the invention is also envisioned. In this case mRNA detection preferably utilizing RNA blot, RNAse protection or in situ hybridization on patient cells or tissue biopsy samples is also effective.

By similar methods and variants thereof the nucleic acids according to the invention and/or of nucleic acids according to the invention together with other nucleic acids can be utilized for diagnosis of the disorders according to the invention.

### Example 7

### A Method of Diagnosing Via Antibody Detection of Polypeptides According to the Invention

A preferred diagnostic method for disorders according to the invention is based on antibodies directed against a polypeptide according to the invention. For example, a diagnostic procedure may preferably employ serum detection of specific upregulated gene proteins via enzyme-linked immunosorbent assay (ELISA) assay. In a simple form the diagnostic assay preferably includes a microtiter plate or strip of microtiter wells, e.g., thoroughly coated with an isolated and purified antibody specific to a polypeptide according to the invention such as OBcl5.pr (SEQ ID 2) or DAP3 (SEQ ID 5). The antibody may for example be an affinity purified polyclonal antibody, such as is commonly raised in rabbits, for example, or a purified monoclonal antibody such as is commonly produced in mice according to procedures well established in the art (Cooper, H. M. & Paterson, Y., (2000), In Current Protocols in Molecular Biology (Ansubel, F. A. et al., eds.) pp. 11.12.1-11.12.9, Greene Publ. & Wiley Intersci., NY); (Fuller S. A. et al., (1992), In Current Protocols in Molecular Biology (Ansubel, F. A. et al., eds.) pp. 11.4.1-11.9.3, Greene Publ. & Wiley Intersci., NY). Preferably, the antibody may a recombinant antibody obtained from phage display library panning and purification as has been described by Knappik et al. (2000, J. Molec. Biol. 296:57-86) or by Chadd and Chamow (2001 Curr. Opin. Biotechnol. 12:188-94) or a fragment thereof. The antibody coating is preferably achieved by dilution of the anti-OBcl5.pr antibody or anti-DAP3.pr antibody to 1-100 μg/ml in a standard coating solution such as phosphate buffered saline (PBS). The antibody is preferably bound to the absorptive surface of the microtiter well (such as a Nunc Maxisorp immunoplate) for 60 minutes at 37° C., or overnight at room temperature or 4° C. Prior to binding sample to the coated wells, the wells are preferably thoroughly blocked from non-specific binding by incubation for 15-60 minutes at room temperature in a concentrated protein solution such as 5% bovine serum albumin in phosphate buffered saline or 5% non-fat dry milk powder resuspended in the same buffer. Preferably, the patient sample material is then applied to the microtiter wells, diluted into the blocking solution to increase specificity of detection. The sample may be for example plasma or serum or protein extract from tissue biopsy or surgical resection prepared according to methods well known in the art (Smith, J. A. (2001) In, Current Protocols in Molecular Biology, Ausubel, F. A. et al., eds) pp. 10.0.1-10.0.23, Greene Publ. & Wiley Intersci., NY). In particular, the patient sample is brought into contact with the antibody-coated well for 30-120 minutes (or longer) at room temperature or at 4° C. Nonspecifically interacting proteins are preferably removed by extensive washing with a standard wash buffer such as 0.1 M Tris-buffered saline with 0.02-0.1% Tween 20, for example. Washes are preferably carried out for 3-10 minutes and repeated 3-5 times. Detection of DAP3.pr polypeptide in the patient sample is for example achieved by subsequent binding reaction with a second, independent anti-DAP3.pr antibody, generated as described above, recognizing a distinct epitope on the DAP3.pr polypeptide in the standard two-site ‘sandwich’ type ELISA. Binding of the second anti-OBcl5.pr antibody or DAP3.pr antibody is for example achieved by incubating the wells in the antibody (at a concentration of 1-100 μg/ml in blocking solution, for example) at room temperature for 30-60 minutes followed by extensive washing as in the previous step. The second antibody may preferably be directly coupled to an enzyme capable of producing a colorigenic or fluorgenic reaction product in the presence of an appropriate substrate, such as alkaline phosphatase. Alternatively, for example an anti-species and anti-isotype specific third antibody, so coupled to an enzyme, is employed to generate a reaction product that preferably can be detected in a standard spectrophotometric plate reader instrument. For the reaction product development, the washed (as above) antibody-antigen-enzyme complex is preferably exposed to the colorigenic substrate, such as AttoPhos from Roche for about 10 minutes at room temperature, the reaction may be stopped with a low pH buffer such as 50 mM Tris-HCl pH 5.5, or can instead be directly assayed. The amount of specifically bound OBcl5.pr polypeptide or DAP3.pr polypeptide is for example determined by measurement of the amount of the enzymatic reaction product in each well following excitation at the appropriate wavelength in the spectrophotometer (420 nm in this case). Measurement is preferably made in the plate reader at the emission wavelength (560 nm in this case). Preferably included in the diagnostic is an OBcl5 protein standard or a DAP3 protein standard, such as purified recombinant OBcl5.pr polypeptide or DAP3.pr polypeptide, for example. A dilution series of this protein standard is preferably included in parallel in the ELISA as a control for the reactions and to deduce a protein standard curve for comparison of polypeptide expression levels as is well known in the art. A concentration range corresponding indicative of the particular liver disorder(s) should preferably be provided in the diagnostic. In addition, a comparable fluid or tissue extract should preferably also be included as a control for such ELISA test. This may preferably include serum or plasma from non-diseased individuals and/or serum, plasma or tissue extracts from an appropriate animal model. Such ELISA detection diagnostics are common in the art (see for example, Hauschild et al., 2001, Cancer Res. 158:169-77). The sample:control protein levels determined by ELISA are compared with ELISA-determined disorder specific protein expression ratio values preferably determined in pathologist-confirmed tissues of patients suffering from a disorder according to the invention in relation to control samples. In case the protein level of the sample:control essentially matches the disorder specific protein expression ratio values such matching is preferably indicative of the patient suffering from the disorder. Preferably such diagnosis is carried out for more than 1 polypeptide according to the invention.

In addition the diagnostic may be directed to detecting an endogenous antibody directed against a polypeptide according to the invention, or a functional variant thereof or fragment thereof present in the sample isolated from a patient which antibody or fragment thereof is directed against a polypeptide according to the invention. Detection of such autoimmune antibodies may be accomplished by methods generally known to the skilled artisan, e.g. by immunoaffinity assays such the ELISA described in detail above using polypeptides according to the invention or functional variants thereof or parts thereof as a probe. The presence of such autoimmune antibodies is indicative of the patient suffering from a disorder according to the invention.

In addition or alternatively, a relevant diagnostic kit based upon immunohistochemical detection of at least one polypeptide according to the invention can be formulated. In such a kit, for example a purified antibody or antibodies specific for the polypeptide(s) according to the invention can be included as well as preferably the reagents necessary to detect the binding of the antibody(ies) to patient cells or tissue sections. These reagents include, for example a specific anti-species and subtype specific secondary antibody—directed against a polypeptide according to the invention of a functional variant thereof—preferably coupled to an enzyme capable of catalysis of e.g. a colorigenic substrate or coupled to a fluorophore (such as Texas Red, for example). Perferably the enzymatic substrate would also be included as well as washing and incubation buffers. An additional optional component of such a kit may be a section of positive control tissue, e.g. liver, or tissues or a section from a packed pellet of cells specifically expressing the polypeptide(s) as a positive tissue control. Instructions provided would include preferred and/or alternative methods of antigen retrieval for detection of the polypeptide(s) according to the invention or e.g., indication that frozen, rather than formalin fixed and paraffin-embedded tissue material should be employed. In this case, recommendations would preferably be included for fixation of frozen tissue sample sections, such as immersion in ice-cold acetone for 10 minutes. Further instructions would preferably provide recommendations for the concentration of antibodies to use in the detection of the gene product(s) as well as e.g., recommended and suggested incubation times and temperatures for exposure of the tissue to the immunological reagents provided. Preferred reaction buffers for the antibody incubations, such as 0.01%-0.1% tween-20 comprising phosphate buffered saline including 3% normal sheep serum, could also be included. Further, specific conditions for washing of the tissue sections prior to and following incubation in the specific antibody would be preferably included, such as for example, 4 washes with 0.1% tween-20 comprising phosphate buffered saline for 5 minutes each. Such immunohistochemical detection protocols are known to a person skilled in the art. In general the kit would preferably include a panel of images of specific immunohistochemical staining results from positive and negative tissue examples and in particular tables indicating which result is indicative of the patient suffering from the disorder to be diagnosed as a user guide. Utilization of such a kit would preferably rule out, support or confirm diagnoses of the aforementioned liver disorders, liver cancer, or epithelial cancers according to the invention.

As specified above for nucleic acid-based diagnostic approaches, diagnostics based on detection and/or quantitation of polypeptides according to the invention may include 1 or more of such polypeptides. Moreover, simultaneous detection of such polypeptides together with other peptides implicated in the disorders according to the invention may be employed in such diagnostics.

It will be apparent to those skilled in the art that various modifications can be made to the compositions and processes of this invention. Thus, it is intended that the present invention cover such modifications and variations, provided they come within the scope of the appended claims and their equivalents. All publications cited herein are incorporated in their entireties by reference.

1. An isolated polypeptide comprising a sequence according to SEQ ID 2, or a functional variant thereof.

2. A fusion protein comprising a polypeptide according to claim 1.

3. An isolated nucleic acid, or a variant thereof encoding the polypeptide according to claim 1.

4. The nucleic acid according to claim 3, wherein the nucleic acid is a single-stranded or double-stranded RNA.

5. The nucleic acid according to claim 3, wherein the nucleic acid comprises a nucleic acid according to SEQ ID 11.

6. A vector, comprising a nucleic acid selected from the group consisting of the nucleic acid according to claim 3, and a nucleic acid coding for a polypeptide according to the SEQ ID 1 to 9 or SEQ ID 47.

7. The vector according to claim 6, wherein the vector is selected from the group consisting of a knock-out gene construct, a plasmid, a shuttle vector, a phagemid, a cosmid, a viral vector, and an expression vector.

8. A cell comprising the nucleic acid according to claim 3.

9. A cell comprising the vector according to claim 6.

10. The cell according to claim 9, wherein the cell is a transgenic embryonic non-human stem cell.

11. A transgenic non-human mammal comprising the nucleic acid according to claim 3.

12. An antibody or an antibody fragment thereof, wherein the antibody is directed against the polypeptide according to claim 1 or against the nucleic acid according to claim 3.

13. A nucleic acid which comprises a nucleic acid having a sequence complementary to the nucleic acid according to claim 3 or a non-functional mutant variant of the nucleic acid according to claim 3.

14. The nucleic acid according to claim 13, wherein the nucleic acid having a complementary sequence is an antisense molecule or an RNA interference molecule.

15. A vector comprising the nucleic acid according to claim 13.

16. The vector according to claim 15, wherein the vector is selected from the group consisting of a plasmid, a shuttle vector, a phagemid, a cosmid, a viral vector, and an expression vector.

17. A cell comprising the nucleic acid according to claim 13.

18. A cell comprising the vector according to claim 15.

19. A diagnostic comprising at least one compound selected from the group consisting of the polypeptide according to claim 1, a polypeptide according to SEQ ID 1 to 9 or SEQ ID 47, a nucleic acid encoding one of the aforementioned polypeptides, a variant of one of the aforementioned nucleic acids, and an antibody or an antibody fragment directed against one of the aforementioned polypeptides, combined or together with suitable additives or auxiliaries.

20. The diagnostic according to claim 19, wherein the nucleic acid is a probe.

21. The diagnostic according to claim 20, wherein the probe is a DNA probe.

22. A pharmaceutical composition comprising at least one component selected from the group consisting of the polypeptide according to claim 1, a polypeptide according to SEQ ID 1 to 9 or SEQ ID 47, a functional variant of one of the aforementioned polypeptides, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of the antibody directed against one of the aforementioned polypeptides, an antibody or a fragment of the antibody directed against a functional variant of one of the aforementioned polypeptides, a vector comprising a nucleic acid coding for one of the aforementioned antibodies, a cell comprising the vector comprising a nucleic acid coding for one of the aforementioned antibodies, and a cell comprising the vector comprising a nucleic acid coding for one of the aforementioned antibody fragments, combined or together with suitable additives or auxiliaries.

23. The pharmaceutical composition according to claim 22, wherein the nucleic acid having a complementary sequence is an antisense molecule or an RNA interference molecule.

24. A method of diagnosis of a liver disorder or an epithelial cancer, wherein at least one compound selected from the group consisting of a polypeptide according to the sequence of SEQ ID 1 to SEQ ID 9 or SEQ ID 47, a functional variant of one of the aforementioned polypeptides, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, an antibody or a fragment of the antibody directed against one of the aforementioned polypeptides, and an antibody or a fragment of the antibody directed against a functional variant of one of the aforementioned polypeptides, is identified in the sample of a patient and compared with at least one compound of a reference library or of a reference sample.

25. The method according to claim 24, wherein the liver disorder, is a disorder selected from the group consisting of cirrhosis, alcoholic liver disease, chronic hepatitis, Wilson's Disease, heamochromatosis, hepatocellular carcinoma, benign liver neoplasms, and focal nodular hyperplasia.

26. The method according to claim 24, wherein the epithelial cancer is an adenocarcinoma of an organ selected from the group consisting of the lung, the stomach, the kidney, the colon, the prostate, the skin, and the breast.

27. A method of treating a patient suffering from a liver disorder or an epithelial cancer, wherein at least one component selected from the group consisting of a polypeptide according SEQ ID 1 to 9 or SEQ ID 47, a functional variant of one of the aforementioned polypeptides, a nucleic acid encoding one of the aforementioned polypeptides, a variant of one of the aforementioned nucleic acids, a nucleic acid which is a non-functional mutant variant of one of the aforementioned nucleic acids, a nucleic acid having a sequence complementary to one of the aforementioned nucleic acids, a vector comprising one of the aforementioned nucleic acids, a cell comprising one of the aforementioned nucleic acids, a cell comprising the aforementioned vector, an antibody or a fragment of the antibody directed against one of the aforementioned polypeptides, an antibody or a fragment of the antibody directed against a functional variant of one of the aforementioned polypeptides, a vector comprising a nucleic acid coding for the antibody, a cell comprising the vector comprising a nucleic acid coding for the antibody, and a cell comprising the vector comprising a nucleic acid coding for the antibody fragment, combined or together with suitable additives or auxiliaries, is administered to the patient in need of a the treatment in a therapeutically effective amount.

28. The method of treating according to claim 27, wherein the nucleic acid having a complementary sequence is an antisense molecule or an RNA interference molecule.

29. The method of treating according to claim 28, wherein the RNA interference molecule is administered in the form of a double stranded RNA or a vector expressing the double stranded RNA.

30. The method according to claim 29, wherein the RNA interference molecule has a size range selected from the group consisting of from 15 to 30 nucleotides.

31. The method according to claim 27, wherein the liver disorder, is a disorder selected from the group consisting of cirrhosis, alcoholic liver disease, chronic hepatitis, Wilson's Disease, heamochromatosis, hepatocellular carcinoma, benign liver neoplasms, and focal nodular hyperplasia.

32. The method according to claim 27, wherein the epithelial cancer is an adenocarcinoma of an organ selected from the group consisting of the lung, the stomach, the kidney, the colon, the prostate, the skin, and the breast.

33. A method of stimulating an immune response in a patient suffering from a liver disorder or an epithelial cancer, to a polypeptide according to the sequence of SEQ ID 1 to SEQ ID 9 or SEQ ID 47, or a functional variant thereof, wherein at least one component selected from the group consisting of a polypeptide according to the sequence of SEQ ID 1 to SEQ ID 9 or SEQ ID. No. 47, a functional variant thereof, a nucleic acid encoding one of the aforementioned polypeptides, a variant of one of the aforementioned nucleic acids, a vector comprising one of the aforementioned nucleic acids, a cell comprising one of the aforementioned nucleic acids, and a cell comprising the aforementioned vector, is administered to the patient in need of such treatment in an amount effective to stimulate the immune response in the patient.

34. A method for identifying at least one nucleic acid according to SEQ ID 10 to SEQ ID 19, or a variant thereof differentially expressed in a sample isolated from a patient relative to a reference library or a reference sample comprising the following steps:
(a) detecting the expression of at least one nucleic acid according to SEQ ID 10 to SEQ ID 19, or a variant thereof in a sample isolated from a patient,
(b) comparing the expression of said nucleic acid(s) detected in step (a) with the expression of the said nucleic acid(s) in a reference library or in a reference sample,
(c) identifying said nucleic acid(s) which is (are) differentially expressed in the sample isolated from the patient compared to the reference library or the reference sample.

35. A method of diagnosing a liver disorder or an epithelial cancer comprising the following steps:
(a) detecting the expression of at least one nucleic acid according to SEQ ID 10 to SEQ ID 19, or a variant thereof in a sample isolated from a patient,
(b) comparing the expression of said nucleic acid(s) detected in step (a) with the expression of said nucleic acid(s) in a reference library or in a reference sample,
(c) identifying said(s) nucleic acid which is (are) differentially expressed in the sample isolated from the patient compared to the reference library or the reference sample, and
(d) matching said nucleic acid(s) identified in step (c) said nucleic acid(s) differentially expressed in a pathologic reference sample or pathologic reference library,
wherein the matched nucleic acid(s) is (are) indicative of the patient suffering from a liver disorder or an epithelial cancer.

36. The method according to claim 35, wherein step (a) at least 2 nucleic acids are identified.

37. The method according to claim 35, wherein in step (a) the detection of said nucleic acid(s) is (are) by PCR based detection or by a hybridization assay.

38. The method according to claim 35, wherein in step (b) the expression of said nucleic acid(s) is compared by a method selected from the group consisting of solid-phase based screening methods, hybridization, subtractive hybridization, differential display, and RNase protection assay.

39. The method according to claim 35, wherein the sample isolated from the patient is selected from the group consisting of liver tissue, a liver cell, tissue from another organ subject to cancerous transformation, a cell from this organ, blood, serum, plasma, ascitic fluid, pleural effusion, cerebral spinal fluid, saliva, urine, semen, and feces.

40. The method according to claim 35, wherein the reference sample is isolated from a source selected from a non-diseased sample of the same patient and a non-diseased sample from another subject.

41. The method according to claim 35, wherein the reference sample is selected from the group consisting of liver tissue, a liver cell, blood, serum, plasma, ascitic fluid, pleural effusion, cerebral spinal fluid, saliva, urine, semen, and feces.

42. The method according to claim 35, wherein the reference library is an expression library or a data base comprising clones or data on liver disorder-specific expression of said nucleic acid(s) of step (a).

43. The method according to claim 35, wherein the pathologic reference sample is isolated from a source selected from a diseased sample from another patient suffering from a liver disorder or epithelial cancer.

44. The method according to claim 35, wherein the pathologic reference library is a data base comprising data on differential expression of said nucleic acid(s) in step (a) in samples isolated from another patient suffering from a liver disorder or epithelial cancer relative to control expression in a reference sample or reference library.

45. The method according to claim 35, wherein the liver disorder, is a disorder selected from the group consisting of hepatocellular carcinoma, benign liver neoplasms, and cirrhosis.

46. The method according to claim 35, wherein the epithelial cancer is an adenocarcinoma of an organ selected from the group consisting of the lung, the stomach, the kidney, the colon, the prostate, the skin and the breast.

47. A method for identifying at least one polypeptide according to SEQ ID 1 to SEQ ID 9 or SEQ ID 47, or a functional variant thereof differentially expressed in a sample isolated from a patient relative to a reference library or a reference sample comprising the following steps:
(a). detecting the expression of at least one polypeptide according to SEQ ID 1 to SEQ ID 9 or SEQ ID 47, or a functional variant thereof in a sample isolated from a patient,
(b) comparing the expression of said polypeptide(s) detected in step (a) with the expression of said polypeptide(s) in a reference library or in a reference sample,
(c) identifying said polypeptide(s) which is (are) differentially expressed in the sample isolated from the patient compared to the reference library or the reference sample.

48. A method of diagnosing a liver disorder or epithelial cancers comprising the following steps:
(a) detecting the expression of at least one polypeptide according to SEQ ID 1 to SEQ ID 9 or SEQ ID No. 47, or functional variants thereof in a sample isolated from a patient,
(b) comparing the expression of said polypeptide(s) detected in step (a) with the expression of said polypeptide(s) in a reference library or in a reference sample,
(c) identifying said polypeptide(s) which is (are) differentially expressed in the sample isolated from the patient compared to the reference library or the reference sample, and
(d) matching said polypeptide i(s)dentified in step (c) with said polypeptide(s) differentially expressed in a pathologic reference sample or pathologic reference library,
wherein the matched polypeptide(s) are indicative of the patient suffering from a liver disorder, or an epithelial cancer.

49. The method according to claim 48, wherein at least 2 polypeptides are identified.

50. The method according to claim 48, wherein the polypeptides are detected by a method selected from the group consisting of gel electrophoresis, chromatographic techniques, immunoblot analysis, immunohistochemistry, enzyme based immunoassay, surface plasmon resonance, HPLC, mass spectroscopy, immunohistochemistry, and enzyme based immunoassay.

51. The method according to claim 48, wherein the polypeptides are compared by a method selected from the group consisting of two dimensional gel electrophoresis, chromatographic separation techniques, immunoblot analysis, surface plasmon resonance, immunohistochemistry, and enzyme based immunoassay.

52. The method according to claim 48, wherein the sample isolated from a patient is selected from the group consisting of liver tissue, a liver cell, tissue from another organ subject to cancerous transformation, a cell from this organ, blood, serum, plasma, ascitic fluid, pleural effusion, cerebral spinal fluid, saliva, urine, semen, and feces.

53. The method according to claim 48, wherein the reference sample is isolated is from a source selected from a non-diseased sample of the same patient and a non-diseased sample from another subject.

54. The method according to claim 48, wherein the reference sample is selected from the group consisting of liver tissue, a liver cell, blood, serum, plasma, ascitic fluid, pleural effusion, cerebral spinal fluid, saliva, urine, semen, and feces.

55. The method according to claim 48, wherein the reference library is an expression library or a data base comprising clones or data on liver disorder-specific expression of said polypeptide(s) of step (a).

56. The method according to claim 48, wherein the pathologic reference sample is isolated from a source selected from a diseased sample from another patient suffering from a liver disorder and epithelial cancer.

57. The method according to claim 48, wherein the pathologic reference library is a data base comprising data on differential expression of said polypeptide(s) of step (a) in samples isolated from another patient, suffering from a liver disorder or epithelial cancer relative to control expression in a reference sample or reference library.

58. The method according to claim 48, wherein the liver disorders is a disorder selected from the group consisting of hepatocellular carcinoma, benign liver neoplasms, and cirrhosis.

59. The method according to claim 48, wherein the epithelial cancer is an adenocarcinoma of an organ selected from the group consisting of the lung, the stomach, the kidney, the colon, the prostate, the skin, and the breast.

60. A method of preventing a patient from developing a liver disorder or an epithelial cancer, wherein at least one component selected from the group consisting of a polypeptide according to the sequence of SEQ ID 1 to SEQ ID 9 or SEQ ID 47, a functional variant thereof, a nucleic acid encoding one of the aforementioned polypeptides, a variant of one of the aforementioned nucleic acids, a nucleic acid having a sequence complementary to one of the aforementioned nucleic acids, a nucleic acid which is a non-functional mutant variant of one of the aforementioned nucleic acids, a vector comprising one of the aforementioned nucleic acids, or a variant thereof, a cell comprising one of the aforementioned nucleic acids, or a variant thereof, and a cell comprising the aforementioned vector, is administered to the patient in need of such preventive treatment in a therapeutically effective amount.

61. A method of identifying a pharmacologically active compound comprising the following steps:
(a) providing at least one polypeptide according to the SEQ ID 1 to 9 or 47, or a functional variant thereof,
(b) contacting said polypeptide(s) with suspected to be pharmacologically active compound(s),
(c) assaying the interaction of said polypeptide(s) of step (a) with said compound(s) suspected to be pharmacologically active,
(d) identifying said compound(s) suspected to be pharmacologically active which directly or indirectly interact with said polypeptide(s) of step (a).

62. The method according to claim 61, wherein said polypeptide(s) of step (a) is (are) attached to a column, said polypeptide(s) is (are) attached to an array, contained in an electrophoresis gel, attached to a membrane, or is (are) expressed by a cell.

63. The method according to claim 61, wherein the interaction is assayed enzyme or fluorescence based cellular reporter methods.

64. The method according to claim 61, wherein the interaction is assayed by surface plasmon resonance, HPL, or mass spectroscopy.

65. The method according to claim 61, wherein the direct or indirect functional interaction of step (d) is selected from the group consisting of induction of the expression of said polypeptide(s) of step (a), inhibition of said polypeptide(s), activation of the function of said polypeptide(s), and inhibition of the function of said polypeptide(s).
